(19) United States
(12) Patent Application Publication
(10) Pub. No.: US 2022/0267448 A1
(43) Pub. Date: Aug. 25, 2022

(54) CELL CULTURE METHODS AND COMPOSITIONS FOR ANTIBODY PRODUCTION

(71) Applicant: Takeda Pharmaceutical Company Limited, Osaka (JP)

(72) Inventors: Yogender Kumar Gowtham, Cary, NC (US); Sokha Susan Lay, Roslindale, MA (US); Edmund G. Scarfo, Bradford, MA (US); Nandita Vishwanathan, Vevey (CH)

(21) Appl. No.: 17/596,418

(22) PCT Filed: Jun. 10, 2020
(86) PCT No.: PCT/US2020/037080

§ 371 Date: Dec. 9, 2021

Related U.S. Application Data
(60) Provisional application No. 62/859,563, filed on Jun. 10, 2019.
(60) Provisional application No. 62/859,596, filed on Jun. 10, 2019.

(51) Int. Cl. C07K 16/28; A61K 9/19; A61P 35/00

(52) U.S. Cl. CPC C07K 16/2839; A61P 35/00; A61K 9/19

(57) ABSTRACT

The invention provides cell culture methods for producing an anti-α4β7 antibody, e.g., vedolizumab, as well as compositions thereof.

### RELATED APPLICATIONS

This application is a U.S. National Phase Application under 35 U.S.C. § 371 of International Application No. PCT/US2020/037080, filed on Jun. 10, 2020, which claims priority to U.S. Provisional Application No. 62/859,563, filed on Jun. 10, 2019, and to U.S. Provisional Application No. 62/859,596, filed on Jun. 10, 2019. The entire contents of the foregoing applications are incorporated herein by reference.

### SEQUENCE LISTING

The instant application contains a Sequence Listing which has been submitted in ASCII format via EFS-Web and is hereby incorporated by reference in its entirety. Said ASCII copy, created on Jun. 10, 2020, is named “T103022_1110US_SL.TXT” and is 14.0 kilobytes in size. The entire contents of the Sequence Listing in the sequence listing.txt file are incorporated herein.

### FIELD OF THE INVENTION

The present invention relates to methods and compositions for producing an anti-α4β7 antibody in mammalian host cells.

### BACKGROUND

Mammalian cell culture technology is commonly used in the production of therapeutic biologics, including therapeutic monoclonal antibodies. Mammalian cells are usually preferred in the pharmaceutical industry over other forms of eukaryotic cells (such as yeast) or prokaryotic cells (such as bacteria) for protein production because proteins produced in mammalian cells generally have post-translational modifications that are more similar to proteins produced in humans. Mammalian cell culture can, however, be difficult as these cells present a number of challenges, particularly in the context of therapeutic antibodies that are manufactured on a commercial scale for use in humans. Production methods must maximize antibody yield from the cells, while maintaining safety of the protein product, as well as efficiency and cost-effectiveness. Thus, production demands are important, as is the need to maintain the desired quality attributes of the product such as the glycosylation profile, aggregate levels, charge heterogeneity, and amino acid sequence integrity (Li et al., 2010, mAbs, 2(5):466-477).

Identifying cell culture parameters that can address challenges associated with producing a therapeutic antibody, including maintaining a high quality drug product while producing enough of the protein product to meet manufacturing demands and therapeutic requirements, can be difficult given the complexity of the cell culturing process.

### SUMMARY OF THE INVENTION

Although mammalian cell culture processes have been the subject of study over the past several decades, there remains a need for improvements in the large-scale commercial production of recombinant antibodies. Increases in cell viability, longevity and specific productivity of mammalian host cell cultures, and improvements in the titer of the recombinant proteins produced have a genuine impact on the price of the recombinant protein produced, and, in the case of therapeutic proteins, the price and availability of drug products. Further, such increases can be especially challenging given the need to maintain consistency in the quality of the therapeutic antibody being produced.

The invention provided herein discloses, inter alia, cell culture methods and compositions for producing an anti-α4β7 antibody, such as vedolizumab, in mammalian host cells. Also provided herein are compositions comprising an anti-α4β7 antibody, such as vedolizumab, obtained using said methods.

In one aspect, the invention provides a method of producing a composition comprising a humanized anti-α4β7 antibody, said method comprising culturing a mammalian host cell in a production medium, and adding a supplement comprising uridine, manganese, and galactose to the production medium, thereby producing a composition comprising the humanized anti-α4β7 antibody, wherein the mammalian host cell is genetically engineered to express a humanized anti-α4β7 antibody which is an IgG1 antibody, comprises a heavy chain variable region comprising a CDR3 domain as set forth in SEQ ID NO: 4, a CDR2 domain as set forth in SEQ ID NO: 3, and a CDR1 domain as set forth in SEQ ID NO: 2; and comprises a light chain variable region comprising a CDR3 domain as set forth in SEQ ID NO: 8, a CDR2 domain as set forth in SEQ ID NO: 7, and a CDR1 domain as set forth in SEQ ID NO: 6.

In some embodiments of the aforementioned aspect, the method is a method of producing a composition having a decreased amount of basic isoform (as determined by Cation Exchange Chromatography (CEX)) of the humanized anti-α4β7 antibody, said method comprising said culturing of a mammalian host cell in a production medium, and said adding of a supplement comprising uridine, manganese, and galactose to the production medium, thereby producing a composition having a decreased amount of basic isoform of the humanized anti-α4β7 antibody in comparison to a control mammalian host cell expressing the humanized anti-α4β7 antibody that is cultured in the absence of the supplement.

In some embodiments of the above aspect, the invention features a method of producing a composition having about 16% or less basic isoform (as determined by CEX) of the humanized anti-α4β7 antibody, said method comprising said culturing of a mammalian host cell in a production medium, and said adding of a supplement comprising uridine, manganese, and galactose to the production medium, thereby producing a composition having about 16% or less basic isoform of the humanized anti-α4β7 antibody.

In one embodiment, the composition comprises about 14% or less basic isoform of the humanized anti-α4β7 antibody.

In another embodiment, the composition comprises about 13% or less basic isoform of the humanized anti-α4β7 antibody.

In one embodiment, the supplement is added to the production medium or is added to a feed medium which is subsequently added to the production medium.

In one embodiment, the cumulative concentration of uridine added in the production medium between supplementation and harvest is about 1 to about 7 mM; wherein the cumulative concentration of manganese added in the production medium between supplementation and harvest is about 0.002 to about 0.015 mM; and/or wherein the cumulative concentration of galactose added in the production medium between supplementation and harvest is about 3 to about 20 mM. In certain embodiments, the feed medium further comprises zinc. In one embodiment, the cumulative concentration of zinc added in the production medium between supplementation and harvest is about 0.05 mM to about 0.045 mM.

In one embodiment, manganese is added as a supplement multiple times to the production medium by about 0.1 to 10 μM, about 0.2 to 1.5 μM, about 0.2 to 5 μM, about 0.25 to 2 μM, about 0.3 to 1.2 μM, or about 0.3 to 0.8 μM with each addition. IN certain embodiments, manganese is added as a supplement multiple times to the production medium by about 0.2 to 1.5 μM with each addition.

In one embodiment, uridine is added as a supplement multiple times to the production medium by about 25 to 1000 μM, about 75 to 750 μM, about 55 to 620 μM, about 100 to 600 μM, about 150 to 450 μM, about 100 to 700 μM, about 100 to 600 μM, or about 170 to 630 μM with each addition. In certain embodiments, uridine is added as a supplement multiple times to the production medium by about 100 to 700 μM.

In one embodiment, galactose is added as a supplement multiple times to the production medium by about 0.1 to 10 mM, 0.2 to 7.5 mM, 0.5 to 5 mM, 0.4 to 2.8 mM, 0.5 to 3.5 mM, 0.7 to 2.9 mM, 0.75 to 2.5 mM or by about 1.2 mM or 1.4 mM with each addition. In certain embodiments, galactose is added as a supplement multiple times to the production medium by about 0.5 to 3.5 mM.

In one embodiment, the supplement is added daily or every two days. In certain embodiments, the supplement is added beginning on day 4 of the production phase culture.

In one embodiment, uridine is added to the feed medium at a final concentration of about 15 to 120 mM. In one embodiment, the uridine is added to the feed medium to a final concentration of about 20 to 70 mM uridine. In one embodiment, the uridine is added to the feed medium to a final concentration of about 1 to 40 mM uridine.

In one embodiment, manganese is added to the feed medium at a final concentration of about 0.02 to 0.3 mM. In one embodiment, manganese is added to the feed medium to a final concentration of about 0.04 to 0.15 mM. In one embodiment, manganese is added to the feed medium to a final concentration of about 0.0001 to 0.1 mM.

In a further embodiment embodiment, galactose is added to the feed medium to a final concentration of about 85 mM to 600 mM. In one embodiment, galactose is added to the feed medium to a final concentration of about 160 to 340 mM. In one embodiment, galactose is added to the feed medium to a final concentration of about 50 to 150 mM.

In another embodiment, the feed medium further comprises zinc. In one embodiment, the concentration of zinc in the feed medium is about 90 μM to 120 μM. In one embodiment, the concentration of zinc in the feed medium is about 50 μM to 150 μM.

In one embodiment, the method further decreases the percentage of acidic species of the humanized anti-α4β7 antibody relative to the percentage of acidic species produced in a control mammalian host cell expressing the humanized anti-α4β7 antibody that is cultured in the absence of the supplement

In one embodiment, the method further increases the percentage of main isoform species of the humanized anti-α4β7 antibody relative to the percentage of main isoform species produced in the absence of a feed medium comprising uridine, manganese, and galactose added to the production medium.

In one embodiment, the method is a fed batch method.

In one embodiment, the feed medium is added to the production medium beginning on about day four of the production phase.

In another aspect, the invention provides a method of producing a composition comprising a humanized anti-α4β7 antibody, said method comprising culturing a mammalian host cell in a production medium comprising zinc, thereby producing a composition comprising the humanized anti-α4β7 antibody, wherein the mammalian host cell is genetically engineered to express a humanized anti-α4β7 antibody which is an IgG1 antibody; comprises a heavy chain variable region comprising a CDR3 domain as set forth in SEQ ID NO: 4, a CDR2 domain as set forth in SEQ ID NO: 3, and a CDR1 domain as set forth in SEQ ID NO: 2; and comprises a light chain variable region comprising a CDR3 domain as set forth in SEQ ID NO: 8, a CDR2 domain as set forth in SEQ ID NO: 7, and a CDR1 domain as set forth in SEQ ID NO: 6.

In some embodiments of the aforementioned aspect, the method is a method of producing a composition having a decreased amount of basic isoform (as determined by Cation Exchange Chromatography (CEX)) of the humanized anti-α4β7 antibody, said method comprising said culturing of a mammalian host cell in a production medium comprising zinc, thereby producing a composition having a decreased amount of basic isoform of the humanized anti-α4β7 antibody in comparison to a control mammalian host cell expressing the humanized anti-α4β7 antibody that is cultured in the absence of zinc.

In some embodiments of the above aspect, the method is a method of producing a composition having about 16% or less basic isoform (as determined by CEX) of the humanized anti-α4β7 antibody, said method comprising said culturing of a mammalian host cell in a production medium comprising zinc, thereby producing a composition having about 16% or less basic isoform of the humanized anti-α4β7 antibody.

In one embodiment, the composition comprises about 14% or less basic isoform of the humanized anti-α4β7 antibody.

In one embodiment, the composition comprises about 13% or less basic isoform of the humanized anti-α4β7 antibody.

In one embodiment, the concentration of zinc in the production medium is 2 μM to 60 μM.

In a further embodiment, the method comprises supplementing the production medium with zinc by adding a feed medium comprising zinc to the production medium. In one embodiment, the feed medium is added to the production medium beginning on about day four of the production phase.

In one embodiment, the concentration of zinc in the feed medium is about 90 μM to 120 μM.

In one embodiment, the production medium comprises 5.0 to 8.8 g/L lysine and 3.0 to 12.0 g/L arginine. In one embodiment, the production medium comprises 4.5 to 5.5 g/L lysine. In one embodiment, the production medium comprises 5.5 to 8.8 g/L lysine. In one embodiment, the production medium comprises 5.4 to 7.4 g/L arginine. In one embodiment, the production medium comprises 7.4 to 12 g/L arginine.

In a further aspect, the invention features a method of producing a composition comprising a humanized anti-α4β7 antibody, said method comprising culturing a mammalian host cell in a production medium in a production phase, such that a composition comprising the humanized anti-α4β7 antibody is produced, wherein the production medium has an average temperature of about 37 degrees Celsius, wherein the host cell is genetically engineered to express a humanized IgG1 anti-α4β7 antibody, wherein the humanized anti-α4β7 comprises a heavy chain variable region comprising a CDR3 domain as set forth in SEQ ID NO: 4, a CDR2 domain as set forth in SEQ ID NO: 3, and a CDR1 domain as set forth in SEQ ID NO: 2; and comprises a light chain variable region comprising a CDR3 domain as set forth in SEQ ID NO: 8, a CDR2 domain as set forth in SEQ ID NO: 7, and a CDR1 domain as set forth in SEQ ID NO: 6.

In some embodiments of the aforementioned aspect, the method is a method of producing a composition comprising 2.5% or less HMW species (as determined by SEC) of the humanized anti-α4β7 antibody, said method comprising said culturing of a mammalian host cell in a production medium in a production phase, such that a composition comprising 2.5% or less HMW species (as determined by SEC) of the humanized anti-α4β7 antibody is produced.

In still another aspect, the invention provides a method of producing a composition comprising a humanized anti-α4β7 antibody, said method comprising culturing a mammalian host cell in a growth medium in an expansion phase, wherein the mammalian host cell is genetically engineered to express a humanized anti-α4β7 antibody, and culturing the mammalian host cell in a production medium in a production phase, such that a composition comprising the humanized anti-α4β7 antibody is produced, wherein the mammalian host cell is cultured at a temperature that is approximately the same in both the expansion phase and the production phase, and wherein the humanized anti-α4β7 antibody is an IgG1 antibody; comprises a heavy chain variable region comprising a CDR3 domain as set forth in SEQ ID NO: 4, a CDR2 domain as set forth in SEQ ID NO: 3, and a CDR1 domain as set forth in SEQ ID NO: 2; and comprises a light chain variable region comprising a CDR3 domain as set forth in SEQ ID NO: 8, a CDR2 domain as set forth in SEQ ID NO: 7, and a CDR1 domain as set forth in SEQ ID NO: 6.

In some embodiments of the aforementioned aspect, the method is a method of producing a composition comprising high levels of monomer (as determined by SEC) of the humanized anti-α4β7 antibody, said method comprising said culturing of a mammalian host cell in a growth medium in an expansion phase, wherein the mammalian host cell is genetically engineered to express a humanized anti-α4β7 antibody, and said culturing of the mammalian host cell in a production medium in a production phase, such that a composition comprising high levels of monomer (as determined by SEC) of the humanized anti-α4β7 antibody is produced.

In one embodiment, the temperature is from 36 to 38 degrees Celsius. In another embodiment, the average temperature is from 36.5 to 37.5 degrees Celsius. In still another embodiment, the temperature is an average temperature of about 37 degrees Celsius.

In one embodiment, the production medium of the methods disclosed herein has a temperature ranging from 36 to 38 degrees Celsius. In one embodiment, the temperature ranges from 36.5 to 37.5 degrees Celsius. In one embodiment, the temperature is an average temperature of about 37 degrees Celsius. In one embodiment, the production medium has a pH ranging from 6.5 to 7.

In one embodiment, the production medium of the methods disclosed herein a pH ranging from 6.8 to 7.0.

In one embodiment, the production medium of the methods disclosed herein has a glucose level that is maintained at about 7 g/L or less a during the production phase.

In one embodiment, the production phase is 14 days or less. In another embodiment, the production phase ranges from 10 days to 17 days.

In some embodiments of the above aspects, the method is performed in a large scale bioreactor. In certain embodiments, the large scale bioreactor is selected from the group consisting of a 200 liter (L) bioreactor, a 2000 L bioreactor, a 3000L, and a 6000 L bioreactor.

In some embodiments, the production phase results in a titer of the humanized anti-a4(37 antibody of greater than 3 g/L. In certain embodiments, the titer of the humanized anti-α4β7 antibody is about 3 to about 8 g/L. In other embodiments, the titer of the humanized anti-α4β7 antibody is about 5 to about 7 g/L.

In some embodiments of the above aspects, the mammalian host cell is a Chinese Hamster Ovary (CHO) cell. In certain embodiments, the CHO cell is a GS-CHO cell.

In some embodiments of the above aspects, the humanized anti-α4β7 antibody comprises a heavy chain variable domain comprising an amino acid sequence as set forth in SEQ ID NO: 1, and comprises a light chain variable domain comprising an amino acid sequence as set forth in SEQ ID NO: 5.

In some embodiments of the above aspects, the humanized anti-α4β7 antibody is vedolizumab.

In some embodiments of the aforementioned aspects, the method comprises harvesting and purification of the antibody. In some such embodiments, the purification comprises (i) purification steps that remove any cellular debris, unwanted proteins, salts, minerals or other undesirable elements, and (ii) purification of the antibody from contaminant soluble proteins and polypeptides. In certain embodiments, the method further comprises preparing a pharmaceutical formulation of the purified antibody which is suitable for human therapeutic use.

In particular embodiments, the pharmaceutical formulation is a liquid pharmaceutical formulation. In some such embodiments, the liquid pharmaceutical formulation is prepared by ultrafiltration/diafiltration.

In other embodiments, the pharmaceutical formulation is a lyophilized dry antibody formulation. In some such embodiments, the pharmaceutical formulation of the antibody is a dry antibody formulation lyophilized from a liquid pharmaceutical antibody formulation prepared by ultrafiltration/diafiltration following the purification.

In some embodiments, the invention provides a method of producing a composition having a decreased amount of a G0F glycoform (as determined by Hydrophilic Interaction Chromatography (HILIC)) of the humanized anti-α4β7 antibody, said method comprising said culturing of a mammalian host cell in a production medium, and said adding of a supplement comprising uridine, manganese, and galactose to the production medium, thereby producing a composition having a decreased amount of the G0F glycoform of the humanized anti-α4β7 antibody in comparison to a control mammalian host cell expressing the humanized anti-α4β7 antibody that is cultured in the absence of the supplement.

In one embodiment, the composition comprises at least about a 15% decreased level of the G0F glycoform of the humanized anti-α4β7 antibody in comparison to a control mammalian host cell expressing the humanized anti-α4β7 antibody that is cultured in the absence of the supplement.

In one embodiment, the composition comprises at least about a 20% decrease in the G0F glycoform of the humanized anti-α4β7 antibody in comparison to a control mammalian host cell expressing the humanized anti-α4β7 antibody that is cultured in the absence of the supplement.

In some embodiments, provided herein is a method of producing a composition having about 65% or less G0F glycoform (as determined by HILIC) of the humanized anti-α4β7 antibody, said method comprising said culturing of a mammalian host cell in a production medium, and said adding of a supplement comprising uridine, manganese, and galactose to the production medium, thereby producing a composition having about 65% or less G0F glycoform of the humanized anti-α4β7 antibody.

In one embodiment, the composition comprises about 60% or less G0F glycoform of the humanized anti-α4β7 antibody.

In one embodiment, the composition comprises about 55% or less G0F glycoform of the humanized anti-α4β7 antibody.

In some embodiments, provided herein is a method of producing a composition having an increased amount of a G1F glycoform (as determined by Hydrophilic Interaction Chromatography (HILIC)) of the humanized anti-α4β7 antibody, said method comprising said culturing of a mammalian host cell in a production medium, and said adding of a supplement comprising uridine, manganese, and galactose to the production medium, thereby producing a composition having an increased amount of the G1F glycoform of the humanized anti-α4β7 antibody in comparison to a control mammalian host cell expressing the humanized anti-α4β7 antibody that is cultured in the absence of the supplement.

In one embodiment, the composition comprises at least about a 2-fold increase in the G1F glycoform of the humanized anti-α4β7 antibody in comparison to a control mammalian host cell expressing the humanized anti-α4β7 antibody that is cultured in the absence of the supplement.

In one embodiment, the composition comprises at least about a 3-fold increase in the G1F glycoform of the humanized anti-α4β7 antibody in comparison to a control mammalian host cell expressing the humanized anti-α4β7 antibody that is cultured in the absence of the supplement.

In some further embodiments, provided herein is a method of producing a composition having about 25% or more G1F glycoform (as determined by HILIC) of the humanized anti-α4β7 antibody, said method comprising said culturing of a mammalian host cell in a production medium, and said adding of a supplement comprising uridine, manganese, and galactose to the production medium, thereby producing a composition having about 25% or more G1F glycoform of the humanized anti-α4β7 antibody.

In one embodiment, the composition comprises about 30% or more G1F glycoform of the humanized anti-α4β7 antibody.

In some embodiments, provided herein is a method of producing a composition having an increased amount of a G2F glycoform (as determined by Hydrophilic Interaction Chromatography (HILIC)) of the humanized anti-α4β7 antibody, said method comprising said culturing of a mammalian host cell in a production medium, and said adding of a supplement comprising uridine, manganese, and galactose to the production medium, thereby producing a composition having an increased amount of the G2F glycoform of the humanized anti-α4β7 antibody in comparison to a control mammalian host cell expressing the humanized anti-α4β7 antibody that is cultured in the absence of the supplement.

In one embodiment, the composition comprises at least about a 3-fold increase in the G2F glycoform of the humanized anti-α4β7 antibody in comparison to a control mammalian host cell expressing the humanized anti-α4β7 antibody that is cultured in the absence of the supplement.

In one embodiment, the composition comprises at least about a 4-fold increase in the G2F glycoform of the humanized anti-α4β7 antibody in comparison to a control mammalian host cell expressing the humanized anti-α4β7 antibody that is cultured in the absence of the supplement.

In some embodiments, provided herein is a method of producing a composition having about 3% or more G2F glycoform (as determined by HILIC) of the humanized anti-α4β7 antibody, said method comprising said culturing of a mammalian host cell in a production medium, and said adding of a supplement comprising uridine, manganese, and galactose to the production medium, thereby producing a composition having about 3% or more G2F glycoform of the humanized anti-α4β7 antibody6.

In one embodiment, the composition comprises about 4% or more G2F glycoform of the humanized anti-α4β7 antibody.

In one embodiment, the supplement is added to the production medium or is added to a feed medium which is subsequently added to the production medium.

In one embodiment, the feed medium comprises aboutl5 to 100 mM uridine. In one embodiment, the feed medium comprises about 20 to 50 mM uridine. In one embodiment, the feed medium comprises about 1 to 40 mM uridine.

In one embodiment, the feed medium comprises about 0.02 to 0.3 mM manganese. In one embodiment, the feed medium comprises about 0.02 to 0.1 mM manganese. In one embodiment, the feed medium comprises about 0.001 to 0.1 mM manganese.

In one embodiment, the feed medium comprises 85 mM to 600 mM galactose. In one embodiment, the feed medium comprises 85 to 100 mM galactose. In one embodiment, the feed medium comprises 50 to 150 mM galactose.

In yet another embodiment, the production medium further comprises zinc. In one embodiment, the concentration of zinc in the feed medium is about 50 μM to 150 μM.

In one embodiment, the method further decreases the percentage of acidic species of the humanized anti-α4β7 antibody relative to the percentage of acidic species produced in a control mammalian host cell expressing the humanized anti-α4β7 antibody that is cultured in the absence of the supplement.

In one embodiment, the method further increases the percentage of main isoform species of the humanized anti-α4β7 antibody relative to the percentage of main isoform species produced in the absence of a feed medium comprising uridine, manganese, and galactose added to the production medium.

In one embodiment, the method is a fed batch method.

In one embodiment, the feed medium is added to the production medium beginning on about day four of the production phase. In one embodiment, the feed medium is added to the production medium daily beginning on about day 4 of the production phase.

In one embodiment, the methods disclosed herein are performed in a large scale bioreactor. In one embodiment, the large scale bioreactor is selected from the group consisting of a 200 liter (L) bioreactor, a 2000 L bioreactor, a 3000 L, and a 6000 L bioreactor.

In one embodiment, the production phase results in a titer of the humanized anti-α4β7 antibody of greater than 3 g/L. In one embodiment, the titer of the humanized anti-α4β7 antibody is about 3 to about 8 g/L. In one embodiment, the titer of the humanized anti-α4β7 antibody is about 5 to about 7 g/L.

In one embodiment, the mammalian host cell is a Chinese Hamster Ovary (CHO) cell. In one embodiment, the CHO cell is a GS-CHO cell.

In one embodiment, the production medium has a pH of about 6.8 to about 7.1.

In one embodiment, the humanized anti-α4β7 antibody comprises a heavy chain variable domain comprising an amino acid sequence as set forth in SEQ ID NO: 1, and comprises a light chain variable domain comprising an amino acid sequence as set forth in SEQ ID NO: 5.

In one embodiment, the anti-α4β7 antibody is vedolizumab.

In some embodiments, the method comprises harvesting and purification of the antibody. In some such embodiments, the purification comprises (i) purification steps that remove any cellular debris, unwanted proteins, salts, minerals or other undesirable elements, and (ii) purification of the antibody from contaminant soluble proteins and polypeptides. In certain embodiments, the method further comprises preparing a pharmaceutical formulation of the purified antibody which is suitable for human therapeutic use.

In particular embodiments, the pharmaceutical formulation is a liquid pharmaceutical formulation. In some such embodiments, the liquid pharmaceutical formulation is prepared by ultrafiltration/diafiltration.

In other embodiments, the pharmaceutical formulation is a lyophilized dry antibody formulation. In some such embodiments, the pharmaceutical formulation of the antibody is a dry antibody formulation lyophilized from a liquid pharmaceutical antibody formulation prepared by ultrafiltration/diafiltration following the purification.

In another aspect, provided herein is a cell culture comprising a host cell genetically engineered to express a humanized anti-α4β7 antibody, and a production medium supplemented with uridine, manganese, and galactose (UMG), wherein the humanized anti-α4β7 antibody is an IgG1 antibody and comprises a heavy chain variable region comprising the amino acid sequence set forth in SEQ ID NO:1, and a light chain variable region comprising the amino acid sequence set forth in SEQ ID NO: 5.

In some embodiments of the above aspect, the production medium comprises uridine at a concentration of about 15-100 mM, manganese at a concentration of about 20-200 nM, and galactose at a concentration of about 85-500 mM.

In some embodiments of the above aspect, the production medium comprises supplemented uridine at a concentration of about 1-7 mM, supplemented manganese at a concentration of about 2-15 μM, and supplemented galactose at a concentration of about 3-20 mM on the day of harvest. In some embodiments, the production medium further comprises supplemented zinc at a concentration of about 5-45 μM on the day of harvest.

In some embodiments of the above aspect, at the day of harvest, the production medium comprises uridine at a concentration of about 1-7 mM, manganese at a concentration of about 2-15 μM, and galactose at a concentration of about 3-20 mM. In some embodiments, at the day of harvest the production medium further comprises zinc at a concentration of about 5-45 μM.

In certain embodiments, the expressed humanized anti-α4β7 antibody has an isoform distribution comprising (a) 16% or less, 15% or less, 14% or less, 13% or less, or 12% or less basic isoform; and/or (b) at least 65%, at least 68%, at least 70%, at least 72%, or at least 75% major isoform.

In other embodiments, the expressed humanized anti-α4β7 antibody has a fucosylated N-glycan content comprising (a) 65% or less, 60% or less, or 55% or less G0F; (b) 25% or more, 27% or more, or 30% or more G1F; and/or (c) 2.5% or more, 3% or more, 3.5% or more, 4% or more, or 4.5% or more G2F.

In some embodiments of the above aspect, the expressed humanized anti-α4β7 antibody has a total fucosylated N-glycan content (G0F+G1F+G2F) of at least 92%, at least 93%, at least 94%, or at least 95%.

In other embodiments, the expressed humanized anti-α4β7 antibody has a total fucosylated N-glycan content (G0F+G1F+G2F) of 92-95%.

In alternative embodiments, the expressed humanized anti-α4β7 antibody has a total fucosylated N-glycan content (G0F+G1F+G2F) of 91-92%, 91-92.5%, or 91-93%.

In some embodiments of the above aspect, the cell culture further comprises zinc. In other embodiments, the cell culture further comprises arginine and/or lysine.

In some embodiments of the above aspect, the host cell is a CHO cell. In certain embodiments, the CHO cell is deficient in the gene encoding glutamine synthetase (GS).

In another aspect, the present disclosure provides a humanized anti-α4β7 antibody produced by the cell culture described hereinabove.

In yet another aspect, the present disclosure provides a composition comprising a humanized anti-α4β7 antibody, said method comprising culturing a mammalian host cell genetically engineered to express the humanized anti-α4β7 antibody in a first production medium having a first pH; and culturing the mammalian host cell in a second production medium having a second pH; wherein the second pH is lower than the first pH, and wherein the humanized anti-α4β7 antibody is an IgG1 antibody; comprises a heavy chain variable region comprising a CDR3 domain as set forth in SEQ ID NO: 4, a CDR2 domain as set forth in SEQ ID NO: 3, and a CDR1 domain as set forth in SEQ ID NO: 2; and comprises a light chain variable region comprising a CDR3 domain as set forth in SEQ ID NO: 8, a CDR2 domain as set forth in SEQ ID NO: 7, and a CDR1 domain as set forth in SEQ ID NO: 6.

In some embodiments of the aforementioned aspect, the second pH is 0.1 to 0.5 pH units lower than the first pH. In certain embodiments, the first pH is in the range of pH 6.8-7.2, and the second pH is in the range of pH 6.7-6.95.

In some embodiments, the mammalian host cell is cultured at the first pH for 120 hours or less. In certain embodiments, the mammalian host cell is cultured at the first pH for 85-110 hours. In other embodiments, the mammalian host cell is cultured at the first pH for 90-100 hours.

In some embodiments, the method further comprises harvesting the anti-α4β7 antibody from the second production medium. In certain embodiments, the anti-α4β7 antibody is harvested following culture of the mammalian host cell in the first production medium and the second production medium for a period of 13-15 days.

In some embodiments, the composition has an increased level of the anti-α4β7 antibody major isoform, relative to a control composition in which the mammalian host cell is cultured at the first pH without a pH shift.

In another aspect, the invention includes a composition comprising humanized anti-α4β7 antibodies produced using any one of the methods disclosed herein. In one embodiment, the methods disclosed herein provide a population of humanized anti-α4β7 antibodies having 92% or more, total asialo-, agalacto, core fucosylated biantennary glycan (G0F), asialo-, monogalacto, core fucosylated biantennary glycan (G1F), and/or asialo-, digalacto, core fucosylated biantennary glycan (G2F) glycosylation variants.

Additionally, the invention also comprises the following embodiments:
1. A method of producing a composition having a decreased amount of basic isoform (as determined by Cation Exchange Chromatography (CEX)) of a humanized anti-α4β7 antibody, said method comprising
   culturing a mammalian host cell in a production medium, and
   adding a supplement comprising uridine, manganese, and galactose to the production medium, thereby producing a composition having a decreased amount of basic isoform of the humanized anti-α4β7 antibody in comparison to a control mammalian host cell expressing the humanized anti-α4β7 antibody that is cultured in the absence of the supplement,
   wherein the mammalian host cell is genetically engineered to express a humanized anti-α4β7 antibody which is an IgG1 antibody; comprises a heavy chain variable region comprising a CDR3 domain as set forth in SEQ ID NO: 4, a CDR2 domain as set forth in SEQ ID NO: 3, and a CDR1 domain as set forth in SEQ ID NO: 2; and comprises a light chain variable region comprising a CDR3 domain as set forth in SEQ ID NO: 8, a CDR2 domain as set forth in SEQ ID NO: 7, and a CDR1 domain as set forth in SEQ ID NO: 6.
2. A method of producing a composition having about 16% or less basic isoform (as determined by CEX) of a humanized anti-α4β7 antibody, said method comprising
   culturing a mammalian host cell in a production medium, and
   adding a supplement comprising uridine, manganese, and galactose to the production medium, thereby producing a composition having about 16% or less basic isoform of the humanized anti-α4β7 antibody,

wherein the mammalian host cell is genetically engineered to express a humanized anti-α4β7 antibody which is an IgG1 antibody, comprises a heavy chain variable region comprising a CDR3 domain as set forth in SEQ ID NO: 4, a CDR2 domain as set forth in SEQ ID NO: 3, and a CDR1 domain as set forth in SEQ ID NO: 2; and comprises a light chain variable region comprising a CDR3 domain as set forth in SEQ ID NO: 8, a CDR2 domain as set forth in SEQ ID NO: 7, and a CDR1 domain as set forth in SEQ ID NO: 6.
3. The method of item 2, wherein the composition comprises about 14% or less basic isoform of the humanized anti-α4β7 antibody.
4. The method of item 2, wherein the composition comprises about 13% or less basic isoform of the humanized anti-α4β7 antibody.
5. The method of any one of items 1 to 4, wherein the supplement is added to the production medium or is added to a feed medium which is subsequently added to the production medium.
6. The method of item 5, wherein uridine is added to the feed medium at a final concentration of about 15 to 120 mM.
7. The method of item 6, wherein the uridine is added to the feed medium to a final concentration of about 20 to 70 mM uridine.
8. The method of item 5, wherein manganese is added to the feed medium at a final concentration of about 0.02 to 0.3 mM.
9. The method of item 8, wherein manganese is added to the feed medium to a final concentration of about 0.04 to 0.15 mM.
10. The method of item 5, wherein galactose is added to the feed medium to a final concentration of about 85 mM to 600 mM.
11. The method of item 10, wherein galactose is added to the feed medium to a final concentration of about 160 to 340 mM.
12. The method of any one of items 1-11, wherein the feed medium further comprises zinc.
13. The method of item 12, wherein the concentration of zinc in the feed medium is about 90 ₁.tM to 120 μM.
14. The method of any one of items 1-13, wherein the method further decreases the percentage of acidic species of the humanized anti-α4β7 antibody relative to the percentage of acidic species produced in a control mammalian host cell expressing the humanized anti-α4β7 antibody that is cultured in the absence of the supplement
15. The method of any one of items 1-14, wherein the method further increases the percentage of main isoform species of the humanized anti-α4β7 antibody relative to the percentage of main isoform species produced in the absence of a feed medium comprising uridine, manganese, and galactose added to the production medium.
16. The method of any one of items 1-15, wherein the method is a fed batch method.
17. The method of item 16, wherein the feed medium is added to the production medium beginning on about day four of the production phase.
18. A method of producing a composition having a decreased amount of basic isoform (as determined by Cation Exchange Chromatography (CEX)) of a humanized anti-α4β7 antibody, said method comprising culturing a mammalian host cell in a production medium comprising zinc, thereby producing a composition having a decreased amount of basic isoform of the humanized anti-α4β7 antibody in comparison to a control mammalian host cell expressing the humanized anti-α4β7 antibody that is cultured in the absence of zinc,
   wherein the mammalian host cell is genetically engineered to express a humanized anti-α4β7 antibody which is an IgG1 antibody; comprises a heavy chain variable region comprising a CDR3 domain as set forth in SEQ ID NO: 4, a CDR2 domain as set forth in SEQ ID NO: 3, and a CDR1 domain as set forth in SEQ ID NO: 2; and comprises a light chain variable region comprising a CDR3 domain as set forth in SEQ ID NO: 8, a CDR2 domain as set forth in SEQ ID NO: 7, and a CDR1 domain as set forth in SEQ ID NO: 6.
19. A method of producing a composition having about 16% or less basic isoform (as determined by CEX) of a humanized anti-α4β7 antibody, said method comprising culturing a mammalian host cell in a production medium comprising zinc, thereby producing a composition having about 16% or less basic isoform of the humanized anti-α4β7 antibody,
   wherein the mammalian host cell is genetically engineered to express a humanized anti-α4β7 antibody which is an IgG1 antibody, comprises a heavy chain variable region comprising a CDR3 domain as set forth in SEQ ID NO: 4, a CDR2 domain as set forth in SEQ ID NO: 3, and a CDR1 domain as set forth in SEQ ID NO: 2; and comprises a light chain variable region comprising a CDR3 domain as set forth in SEQ ID NO: 8, a CDR2 domain as set forth in SEQ ID NO: 7, and a CDR1 domain as set forth in SEQ ID NO: 6.
20. The method of item 19, wherein the composition comprises about 14% or less basic isoform of the humanized anti-α4β7 antibody.
21. The method of item 19, wherein the composition comprises about 13% or less basic isoform of the humanized anti-α4β7 antibody.
22. The method of any one of items 18 to 21, wherein the concentration of zinc in the production medium is 2 μM to 60 μM.
23. The method of any one of items 18 to 22, wherein the method comprises supplementing the production medium with zinc by adding a feed medium comprising zinc to the production medium.
24. The method of item 23, wherein the feed medium is added to the production medium beginning on about day four of the production phase.
25. The method of item 24, wherein the concentration of zinc in the feed medium is about 90 μM to 120 μM.
26. The method of any one of items 1-25, wherein the production medium comprises 5.0 to 8.8 g/L lysine and 3.0 to 12.0 g/L arginine.
27. The method of item 26, wherein the production medium comprises 4.5 to 5.5 g/L lysine.
28. The method of item 26, wherein the production medium comprises 5.5 to 8.8 g/L lysine.
29. The method of item 26, wherein the production medium comprises 5.4 to 7.4 g/L arginine.
30. The method of item 26, wherein the production medium comprises 7.4 to 12 g/L arginine.
31. A method of producing a composition comprising 2.5% or less HMW species (as determined by SEC) of a humanized anti-α4β7 antibody, said method comprising culturing a mammalian host cell in a production medium in a production phase, such that a composition comprising 2.5% or less HMW species (as determined by SEC) of the humanized anti-α4β7 antibody is produced,
   wherein the production medium has an average temperature of about 37 degrees Celsius, wherein the host cell is genetically engineered to express a humanized IgG1 anti-α4β7
      antibody, wherein the humanized anti-α4β7 comprises a heavy chain variable region comprising a CDR3 domain as set forth in SEQ ID NO: 4, a CDR2 domain as set forth in SEQ ID NO: 3, and a CDR1 domain as set forth in SEQ ID NO: 2; and comprises a light chain variable region comprising a CDR3 domain as set forth in SEQ ID NO: 8, a CDR2 domain as set forth in SEQ ID NO: 7, and a CDR1 domain as set forth in SEQ ID NO: 6.
32. A method of producing a composition comprising high levels of monomer (as determined by SEC) of a humanized anti-α4β7 antibody, said method comprising
   culturing a mammalian host cell in a growth medium in an expansion phase, wherein the mammalian host cell is genetically engineered to express a humanized anti-a4r37 antibody, and
   culturing the mammalian host cell in a production medium in a production phase, such that a composition comprising high levels of monomer (as determined by SEC) of the humanized anti-α4β7 antibody is produced,
   wherein the mammalian host cell is cultured at a temperature that is approximately the same in both the expansion phase and the production phase, and
   wherein the humanized anti-α4β7 antibody is an IgG1 antibody; comprises a heavy chain variable region comprising a CDR3 domain as set forth in SEQ ID NO: 4, a CDR2 domain as set forth in SEQ ID NO: 3, and a CDR1 domain as set forth in SEQ ID NO: 2; and comprises a light chain variable region comprising a CDR3 domain as set forth in SEQ ID NO: 8, a CDR2 domain as set forth in SEQ ID NO: 7, and a CDR1 domain as set forth in SEQ ID NO: 6.
33. The method of item 31 or 32, wherein the temperature is from 36 to 38 degrees Celsius.
34. The method of item 31 or 32, wherein the average temperature is from 36.5 to 37.5 degrees Celsius.
35. The method of item 31 or 32, wherein the temperature is an average temperature of about 37 degrees Celsius.
36. The method of any one of items 1-35, wherein the production medium has a temperature ranging from 36 to 38 degrees Celsius.
37. The method of item 36, wherein the temperature ranges from 36.5 to 37.5 degrees Celsius.
38. The method of item 36, wherein the temperature is an average temperature of about 37 degrees Celsius.
39. The method of any one of items 1-38, wherein the production medium has a pH ranging from 6.5 to 7.
40. The method of item 39, wherein the production medium has a pH ranging from 6.8 to 7.0.
41. The method of any one of items 1-40, wherein the production medium has a glucose level that is maintained at about 7 g/L or less a during the production phase.
42. The method of any one of items 1-41, wherein the production phase is 14 days or less.
43. The method of any one of items 1-42, wherein the production phase ranges from 10 days to 17 days.
44. The method of any one of items 1-43, which is performed in a large scale bioreactor.
45. The method of item 44, wherein the large scale bioreactor is selected from the group consisting of a 200 liter (L) bioreactor, a 2000 L bioreactor, a 3000L, and a 6000 L bioreactor.
46. The method of any one of items 1-45, wherein the production phase results in a titer of the humanized anti-α4β7 antibody of greater than 3 g/L.
47. The method of item 46, wherein the titer of the humanized anti-α4β7 antibody is about 3 to about 8 g/L.
48. The method of item 46, wherein the titer of the humanized anti-α4β7 antibody is about 5 to about 7 g/L.
49. The method of any one of items 1-48, wherein the mammalian host cell is a Chinese Hamster Ovary (CHO) cell.
50. The method of item 49, wherein the CHO cell is a GS-CHO cell.
51. The method of any one of items 1-50, wherein the humanized anti-α4β7 antibody comprises a heavy chain variable domain comprising an amino acid sequence as set forth in SEQ ID NO: 1, and comprises a light chain variable domain comprising an amino acid sequence as set forth in SEQ ID NO: 5.
52. The method of any one of items 1-50, wherein the humanized anti-α4β7 antibody is vedolizumab.
53. A composition comprising humanized anti-α4β7 antibodies produced using any one of the methods of items 1-52.
54. The composition of item 53, comprising a population of humanized anti-α4β7 antibodies having 92% or more, total asialo-, agalacto, core fucosylated biantennary glycan (G0F), asialo-, monogalacto, core fucosylated biantennary glycan (G1F), and/or asialo-, digalacto, core fucosylated biantennary glycan (G2F) glycosylation variants.
55. A method of producing a composition having a decreased amount of a G0F glycoform (as determined by Hydrophilic Interaction Chromatography (HILIC) of a humanized anti-α4β7 antibody, said method comprising
   culturing a mammalian host cell in a production medium, and
   adding a supplement comprising uridine, manganese, and galactose to the production medium, thereby producing a composition having a decreased amount of the G0F glycoform of the humanized anti-α4β7 antibody in comparison to a control mammalian host cell expressing the humanized anti-α4β7 antibody that is cultured in the absence of the supplement,
   wherein the mammalian host cell is genetically engineered to express a humanized anti-α4β7 antibody which is an IgG1 antibody; comprises a heavy chain variable region comprising a CDR3 domain as set forth in SEQ ID NO: 4, a CDR2 domain as set forth in SEQ ID NO: 3, and a CDR1 domain as set forth in SEQ ID NO: 2; and comprises a light chain variable region comprising a CDR3 domain as set forth in SEQ ID NO: 8, a CDR2 domain as set forth in SEQ ID NO: 7, and a CDR1 domain as set forth in SEQ ID NO: 6.
56. The method of item 55, wherein the composition comprises at least about a 15% decreased level of the G0F glycoform of the humanized anti-α4β7 antibody in comparison to a control mammalian host cell expressing the humanized anti-α4β7 antibody that is cultured in the absence of the supplement.
57. The method of item 55, wherein the composition comprises at least about a 20% decrease in the G0F glycoform of the humanized anti-α4β7 antibody in comparison to a control mammalian host cell expressing the humanized anti-α4β7 antibody that is cultured in the absence of the supplement.
58. A method of producing a composition having about 65% or less G0F glycoform (as determined by HILIC) of a humanized anti-α4β7 antibody, said method comprising
   culturing a mammalian host cell in a production medium, and
   adding a supplement comprising uridine, manganese, and galactose to the production medium, thereby producing a composition having about 65% or less G0F glycoform of the humanized anti-α4β7 antibody,
   wherein the mammalian host cell is genetically engineered to express a humanized anti-α4β7 antibody which is an IgG1 antibody, comprises a heavy chain variable region comprising a CDR3 domain as set forth in SEQ ID NO: 4, a CDR2 domain as set forth in SEQ ID NO: 3, and a CDR1 domain as set forth in SEQ ID NO: 2; and comprises a light chain variable region comprising a CDR3 domain as set forth in SEQ ID NO: 8, a CDR2 domain as set forth in SEQ ID NO: 7, and a CDR1 domain as set forth in SEQ ID NO: 6.
59. The method of item 58, wherein the composition comprises about 60% or less G0F glycoform of the humanized anti-α4β7 antibody.
60. The method of item 58, wherein the composition comprises about 55% or less G0F glycoform of the humanized anti-α4β7 antibody.
61. A method of producing a composition having an increased amount of a G1F glycoform (as determined by HILIC) of a humanized anti-α4β7 antibody, said method comprising
   culturing a mammalian host cell in a production medium, and
   adding a supplement comprising uridine, manganese, and galactose to the production medium, thereby producing a composition having an increased amount of the GIF glycoform of the humanized anti-α4β7 antibody in comparison to a control mammalian host cell expressing the humanized anti-α4β7 antibody that is cultured in the absence of the supplement,
   wherein the mammalian host cell is genetically engineered to express a humanized anti-α4β7 antibody which is an IgG1 antibody; comprises a heavy chain variable region comprising a CDR3 domain as set forth in SEQ ID NO: 4, a CDR2 domain as set forth in SEQ ID NO: 3, and a CDR1 domain as set forth in SEQ ID NO: 2; and comprises a light chain variable region comprising a CDR3 domain as set forth in SEQ ID NO: 8, a CDR2 domain as set forth in SEQ ID NO: 7, and a CDR1 domain as set forth in SEQ ID NO: 6.
62. The method of item 61, wherein the composition comprises at least about a 2-fold increase in the G1F glycoform of the humanized anti-α4β7 antibody in comparison to a control mammalian host cell expressing the humanized anti-α4β7 antibody that is cultured in the absence of the supplement.
63. The method of item 61, wherein the composition comprises at least about a 3-fold increase in the G1F glycoform of the humanized anti-α4β7 antibody in comparison to a control mammalian host cell expressing the humanized anti-α4β7 antibody that is cultured in the absence of the supplement.
64. A method of producing a composition having about 25% or more G1F glycoform (as determined by HILIC) of a humanized anti-α4β7 antibody, said method comprising
   culturing a mammalian host cell in a production medium, and
   adding a supplement comprising uridine, manganese, and galactose to the production medium, thereby producing a composition having about 25% or more G1F glycoform of the humanized anti-α4β7 antibody,
   wherein the mammalian host cell is genetically engineered to express a humanized anti-α4β7 antibody which is an IgG1 antibody, comprises a heavy chain variable region comprising a CDR3 domain as set forth in SEQ ID NO: 4, a CDR2 domain as set forth in SEQ ID NO: 3, and a CDR1 domain as set forth in SEQ ID NO: 2; and comprises a light chain variable region comprising a CDR3 domain as set forth in SEQ ID NO: 8, a CDR2 domain as set forth in SEQ ID NO: 7, and a CDR1 domain as set forth in SEQ ID NO: 6.
65. The method of item 64, wherein the composition comprises about 30% or more GIF glycoform of the humanized anti-α4β7 antibody.
66. A method of producing a composition having an increased amount of a G2F glycoform (as determined by HILIC) of a humanized anti-α4β7 antibody, said method comprising
   culturing a mammalian host cell in a production medium, and
   adding a supplement comprising uridine, manganese, and galactose to the production medium, thereby producing a composition having an increased amount of the G2F glycoform of the humanized anti-α4β7 antibody in comparison to a control mammalian host cell expressing the humanized anti-α4β7 antibody that is cultured in the absence of the supplement,
   wherein the mammalian host cell is genetically engineered to express a humanized anti-α4β7 antibody which is an IgG1 antibody; comprises a heavy chain variable region comprising a CDR3 domain as set forth in SEQ ID NO: 4, a CDR2 domain as set forth in SEQ ID NO: 3, and a CDR1 domain as set forth in SEQ ID NO: 2; and comprises a light chain variable region comprising a CDR3 domain as set forth in SEQ ID NO: 8, a CDR2 domain as set forth in SEQ ID NO: 7, and a CDR1 domain as set forth in SEQ ID NO: 6.
67. The method of item 66, wherein the composition comprises at least about a 3-fold increase in the G2F glycoform of the humanized anti-α4β7 antibody in comparison to a control mammalian host cell expressing the humanized anti-α4β7 antibody that is cultured in the absence of the supplement.
68. The method of item 66, wherein the composition comprises at least about a 4-fold increase in the G2F glycoform of the humanized anti-α4β7 antibody in comparison to a control mammalian host cell expressing the humanized anti-α4β7 antibody that is cultured in the absence of the supplement.
69. A method of producing a composition having about 3% or more G2F glycoform (as determined by HILIC) of a humanized anti-α4β7 antibody, said method comprising culturing a mammalian host cell in a production medium, and adding a supplement comprising uridine, manganese, and galactose to the production medium, thereby producing a composition having about 3% or more G2F glycoform of the humanized anti-α4β7 antibody, wherein the mammalian host cell is genetically engineered to express a humanized anti-α4β7 antibody which is an IgG1 antibody, comprises a heavy chain variable region comprising a CDR3 domain as set forth in SEQ ID NO: 4, a CDR2 domain as set forth in SEQ ID NO: 3, and a CDR1 domain as set forth in SEQ ID NO: 2; and comprises a light chain variable region comprising a CDR3 domain as set forth in SEQ ID NO: 8, a CDR2 domain as set forth in SEQ ID NO: 7, and a CDR1 domain as set forth in SEQ ID NO: 6.
70. The method of item 69, wherein the composition comprises about 4% or more G2F glycoform of the humanized anti-α4β7 antibody.
71. The method of any one of items 55-70, wherein the supplement is added to the production medium or is added to a feed medium which is subsequently added to the production medium.
72. The method of item 71, wherein the feed medium comprises aboutl5 to 100 mM uridine.
73. The method of item 72, wherein the feed medium comprises about 20 to 50 mM uridine.
74. The method of item 71, wherein the feed medium comprises about 0.02 to 0.3 mM manganese.
75. The method of item 74, wherein the feed medium comprises about 0.02 to 0.1 mM manganese.
76. The method of item 71, wherein the feed medium comprises 85 mM-600 mM galactose.
77. The method of claim 76, wherein the feed medium comprises 85 mM-100 mM galactose.
78. The method of any one of items 55-77, wherein the production medium further comprises zinc.
79. The method of item 78, wherein the concentration of zinc in the production medium is about 50 μM to 150 μM.
80. The method of any one of items 55-79, wherein the method further decreases the percentage of acidic species of the humanized anti-α4β7 antibody relative to the percentage of acidic species produced in a control mammalian host cell expressing the humanized anti-α4β7 antibody that is cultured in the absence of the supplement
81. The method of any one of items 55-80, wherein the method further increases the percentage of main isoform species of the humanized anti-α4β7 antibody relative to the percentage of main isoform species produced in the absence of a feed medium comprising uridine, manganese, and galactose added to the production medium.
82. The method of any one of items 55-81, wherein the method is a fed batch method.
83. The method of item 82, wherein the feed medium is added to the production medium beginning on about day four of the production phase.
84. The method of any one of items 1-83, wherein the production medium has a pH of about 6.8 to about 7.1.

### DESCRIPTION OF FIGURES

FIG. 1. Provides results from the prediction profiler based on experiments testing various culture conditions, including pH of the production cell culture, temperature, galactose Gal+ addition, addition of UMG, and feeding strategy conditions.

FIGS. 2A-2H graphically depicts results comparing the effects of uridine, galactose and manganese (UMG) supplementation (33xUMG, 50xUMG, and 66xUMG) and pH (pH 7.05 vs pH 6.85) on antibody titer (FIG. 2A), acidic species (FIG. 2B), basic species (FIG. 2C), percentage of major species (FIG. 2D), percentage of G0F species (FIG. 2E), percentage of G1F species (FIG. 2F), percentage of G2F species (FIG. 2G), and glycan species summation (FIG. 2H). Results without UMG supplementation (as indicated by the “+” symbol) are shown for comparison.

FIGS. 3A and 3B graphically depicts results comparing the effects of different arginine and lysine concentrations on the percentage of basic species (FIG. 3A) and antibody titer (FIG. 3B). The labels on the X-axis correspond to high (H), medium (M), or low (L) concentrations of lysine and arginine as outlined in Table 5.

FIGS. 4A-4C. Maximum desirability prediction results from JMP analysis of culture conditions with different levels of lysine and arginine (Low Lysine and low Arginine (LL)—FIG. 4A; Low lysine and medium arginine (LM)—FIG. 4B; Low lysine and high arginine (LH)—FIG. 4C). Concentrations corresponding to high (H), medium (M), or low (L) concentrations of lysine and arginine are outlined in Table 5.

FIGS. 5A-5H graphically depict time course data comparing the effects of zinc on antibody titer (FIG. 5A), percentage of basic species (FIG. 5B), percentage of acidic species (FIG. 5C), percentage of major species (FIG. 5D), percentage of G0F species (FIG. 5E), percentage of G1F species (FIG. 5F), percentage of G2F species (FIG. 5G), and glycan species summation (FIG. 5H) after 14, 15, 16, 17, and 18 culture days. The numbers on the x-axis correspond to the zinc concentrations outlined in Table 6.

FIGS. 6A-6E graphically depict time course data comparing the effects of zinc, culture days, and temperature (33° C., 35° C., and 37° C.) on the percentage of basic species (FIG. 6A), glycan species summation (FIG. 6B), aggregate (high molecular weight (HMW)) formation (FIG. 6C), titer (FIG. 6D), and acidic isoform (FIG. 6E). The solid black line in FIGS. 6A, 6C, and 6E represents the upper process criteria for each attribute, while the black line in FIG. 6B represents the lower acceptance criteria. The numbers on the x-axis correspond to the zinc concentrations outlined in Table 6.

FIGS. 7A-7D graphically depict time course data comparing the effects of days in vedolizumab culture on the percentage of acidic species (FIG. 7A), percentage of basic species (FIG. 7B), percentage of main species (FIG. 7C) and antibody titer (FIG. 7D) from two sets of experiments. Run 2 is represented by open circles, while Run 1 data points are represented by closed circles in FIGS. 7A-7D.

FIG. 8A-8B graphically depict the correlation between isoform distribution and pH shift parameters. FIG. 8A depicts the correlation between final cell culture pH (after a pH shift) and % acidic isoform species (left panel) or % major isoform (right panel). FIG. 8B depicts the correlation between the pH shift duration and % acidic isoform species (left panel) or % major isoform (right panel).

FIG. 9 depicts the structure of N-glycans that can be present in a population of an anti-α4≢7 antibody, such as vedolizumab. A key to the glycans is provided in the Figure.

### DETAILED DESCRIPTION OF THE INVENTION

### I. Definitions

In order that the present invention may be more readily understood, certain terms are first defined.

The cell surface molecule, “α4β7 integrin,” or “α4β7” (used interchangeably throughout) is a heterodimer of an α4 chain (CD49D, ITGA4) and a β7 chain (ITGB7). Human α4-integrin and β7-integrin genes GenBank (National Center for Biotechnology Information, Bethesda, Md.) RefSeq Accession numbers NM_000885 and NM_000889, respectively) are expressed by B and T lymphocytes, particularly memory CD4+ lymphocytes. Typical of many integrins, α4β7 can exist in either a resting or activated state. Ligands for α4β7 include vascular cell adhesion molecule (VCAM), fibronectin and mucosal addressin (MAdCAM (e.g., MAdCAM-1)). An antibody that binds to α4β7 integrin is referred to herein as an “anti-α4β7 antibody”.

As used herein, an antibody, or antigen-binding fragment thereof, that has “binding specificity for the α4β7 complex” binds to α4β7, but not to α4β1 or α_{E}B7. Vedolizumab is an example of an antibody that has binding specificity for the α4β7 complex.

The term “about” denotes that the thereafter following value is no exact value but is the center point of a range that is +/-5% of the value of the value. If the value is a relative value given in percentages the term “about” also denotes that the thereafter following value is no exact value but is the center point of a range that is +/−5% of the value, whereby the upper limit of the range cannot exceed a value of 100%.

As used herein, the terms “aggregate” or “aggregates” refer to the association of two or more antibodies or antibody fragments. For example, an aggregate can be a dimer, trimer, tetramer, or a multimer greater than a tetramer, of antibodies and/or antibody fragments. Antibody aggregates can be soluble or insoluble. The association between the aggregated molecules may be either covalent or non-covalent without respect to the mechanism by which they are associated. The association may be direct between the aggregated molecules or indirect through other molecules that link them together.

Examples of the latter include, but are not limited to disulfide linkages with other proteins, hydrophobic associations with lipids, charge associations with DNA, affinity associations with leached protein A, or mixed mode associations with multiple components. Aggregates can be irreversibly formed either during protein expression in cell culture, during protein purification in downstream processing, or during storage of the drug product. The presence of aggregates in a solution can be determined using, for example, size exclusion chromatography (SEC) (e.g., SEC with UV detection, SEC with light scattering detection (SEC-LSD)), field flow fractionation, analytical ultracentrifugation sedimentation velocity, or capillary electrophoresis-sodium dodecyl sulfate (CE-SDS, reduced and non-reduced).

The term “antibody” as used herein, is intended to refer to an immunoglobulin molecule comprised of four polypeptide chains, two heavy (H) chains and two light (L) chains inter-connected by disulfide bonds. Each heavy chain is comprised of a heavy chain variable region (abbreviated herein as HCVR or VH) and a heavy chain constant region (CH). The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDRs), interspersed with regions that are more conserved, termed framework regions (FR). Each

VH and VL is composed of three CDRs and four FRs, arranged from amino-terminus to carboxy-terminus in the following order: FR1, CDR1, FR2, CDR2, FR3, CDR3, FR4. In some embodiments, the antibody has a fragment crystallizable (Fc) region. In certain embodiments, the antibody is an IgG1 isotype and has a kappa light chain.

The terms “charged species,” “charged isoforms,” or “charged isoform species,” as used herein, refer to variants of an antibody, or antigen binding portion thereof (e.g., vedolizumab, or an antigen binding portion thereof) which are characterized by an overall charge that is distinct from the main species of the antibody, or antigen binding portion thereof, Charged isoform species of an antibody or antigen binding portion thereof can be detected by various methods known in the art, such as cation exchange chromatography (CEX), e.g., cation exchange-high performance liquid chromatography (CEX-HPLC), CEX-mass spectrometry, or isoelectric focusing. For example, in general, when an antibody preparation is resolved using CEX, CEX-HPLC, or CEX-mass spectrometry, the majority of the antibody elutes from the CEX resin with a retention time that is characteristic of the predominant (main) isoform of that antibody. This can be visualized by plotting the amount of antibody eluted from the resin as a function of the retention time on the CEX resin. When visualized in this manner, the main isoform of an antibody or antigen binding portion thereof is the fraction of the antibody or antigen binding portion thereof which elutes from the CEX resin within the largest peak. Using this method, charged isoform species can be identified by having a retention time that differs from that of the main isoform. For example, when charged isoform species are detected by CEX, CEX-HPLC, or CEX-mass spectrometry, acidic isoform species can elute from the resin with a shorter retention time than the main isoform of the antibody, or antigen binding portion thereof, and basic isoform species can elute from the resin with a longer retention time than the main isoform of the antibody, or antigen binding portion thereof.

The terms “acidic species” or “acidic isoform species,” as used herein, refer to variants of an antibody, or antigen binding portion thereof (e.g., vedolizumab, or an antigen binding portion thereof) which are characterized by an overall acidic charge. Acidic species of an antibody or antigen binding portion thereof can be detected by various methods known in the art, such as cation exchange chromatography (CEX), e.g., cation exchange-high performance liquid chromatography (CEX-HPLC), CEX-mass spectrometry, or isoelectric focusing. In general, acidic species of an antibody, or antigen binding portion thereof, elute from a CEX resin with a shorter retention time than the main isoform of the antibody, or antigen binding portion thereof. Acidic species of an antibody may include, but are not limited to, charge variants, structural variants, and/or fragmentation variants. In some embodiments, a composition comprising an antibody, or antigen binding portion thereof, can comprise more than one type of acidic isoform species. In some embodiments, multiple acidic isoform species can be identified based on differences in retention time during CEX-HPLC separation. For example, when a composition comprising an antibody, e.g., vedolizumab, is analyzed using CEX, one or more acidic isoform peaks may be identified, each representing one or more acidic isoform species of the antibody. The terms “basic species” or “basic isoform species”, as used herein, refer to variants of an antibody, or antigen binding portion thereof, e.g., vedolizumab, which are characterized by an overall basic charge. Basic species of an antibody or antigen binding portion thereof can be detected by various methods known in the art, such as cation exchange chromatography (CEX), e.g., cation exchange-high performance liquid chromatography (CEX-HPLC), CEX-mass spectrometry, or isoelectric focusing. In general, basic species of an antibody, or antigen binding portion thereof, elute from a CEX resin with a longer retention time than the main isoform of the antibody, or antigen binding portion thereof. Basic species of an antibody may include, but are not limited to, charge variants, structural variants, and/or fragmentation variants. In some embodiments, a composition comprising an antibody, or antigen binding portion thereof, can comprise more than one type of basic isoform species. In some embodiments, multiple basic isoform species can be identified based on differences in retention time during CEX-HPLC separation. For example, when a composition comprising an antibody, e.g., vedolizumab, is analyzed using CEX, one or more basic isoform peaks may be identified, each representing one or more basic isoform species of the antibody. In one embodiment, a basic isoform of vedolizumab is vedolizumab having a carboxyl-terminal lysine (C-Lys). Host cell impurities, or other impurities that are not related to the antibody, or antigen binding portion thereof, by primary sequence, are not considered “basic species” or “basic isoform species” of the antibody, or antigen binding portion thereof.

A “CDR” or “complementarity determining region” is a region of hypervariability interspersed within regions that are more conserved, termed “framework regions” (FR).

As used herein, the term “antigen binding fragment” or “antigen binding portion” of an antibody refers to Fab, Fab', F(ab')2, and Fv fragments, single chain antibodies, functional heavy chain antibodies (nanobodies), as well as any portion of an antibody having specificity toward at least one desired epitope, that competes with the intact antibody for specific binding (e.g., an isolated portion of a complementarity determining region having sufficient framework sequences so as to bind specifically to an epitope). Antigen binding fragments can be produced by recombinant techniques, or by enzymatic or chemical cleavage of an antibody.

As used herein, the term “humanized antibody” refers to an antibody that is derived from a non-human antibody (e.g., murine) that retains or substantially retains the antigen-binding properties of the parent antibody but is less immunogenic in humans.

A polypeptide, such as an antibody, produced by a recombinant mammalian host cell line using cell culture methods is referred to as a “recombinant polypeptide”, “protein” or, with respect to an antibody, a “recombinant antibody”. The expressed protein may be produced intracellularly or secreted into the culture medium from which it can be recovered and/or collected. In one embodiment, a recombinant antibody is a recombinant anti-α4β7 antibody, e.g., an antibody that binding specificity for the α4β7 complex, such as vedolizumab. As the methods and compositions described herein relate to compositions and cell culture methods for producing a recombinant antibody, unless otherwise specified, the term “antibody” is used interchangeably herein with the term “recombinant antibody”.

The term “recombinant host cell” or “host cell” refers to a cell that has been genetically engineered to express a recombinant polypeptide, e.g., antibody. In one embodiment, a recombinant host cell comprises an expression vector comprising a nucleic acid encoding an antibody heavy chain, a light chain, or both. It should be understood that the term “host cell” is intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein. Further, it should be understood that unless specified otherwise, where the term “cell” is used, e.g., host cell, mammalian cell, or mammalian host cell, the term is intended to include a population of cells.

The term “cell culture process”, as used herein, refers collectively to the cell culture phases associated with recombinant polypeptide, e.g., antibody, production. The term “cell culture process” generally refers to the process by which cells are grown or maintained under controlled conditions. The cell culture process may take place in vitro or ex vivo. In some embodiments, a cell culture process has both an expansion phase and a production phase. In some embodiments, the expansion and production phases are separated by a transition or shift phase. “Culturing” a cell refers to contacting a cell with a cell culture medium under conditions suitable for growing or maintaining the cell. Cell culture, in certain embodiments, refers to methods for generating or maintaining a population of host cells capable of producing a recombinant polypeptide of interest, e.g., an anti-α4β7 antibody. For example, once an expression vector has been incorporated into an appropriate mammalian host cell, e.g., a Chinese Hamster Ovary (CHO) host cell, the host can be cultured under conditions suitable for expression of the relevant nucleotide coding sequences. A “cell culture” can also refer to a solution containing cells.

The terms “medium” and “cell culture medium” (plural, “media”) refer to a nutrient source used for growing or maintaining cells. As is understood by a person of skill in the art, the nutrient source may contain components required by the cell for growth and/or survival or may contain components that aid in cell growth and/or survival. Vitamins, essential or non-essential amino acids (e.g., cysteine and cystine), and trace elements (e.g., copper) are examples of medium components. Examples of cell culture media include growth medium and production medium.

A cell culture medium may also be supplemented, e.g., with a “medium supplement” or “supplement” with any one or more of a component that aids the cell culture process, e.g., by increasing recombinant polypeptide production or improving cell viability. In one embodiment, a supplement is not formulated with the cell culture medium, e.g., not formulated with production medium or feed medium. A supplement may be prepared in concentrated form where its combination with a feed solution or culture medium results in a final lower concentration of the supplemented component. A supplement may comprise one or more components already present in the starting, e.g., stock or base, medium, and/or a supplement may comprise one or more components new to the medium. In one embodiment, a supplement is added to a feed solution.

A supplement may affect a particular aspect of a cell culture, e.g., improve cell growth or increase recombinant polypeptide production, depending on the cell type, the growth format and the product (protein of interest) characteristics. Examples of substances that may be added by a supplement include, but are not limited to, one or more of a trace element, one or more of a hormone, one or more of an amino acid, one or more of a vitamin, one or more of a fatty acid, one or more of a nonionic detergent, one or more of a nucleotide, and/or one or more of a sugar. In some embodiments, a supplement comprises insulin, plant hydrolysates and/or animal hydrolysates. One or more supplements may be added at one stage of the cell culture process or at multiple stages thereof.

Cell culture medium and/or supplement may be “defined” or “undefined” to particular degrees, in that the sources of variability maybe known or unknown, based on the nature of the component(s), e.g., whether supplied as a known chemical composition, such as one or more of an element, an inorganic salt or organic ion or sugar, or whether supplied as a complex component, such as a mixture, e.g., a hydrolysate. The presence of complex components, such as proteins or hydrolysates, in the culture medium reduces the degree of definition.

The terms “growth phase”, “growth stage”, “expansion phase” and “expansion stage”, used interchangeably herein, refer to the period during which cultured host cells are rapidly dividing and increasing in number. During the expansion phase, cells may be generally cultured in a growth medium (or expansion medium) and under conditions designed to maximize cell proliferation. The growth phase can precede the production phase in time, e.g., in a batch culture, whereby the two phases may (or may not) be separated by a transition phase.

The term “production phase” or “production stage” as used herein, refers to a period during which host cells are producing maximal amounts of a recombinant polypeptide, such as a recombinant antibody. A production phase is typically characterized by less cell division than during an expansion phase, and may also include the use of medium and culture conditions designed to maximize polypeptide production.

The term “growth medium” refers to a cell culture medium that favors the growth, i.e., increase in number, of cultured cells and is used during the growth or expansion phase of the cell culturing process.

A “production medium” is a cell culture medium that favors the production of a recombinant polypeptide, e.g., antibody, of interest, e.g., an anti-α4β7 antibody.

A “feed solution” or a “feed medium”, as used herein, refers to a cell culture medium that is added to a cell culture in a growth or production medium in order to improve or maintain an aspect of the protein being produced by the cells in the growth or production medium. For example, a feed solution may be added to maintain a certain protein titer level being produced by the cells. Feed solutions are known in the art. In one embodiment, a feed solution is supplemented with additional nutrients identified as being beneficial to production of a protein from mammalian cells.

It will be appreciated that growth can also occur in a production medium and that production can take place in a growth medium, such that the growth and the production medium may be identical. In one embodiment, however, a production medium is chosen that favors the production of a polypeptide of interest to a greater extent than if the growth medium were employed.

The term “batch culture”, as used herein, refers to a culture in which all components for cell culturing (including the cells and all culture nutrients) are supplied to the culturing vessel at the start of the culturing process.

The term “fed batch cell culture,” as used herein, refers to a batch culture wherein the cells and culture medium are supplied to the culturing vessel initially, and additional supplements, e.g., nutrients, are fed (via a feed solution), continuously or in discrete increments to the culture during the culturing process, with or without periodic cell and/or product harvest before termination of culture.

The term “perfusion culture”, as used herein, refers to a culture in which the cells and supplements are supplied to the culturing vessel at the start of the culturing process and an additional supplement(s) are fed continuously to the culture, while the product is harvested continuously from the medium during the culturing process.

The term “vector,” as used herein, is intended to refer to a nucleic acid molecule capable of transporting another nucleic acid to which it has been linked. One type of vector is a “plasmid,” which refers to a circular double stranded DNA into which additional DNA segments may be ligated. Another type of vector is a phage vector. Another type of vector is a viral vector, wherein additional DNA segments may be ligated into a viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors,” or simply, “expression vectors.” In general, expression vectors of utility in recombinant DNA techniques are often in the form of plasmids.

A “nucleic acid” refers to a polymer of nucleotides of any length, and includes DNA and RNA. The nucleotides can be deoxyribonucleotides, ribonucleotides, modified nucleotides or bases, and/or their analogs, or any substrate that can be incorporated into a polymer by DNA or RNA polymerase, or by a synthetic reaction. A polynucleotide may comprise modified nucleotides, such as methylated nucleotides and their analogs. If present, modification to the nucleotide structure may be imparted before or after assembly of the polymer.

An “isolated nucleic acid” means and encompasses a non-naturally occurring, recombinant or a naturally occurring sequence outside of or separated from its usual context. An isolated nucleic acid molecule is other than in the form or setting in which it is found in nature. Isolated nucleic acid molecules therefore are distinguished from the nucleic acid molecule as it exists in natural cells. However, an isolated nucleic acid molecule includes a nucleic acid molecule contained in cells that ordinarily express the protein where, for example, the nucleic acid molecule is in a chromosomal location different from that of natural cells.

As used herein, “purified” (or “isolated”) refers to a nucleic acid molecule (e.g., a polynucleotide) or an amino acid molecule (e.g., a polypeptide or protein) that is substantially free of other components. In some embodiments, a purified polynucleotide or purified polypeptide is removed or separated from other components present in the environment in which it is produced. For example, an isolated polypeptide is one that is separated from other components of a cell in which it was produced (e.g., the endoplasmic reticulum or cytoplasmic proteins and RNA). An isolated polynucleotide is one that is separated from other nuclear components (e.g., histones) and/or from upstream or downstream nucleic acid sequences.

The term “culturing vessel” refers to a container used for culturing cells. The culturing vessel can be of any size so long as it is useful for the culturing of cells.

As used herein, the term “inoculation” or “seeding” refers to the addition of cells to a medium to begin the culture or to the process of providing a cell culture to a bioreactor or another vessel for culturing. The cells may have been propagated previously in another bioreactor or vessel. Alternatively, the cells may have been frozen and thawed immediately prior to providing them to the bioreactor or vessel. The term refers to any number of cells, including a single cell.

The term “titer” as used herein refers to the total amount of recombinantly expressed polypeptide, e.g., antibody, produced by a cell culture divided by a given amount of medium volume. Titer is typically expressed in units of milligrams of antibody per milliliter or grams per liter of medium. Titer can be expressed or assessed in terms of a relative measurement, such as a percentage increase in titer as compared to obtaining the protein product under different culture conditions.

The term “harvest,” as used herein, e.g., for an expressed protein that is secreted from the host cells, refers to the separation of the cell culture medium (containing the expressed protein of interest) from the cells and cellular debris of the cell culture. (Harvesting for a protein which is not secreted, would collect the cells and discard the medium.) The culture medium that contains the protein of interest is referred to as “conditioned medium.” Harvesting is performed using any of several techniques, including, but not limited to, centrifugation, microfiltration, depth filtration and filtration through absolute pore size membranes. Following the harvest, subsequent steps to isolate the desired protein, e.g., from the supernatant or from the cells, including clarification, are generally considered to be purification steps.

The term, “clarified harvest” refers to a liquid material derived from conditioned medium, containing the recombinant polypeptide of interest, for example, an anti-α4β7 antibody. A clarified harvest is obtained from a cell culture medium that has undergone one or more process steps to separate the polypeptide of interest from cells and cellular debris of the cell culture and/or to remove finer solid particles and particulate impurities from the liquid. Examples of such separation techniques include, but are not limited to, settling, flocculation, centrifugation, and/or filtration.

As used herein, the term “upstream process,” in the context of recombinant polypeptide, e.g., antibody, preparation, refers to activities involving the production and collection of polypeptides (e.g. antibodies) from cells (e.g., during cell culture of a protein, e.g., antibody, of interest).

As used herein, the term “downstream process” refers to one or more techniques used after the upstream process to purify the protein, e.g., antibody, of interest. For example, downstream process includes purification of the protein product, using, for example, affinity chromatography, including Protein A affinity chromatography, size exclusion chromatography, ion exchange chromatography, such as anion or cation exchange chromatography, hydrophobic interaction chromatography (HIC), or displacement chromatography.

As used herein, the term “glycosylation profile” refers to a composite of the species of post-translational modifications comprising oligosaccharides. In relation to an anti-α4β7 antibody, a glycosylation profile describes N-linked glycosylation in the Fc region of the antibody. In relation to vedolizumab, the glycosylation profile refers to glycosylated species linked to asparagine 301 of the heavy chain, SEQ ID NO:13.

### II. Methods and Compositions of the Invention

Provided herein are methods and compositions for producing an anti-α4β7 antibody, such as vedolizumab, in a mammalian, e.g., non-human, cell culture. The invention is based, at least in part, on cell culture parameters that can be used to achieve high anti-α4β7 antibody titer levels, i.e., greater than 1 g/L, e.g., 3-10 g/L, 4-8 g/L or 5-7 g/L, in mammalian cell culture. Also described herein are methods and compositions for achieving reduced levels of basic isoforms of an anti-α4β7 antibody, such as vedolizumab; methods and compositions for achieving low aggregate levels of an anti-α4β7 antibody, such as vedolizumab; and methods and compositions for achieving particular glycan forms of an anti-α4β7 antibody, such as vedolizumab. Also provided herein are compositions comprising an anti-α4β7 antibody, such as vedolizumab, having a reduced level of basic isoform species; a low level of high molecular weight aggregates; and/or particular glycan forms.

In particular, the methods and compositions disclosed herein may be used to produce the anti-α4β7 antibody vedolizumab, or antibodies having antigen binding regions of vedolizumab. Vedolizumab is also known by its trade name ENTYVIO® (Takeda Pharmaceuticals, Inc.). Vedolizumab is a humanized antibody that comprises mutated human IgG1 framework regions and antigen-binding CDRs from the murine antibody Act-1 (which is described in U.S. Pat. No. 7,147,851, incorporated by reference herein).

Vedolizumab specifically binds to the α4β7 integrin and blocks the interaction of α4β7 integrin with mucosal addressin cell adhesion molecule-1 (MAdCAM-1) and fibronectin and inhibits the migration of memory T-lymphocytes across the endothelium into inflamed gastrointestinal parenchymal tissue. Vedolizumab does not bind to or inhibit function of the α4β1 and αEβ7 integrins and does not antagonize the interaction of α4 integrins with vascular cell adhesion molecule-1 (VCAM-1).

The α4β7 integrin is expressed on the surface of a discrete subset of memory T-lymphocytes that preferentially migrate into the gastrointestinal tract. MAdCAM-1 is expressed on gut endothelial cells and plays a critical role in the homing of T-lymphocytes to gut lymph tissue. The interaction of the α4β7 integrin with MAdCAM-1 has been implicated as an important contributor to mucosal inflammation, such as the chronic inflammation that is a hallmark of ulcerative colitis and Crohn's disease. Vedolizumab may be used to treat inflammatory bowel disease, including Crohn's disease and ulcerative colitis, HIV, pouchitis, including chronic pouchitis, fistulizing Crohn's disease, graft versus host disease and celiac disease.

The heavy chain variable region of vedolizumab is provided in SEQ ID NO:1, and the light chain variable region of vedolizumab is provided in SEQ ID NO:5. Vedolizumab comprises a heavy chain variable region comprising a CDR1 described in SEQ ID NO:2, a

CDR2 described in SEQ ID NO:3, and a CDR3 described in SEQ ID NO:4. Vedolizumab comprises a light chain variable region comprising a CDR1 described in SEQ ID NO:6, a CDR2 described in SEQ ID NO:7 and CDR3 described in SEQ ID NO:8. A nucleic acid sequence encoding the light chain variable region is set forth in SEQ ID NO:9. A nucleic acid sequence encoding the heavy chain variable region is set forth in SEQ ID NO:10. A full length nucleic acid sequence encoding the light chain of vedolizumab is set forth as SEQ ID NO:11 and a full length nucleic acid sequence encoding the heavy chain of vedolizumab is set forth as SEQ ID NO:12. Nucleic acid sequences encoding vedolizumab are also described in U.S. Patent Publication No. 2010/0297699, the entire contents of which are incorporated herein. Vedolizumab and the sequences of vedolizumab are further described in U.S. Patent Publication No. 2014/0341885 and U.S. Patent Publication No. 2014/0377251, the entire contents of each which are expressly incorporated herein by reference in their entireties.

The methods and compositions provided herein are useful for producing an anti-α4β7 antibody, particularly vedolizumab or an antibody having the binding regions, i.e., CDRs or variable regions, of vedolizumab, or an antigen-binding fragment of an anti-α4β7 antibody in mammalian cells.

The methods and compositions disclosed herein relate to a mammalian cell culture process. Mammalian cells have become the dominant system for the production of mammalian proteins for clinical, e.g., human therapeutic, applications, primarily due to their ability to produce properly folded and assembled heterologous proteins, and their capacity for post-translational modifications, such as modifications similar to those made by human cells. Chinese hamster ovary (CHO) cells, and cell lines obtained from various other mammalian sources, such as, for example, mouse myeloma (NS0), baby hamster kidney (BHK), human embryonic kidney (HEK-293) and human retinal cells have been approved by regulatory agencies for the production of biopharmaceutical products, including therapeutic antibodies. Of these, CHO cells are among the most commonly used industrial hosts, which are widely employed for the production of heterologous proteins. Thus, methods for the large-scale production of antibodies in CHO cells, including dihydrofolate reductase negative (DHFR−) or glutamine synthase negative (GS−) CHO cells, are well known in the art (see, e.g. Trill et al., Curr. Opin. Biotechnol. 6(5):553-60 (1995), Birch and Racher, Adv. Drug Delivery Reviews 58:671-685 (2006) and U.S. Pat. No. 6,610,516). Examples of CHO cell lines suitable for use in the compositions and methods provided herein include, but are not limited to, GS-CHO, CHO-K1 DUX B11 and DP-12 CHO cells. CHO cells suitable for use in the compositions and methods provided herein have also been described in the following documents: U.S. Pat. Nos. 4,766,075; 4,853,330; 5,185,259; 5,122,464; 5,591,639; 5,879,936; Lubiniecki et al., in Advances in Animal Cell Biology and Technology for Bioprocesses, Spier et al., eds. (1989), pp. 442-451. Known CHO derivatives suitable for use herein include, for example, CHO/-DHFR (Urlaub and Chasin. Proc. Natl. Acad. Sci. USA, 77: 4216 (1980)), CHO-K1 DUX B11 (Simonsen and Levinson, Proc. Natl. Acad. Sci. USA 80: 2495-2499(1983); Urlaub and Chasin, supra), and DP-12 CHO cells (EP 307,247 published Mar. 15, 1989, or U.S. Pat. No. 5,721,121).

Other examples of suitable mammalian cell lines include monkey kidney CVI line transformed by SV40 (COS-7, ATCC™ CRL 1651); human embryonic kidney line 293S (Graham et al., J. Gen. Virolo., 36:59 (1977)); baby hamster kidney cells (BHK, ATCC™ CCL 10); mouse sertoli cells (TM4, Mather, Biol. Reprod., 23:243 (1980)); monkey kidney cells (CVI-76, ATCC™ CCL 70); African green monkey kidney cells (VERO-76, ATCC™ CRL-1587); human cervical carcinoma cells (HELA, ATCC™ CCL 2); canine kidney cells (MDCK, ATCC™ CCL 34); buffalo rat liver cells (BRL 3A, ATCC® CRL 1442); human lung cells (W138, ATCC™ CCL 75); human liver cells (Hep G2. HB 8065); mouse mammary tumor cells (MMT 060562, ATCCV CCL 51); rat hepatoma cells (HTC, MI.54, Baumann et al., J. Cell Biol., 85:1 (1980)), 3T3 cells; 293T cells (Pear, W. S., et al., Proc. Natl. Acad. Sci. U.S.A., 90:8392-8396 (1993)); NS0 cells (Sato et al. Tissue Culture Association, 24:1223 (1988)); SP2/0 (Sato et al. J. Exp. Med., 165:1761 (1987)); and TR-1 cells (Mather et al., Annals N.Y. Acad. Sci., 383:44 (1982)) and hybridoma cell lines.

While many host cell types are capable of producing an encoded recombinant polypeptide, a product encoded by a particular nucleic acid produced in one host cell may differ from the product encoded by that nucleic acid in another host cell. The difference may be in one or more biochemical characteristics. Examples of biochemical characteristics include fundamental protein structure, such as primary, secondary or tertiary structure, or post-translational modifications, such as signal peptide processing, glycosylation, N-terminal acetylation, lipidation, or phosphorylation. The particular differences may depend on the enzymatic machinery of the cell and/or on the culture medium or growth conditions. For a recombinant, therapeutic antibody, changes in biochemical characteristics may affect one or more antibody features such as binding capacity, antibody effector functions, immunogenicity, clearance, solubility, or storage stability.

In some embodiments, products with differences from a reference product may be reduced or eliminated by purification, e.g., downstream process technology. In other embodiments, products with differences from a reference product, may be reduced or eliminated by controlling enzymatic machinery of the cell, e.g., upstream process technology. In some embodiments, controlling enzymatic machinery of the cells includes mutation of the cell to recombinantly modify its genetic background, e.g., mutate or alter the expression of an enzyme. In some embodiments, controlling enzymatic machinery of the cells includes controlling ingredients in culture medium, such as providing a particular culture medium or adding one or more supplements. In some embodiments, controlling enzymatic machinery of the cells includes maintenance or adjustment of growth conditions such as temperature, pH or atmospheric gas.

Methods of producing an anti-α4β7 antibody, such as vedolizumab, have been described (see e.g., U.S. Pat. No. 7,402,410 and U.S. Patent Application Publication No. 20070122404). In these publications, certain characteristics of the antibody were described, e.g., binding affinity, effector functions, and biochemical characteristics, such as charge profile, molecular weight and glycosylation pattern. The antibody had certain characteristics when cultured in NSO cells which changed when cultured in CHO cells. Characteristics of a recombinant protein, e.g., antibody, also can change when changing to different variants of CHO cells, for example changing from DHFR- cells to GS-cells. Described herein are methods and medium compositions which control for these variations, e.g., in the production of a recombinant protein, e.g., antibody, and limit or minimize the change of characteristics when expressing an antibody, e.g., vedolizumab, in GS-CHO cells (also referred to herein as simply “GS-CHO” cells). In certain embodiments, described herein are methods and compositions for producing an anti-α4β7 antibody, such as vedolizumab, in GS-CHO cells.

Examples of characteristics that may change when producing an anti-α4β7 antibody, such as vedolizumab, in cell culture include its charge profile, glycosylation profile and high molecular weight (HMW) impurity species. Culture conditions such as temperature, pH, shear stress, dissolved oxygen and medium composition may contribute to the changed characteristics. The charge of the enzyme may change from variations in the presence or absence of C-terminal lysine, N-terminal pyroglutamic acid or sialic acid, and/or variations in deamidation or oxidation. The glycosylation profile may change from variations such as the presence or absence of sialic acid or terminal galactose, processing of high mannose species (see Hossler et al. (2009) Glycobiology 19:936-949). Media supplements may control such variations.

In some embodiments, the characteristics of an anti-α4β7 antibody produced in cell culture, including but not limited to charge variation, glycan variation, and aggregate content, can be controlled by modulating the amount of a sugar (e.g., galactose), a metal cofactor (e.g., manganese), and/or a nucleoside (e.g., uridine) in the culture medium, e.g., the production phase medium. In some embodiments, the characteristics of an anti-α4β7 antibody produced in cell culture, including but not limited to charge variation, glycan variation, and aggregate content, can be controlled by modulating the amount of lysine and arginine in the culture medium, e.g., the production phase medium. In some embodiments, the characteristics of an anti-α4β7 antibody produced in cell culture, including but not limited to charge variation, glycan variation, and aggregate content, can be controlled by modulating the amount of zinc used in the culture medium, e.g., the production phase medium. Further, a temperature shift may be employed during production. While it is known in the art that a temperature shift may prove beneficial for antibody production (Moore et al. (1997) Cytotechnology 23:47-54), provided herein are methods based on maintaining a cell culture temperature, i.e., where the culture conditions do not include a substantial shift, e.g., more than 1 degree above or below 37 degrees Celsius.

A. Supplementation with Zinc

In some embodiments, provided herein are methods and compositions for the production of a humanized anti-α4β7 antibody, e.g., vedolizumab, or an antigen binding portion thereof, in a CHO cell culture supplemented during the production phase with zinc. In some embodiments zinc is used as a medium supplement for controlling charge variation of an anti-α4β7 antibody in a CHO cell culture. In other embodiments, zinc is used as a medium supplement for controlling the level of high molecular weight (HMW) aggregates in a preparation of an anti-α4β7 antibody produced in a CHO cell culture. The metal ion may be in the form of a hydrochloride salt, a sulfate salt, a nitrate salt, a bromide salt, an acetate salt, a stearate salt, a citrate salt, a phosphate salt. The medium supplement may be provided in concentrated form to the culture together with the feed, at the beginning of the batch, during expansion phase, or in the production phase. The medium supplement may be provided in concentrated form to a feed solution, which is also a concentrated supplement. In such embodiments, a supplement may be diluted more than once per each stage of supplement preparation.

In some embodiments, a metal ion, e.g., zinc, may be added to a production phase culture. The presence of zinc for the production of an anti-α4β7 antibody, such as vedolizumab, can provide reduced levels of basic isoform of the antibody (reduced relative to a control process which is the same process except for the addition of zinc). In some embodiments, zinc may be added more than once to a production phase culture. In one embodiment, zinc is added in a supplement to the starting production medium of a production phase culture. In one embodiment, zinc is added directly or in a feed solution to the production culture after the starting day, e.g., for the production phase culture. In one embodiment, zinc is added in a supplement to the starting production medium and in a supplement to the production medium after the starting day, e.g., zinc is added to a feed solution which is added to the production phase culture. In some embodiments, zinc is added in a supplement multiple times after the starting day of production phase culture. For example, zinc is added in a supplement daily, every two days, every three days, every four days, every one to three days, every two to four days or weekly. In some embodiments, the zinc that is added multiple times after the starting day of production phase culture is not added on the first, second, third, fourth, fifth or sixth day of production phase culture, but thereafter, is added daily or every two days. In one embodiment, zinc is added in a supplement to the starting medium and in a daily supplement to the production phase medium. In another embodiment, zinc is added in a supplement to the starting medium and in a daily supplement to the production phase medium beginning on day four of the production phase culture. The zinc may be supplemented until one day before harvest, two days before harvest or three days before harvest. In one embodiment, zinc is added in a supplement to the starting medium and in a daily supplement to the production phase medium beginning on day four of the production phase culture until one day before harvest.

In certain embodiments, zinc is included in a method of producing a composition having about 16% or less basic isoform (as determined by CEX) of a humanized anti-α4β7 antibody, where the humanized antibody, e.g., vedolizumab, is produced in a mammalian host cell, e.g., GS-CHO cells, in a production medium comprising zinc. In certain embodiments, adding a supplement feed containing zinc to a production medium provides a composition comprising about 14% or less basic isoform of the humanized anti-α4β7 antibody. In certain embodiments, including zinc in a supplement for a production medium provides a composition comprising about 13% or less basic isoform of the humanized anti-α4β7 antibody. In certain embodiments, including zinc in a supplement for a production medium provides a composition comprising about 12% or less basic isoform of the humanized anti-α4β7 antibody. In certain embodiments, including zinc in a supplement for a production medium provides a composition comprising about 11% or less basic isoform of the humanized anti-α4β7 antibody. In some embodiments, the level of basic isoforms can be measured at day 14 of the cell culture, i.e., 14 days after inoculation of the cell culture. In other embodiments, the level of basic isoforms can be measured at day 15 of the cell culture.

In certain embodiments, zinc is included in a method of producing a composition having about 70% or more major isoform (as determined by CEX) of a humanized anti-α4β7 antibody, where the humanized antibody, e.g., vedolizumab, is produced in a mammalian host cell, e.g., GS-CHO cells, in a production medium comprising zinc. In certain embodiments, adding a supplement feed containing zinc to a production medium provides a composition comprising about 71% or more major isoform of the humanized anti-α4β7 antibody. In certain embodiments, adding a supplement feed containing zinc to a production medium provides a composition comprising about 72% or more major isoform of the humanized anti-α4β7 antibody. In certain embodiments, adding a supplement feed containing zinc to a production medium provides a composition comprising about 73% or more major isoform of the humanized anti-α4β7 antibody. In certain embodiments, adding a supplement feed containing zinc to a production medium provides a composition comprising about 74% or more major isoform of the humanized anti-α4β7 antibody. In some embodiments, the level of major isoforms can be measured at day 14 of the cell culture. In other embodiments, the level of major isoforms can be measured at day 15 of the cell culture.

In other embodiments, zinc supplementation can be used to limit the level of HMW contaminants in a preparation comprising the anti-α4β7 antibody. In some embodiments, addition of zinc to the culture medium at a concentration of about 10-200 μM, about 50-150 μM, or about 100-130 μM can reduce the level of level of HMW aggregates to <5%, <4%, <3%, <2.5%, <2%, <1.5%, or <1% (as determined by SEC).

Zinc can be added directly to the production medium or added in a feed supplement to the production medium.

A final concentration by which the zinc ion may supplement the medium, e.g., the production phase medium, is 10 to 200 μM, 10 to 100 μM, 15 to 90 μM, 20 to 80 μM, 10 to 80 μM, 10 to 70 μM, about 14 to 55 μM, about 10 to 60 μM, about 10 to 30 μM, about 10 to 20 μM, about 14 μM, about 50 μM, about 55 μM, about 57 μM, or about 15 μM. As described above, the zinc ion may be added multiple times. In one embodiment, zinc is added to production medium of a production phase culture, such that the production medium has a concentration of zinc of about 2 to 60 μM, 5 to 57 μM, 5 to 50 μM, 5 to 40 μM, 8 to 30 μM, 10 to 20 μM, 12 to 15 μM or about 14 μM. In one embodiment, the cumulative concentration of zinc in the production medium is about 15.5 μM, accounted for by the supplementation by the time of harvest, wherein each supplement adds about 1 to about 4 μM zinc to the medium. In one embodiment, zinc is added to production medium of a production phase culture, such that the production medium has a concentration of zinc of about 50-150 μM, 75-150 μM, 100-150 μM, 80-130 μM, or 100-130 μM. In some embodiments, zinc is added to production medium of a production phase culture, such that the production medium has a concentration of zinc of about 10 μM, 20 μM, 30 μM, 40 μM, 50 μM, 60 μM, 70 μM, 80 μM, 90 μM, 100 μM, 110 μM, 120 μM, 130 μM, 140 μM, 150 μM, 160 μM, 170 μM, 180 μM, 190 μM, or 200 μM. In certain instances, too much zinc in the starting culture may decrease viability of cells and/or lower the titer of antibody.

In some embodiments, zinc is added to the culture medium (e.g., production phase culture medium) for a period of 10-16 days, e.g., 10-17 days, 10-15 days, or 12-14 days. In some embodiments, a zinc supplement can be added to the cell culture incrementally, for example, as part of a feed solution. For example, zinc can be added to the culture medium on day 0, day 1, day 2, day 3, day 4, day 5, day 6, day 7, day 8, day 9, day 10, day 11, day 12, day 13, or day 14. In some embodiments, zinc can be added daily or every other day. In some embodiments, the addition of zinc daily or every other day can begin when the cells reach production phase. Accordingly, in some embodiments, zinc can be added to the production medium daily or every other day beginning on day 4, day 5, or day 6 of the cell culture. In one embodiment, zinc can be added daily from about day 4 to about day 10. In one embodiment, zinc can be added daily from about day 4 to about day 14. In one embodiment, zinc is added as a supplement after the starting day of the production phase in order to supplement the production medium from a feed at a concentration of about 10 to 80 μM, or to a concentration of about 50 to 150 μM. In one embodiment, zinc is added to supplement the starting medium of a production phase culture such that the production medium has a zinc concentration of about 50 to 150 μM, 2 to 60 μM, 5 to 57 μM, 5 to 50 μM, 5 to 40 μM, 8 to 30 μM, 10 to 20 μM, 12 to 15 μM or about 14 μM and also is added multiple times to supplement the production medium after the starting day by 0.1 to 10 μM, 0.5 to 5 μM, 0.75 to 4 μM, 0.9 to 3 μM, 1.0 to 2.7 μM, or by about 1.4 μM or 1.9 μM with each addition to the production phase culture (e.g., beginning on a day between day two to day ten, between day two to day eight, between day two to day six, between day three to day six, or on day four of the culture). In another embodiment, zinc is added to supplement the starting medium of a production culture such that the production medium has a zinc concentration of about 2 to 50 μM, 5 to 40 μM, 8 to 30 μM, 10 to 20 μM, 12 to 15 μM or about 14 μM and also is added daily, beginning on day four of the production phase culture to supplement the production phase medium by 0.1 to 10 μM, 0.5 to 5 μM, 0.75 to 4 μM, 0.9 to 3 μM, 1.0 to 2.7 μM or by about 1.4 μM or 1.9 μM with each addition to the production phase culture. In some embodiments, the feed solution is added to the production medium such that the production medium has a total zinc concentration of about 10 to 20 μM (e.g., 15 to 17 μM) of zinc is added to the production medium. In some embodiments, the zinc supplements described herein are added to a CHO cell culture medium, for example, a culture medium provided in International Patent Publication No. WO98/08934A1, the entire contents of which are incorporated herein by reference. In some embodiments, the zinc supplements described herein are added to CD-CHO medium. In some embodiments, the zinc supplements described herein are added to CD-CHO AGT (Catalog # 12490-001 (Invitrogen, Carlsbad, Calif., USA).

In one embodiment, zinc is added to supplement a feed solution such that the feed solution has a concentration of about 90 to 120 μM, about 95 to 120 μM, about 100 to 120 μM, about 105 to 120 μM, about 110 to 120 μM, or about 117 μM. Such a feed supplement can then be added to the production medium.

In some embodiments, provided herein is a cell culture comprising a host cell (or a population of host cells) which expresses an anti-α4β7 antibody, or antigen binding portion thereof, and a production medium comprising or supplemented with zinc. In other embodiments, provided herein is a cell culture obtainable by culturing a host cell which expresses an anti-α4β7 antibody, or antigen binding portion thereof, in a production medium comprising or supplemented with zinc.

The foregoing cell culture can incorporate any of the embodiments described herein. For example, in some embodiments, the host cell is a CHO cell, for example, a GS-CHO cell, or a DHFR⁻ CHO cell. In some embodiments, the host cell expresses an antibody, or antigen binding portion thereof that comprises a heavy chain variable region of SEQ ID NO:1, and a light chain variable region of SEQ ID NO:5. In some embodiments, the host cell expresses an antibody, or antigen binding portion thereof that comprises a heavy chain variable region comprising a CDR1 described in SEQ ID NO:2, a CDR2 described in SEQ ID NO:3, and a CDR3 described in SEQ ID NO:4, and a light chain variable region comprising a CDR1 described in SEQ ID NO:6, a CDR2 described in SEQ ID NO:7 and CDR3 described in SEQ ID NO:8. In some embodiments, the host cell expresses vedolizumab, or an antigen binding portion thereof. In some embodiments, the host cell comprises a nucleic acid set forth in SEQ ID NO:9 (encoding the light chain variable region of an anti-α4β7 antibody), and a nucleic acid set forth in SEQ ID NO:10 (encoding the heavy chain variable region of an anti-α4β7 antibody). In some embodiments, the host cell comprises a nucleic acid set forth in SEQ ID NO:11 (encoding the light chain of vedolizumab) and a nucleic acid set forth as SEQ ID NO:12 (encoding the heavy chain of vedolizumab).

In some embodiments, the cell culture contains zinc at a concentration of about 10 to 100 μM, 10 to 100 μM, about 15 to 90 μM, about 20 to 80 μM, about 10 to 80 μM, about 10 to 70 μM, about 14 to 55 μM, about 10 to 60 μM, about 10 to 30 μM, about 10 to 20 μM, about 14 μM, about 50 μM, about 55 μM, about 57 μM, or about 15 μM. In some embodiments, the cell culture contains zinc at a concentration of about 2 to 60 μM, 5 to 57 μM, 5 to 50 μM, 5 to 40 μM, 8 to 30 μM, 10 to 20 μM, 12 to 15 μM or about 14 μM. In some embodiments, the cell culture contains zinc at a concentration of zinc of about 5 to 45 μM , 50-150 μM, 75-150 μM, 100-150 μM, 80-130 μM, or 100-120 μM. In some embodiments, the cell culture contains zinc at a concentration of about 1-10 μM, 10-30 μM, 30-50 μM, 50-70 μM, or 70-90 μM. In some embodiments, the cell culture contains zinc at a concentration of about 1-30 μM, 10-40 μM, 20-50 μM, 30-60 μM, 40-70 μM, or 60-90 μN. In some embodiments, the cell culture contains zinc at a concentration of about 1-50 μM, 20-60 μM, 30-70 μM, 40-80 μM, or 50-100 μM. In some embodiments, the cell culture contains zinc at a concentration of about 10 μM, 20 μM, 30 μM, 40 μM, 50 μM, 60 μM, 70 μM, 80 μM, 90 μM, 100 μM, 110 μM, 120 μM, 130 μM, 140 μM, 150 μM, 160 μM, 170 μM, 180 μM, 190 μM, or 200 μM.

In some embodiments, provided herein is a cell culture obtainable by culturing a GS-CHO host cell which expresses an anti-α4β7 antibody, or antigen binding portion thereof, in a production medium comprising or supplemented with zinc at a concentration of 50 to 150 μM, 100 to 120 μM, or 100 to 120 μM. In some embodiments, the antibody is vedolizumab, or an antigen binding portion thereof.

In some embodiments, the cells of the cell culture express an anti-α4β7 antibody, or antigen binding portion thereof having a reduced level of basic isoforms (as determined by CEX), relative to an equivalent cell culture comprising medium lacking zinc, or medium that is not supplemented with zinc. In some embodiments, the expressed antibody comprises about 16% or less basic isoforms. In some embodiments, the expressed antibody comprises about 15% or less basic isoforms. In some embodiments, the expressed antibody comprises about 14% or less basic isoforms. In some embodiments, the expressed antibody comprises about 13% or less basic isoforms. In some embodiments, the expressed antibody comprises about 12% or less basic isoforms. In some embodiments, the expressed antibody comprises about 11% or less basic isoforms.

In some embodiments, provided herein is a method of producing a monoclonal antibody, comprising (i) cultivating a cell culture provided herein comprising a host cell which expresses an anti-α4β7 antibody, or antigen binding portion thereof, and a production medium comprising or supplemented with zinc for a period of time sufficient for the host cell to express the anti-α4β7 antibody, or antigen binding portion thereof, and (ii) recovering the anti-α4β7 antibody, or antigen binding portion thereof, from the cell culture. In some embodiments, a population of anti-α4β7 antibodies, or antigen binding portions thereof, recovered from the cell culture comprises a reduced level of basic isoforms and/or an increased level of major isoform (as determined by CEX), relative to a population of anti-α4β7 antibodies, or antigen binding portions thereof, recovered from an equivalent cell culture comprising medium lacking zinc, or medium that is not supplemented with zinc. In some embodiments, a population of anti-α4β7 antibodies, or antigen binding portions thereof, recovered from the cell culture comprises a reduced level of aggregates and/or an increased level of monomers (as determined by SEC), relative to a population of anti-α4β7 antibodies, or antigen binding portions thereof, recovered from an equivalent cell culture comprising medium lacking zinc, or medium that is not supplemented with zinc. In some embodiments, the cell culture is cultivated for 5-20 days. In some embodiments, the cell culture is cultivated for 10-16 days. In some embodiments, the cell culture is cultivated for 13-15 days. In some embodiments, the cell culture is cultivated for 5, 6, 7, 8, 9, 10, 11, 12, 13, 14, 15, 16, 17, 18, 19 or 20 days. Also provided herein is an anti-α4β7 antibody that is obtained by, or obtainable by, the foregoing methods.

In each embodiment described herein, the cell culture medium can, in some embodiments, be further supplemented with a sugar, a nucleoside, and/or a metal cofactor.

For example, the cell culture medium can be further supplemented with uridine, manganese, and zinc. Additionally or alternatively, the cell culture medium can be further supplemented with lysine and/or arginine.

B. Supplementation with a Sugar, a Nucleoside, and/or a Metal Cofactor

In some embodiments, provided herein are methods and compositions for the production of a humanized anti-α4β7 antibody, e.g., vedolizumab, or an antigen binding portion thereof, in a CHO cell culture supplemented during the production phase with a sugar, a nucleoside, and/or a metal cofactor.

In some embodiments, a medium supplement for controlling the glycosylation profile of an anti-α4β7 antibody in a CHO cell culture comprises a sugar. For example, a sugar in the supplement may be glucose, fucose or galactose.

In some embodiments, a medium supplement for controlling the glycosylation profile of an anti-α4β7 antibody in a CHO cell culture comprises a nucleoside. For example, a nucleoside in the supplement may be adenosine, uridine, cytidine, guanosine, thymidine, and/or inosine.

In some embodiments, a medium supplement for controlling the glycosylation profile of an anti-α4β7 antibody in a CHO cell culture comprises a metal cofactor. For example, a metal cofactor in the supplement may be magnesium, manganese, iron or copper.

In some embodiments, a medium supplement for controlling the glycosylation profile of an anti-α4β7 antibody in a CHO cell culture comprises a sugar and a nucleoside. In some embodiments, a medium supplement for controlling the glycosylation profile of an anti-α4β7 antibody in a CHO cell culture comprises a sugar and a metal cofactor. In some embodiments, a medium supplement for controlling the glycosylation profile of an anti-α4β7 antibody in a CHO cell culture comprises a sugar, a nucleoside and a metal cofactor.

In some embodiments, provided herein are methods and compositions for the production of a humanized anti-α4β7 antibody, e.g., vedolizumab, or an antigen binding portion thereof, in a CHO cell culture supplemented during the production phase with galactose, uridine and manganese. In some embodiments, the supplemented components are in the same medium supplement. In some embodiments, the supplemented components are in different medium supplements. In some embodiments, different medium supplements are combined prior to adding to the cell culture. In some embodiments, the supplemented components for controlling the glycosylation profile of an anti-α4β7 antibody are added multiple times. For example, they may be added after the starting day of production phase culture or not added on the first, second, third, fourth, fifth or sixth day of production phase culture, but thereafter, are added daily or every two days. In one embodiment, components for controlling the glycosylation profile are added in a daily supplement to the production phase medium. In another embodiment, components for controlling the glycosylation profile are added in a daily supplement to the production phase medium beginning on day four of the production phase culture.

In some embodiments, a medium supplement for controlling glycosylation is provided to a CHO cell culture for producing an anti-α4β7 antibody during the expansion phase; in other embodiments, it is added to the production phase. In some embodiments, a medium supplement for controlling glycosylation is provided in a concentrate at 20 to 400 times, 25 to 300 times, 30 to 250 times, 40 to 120 times, about 50 times, about 60 times, about 100 times or about 200 times its final concentration in the medium. In some embodiments the amounts supplementing the medium ignores consumption by the cells, which may metabolize some of the supplemented components to other chemical forms.

In one embodiment, a metal cofactor, such as manganese, component is provided to cell culture in a medium supplement with a metal ion, such as zinc. In some embodiments, a metal cofactor, e.g., manganese, concentrate may be 10,000 to 50,000 times its final concentration in the medium, 20,000 to 40,000 times its final concentration in the medium, or about 30,000 times its final concentration in the medium.

In some embodiments, manganese may be present in the medium, or may be added to supplement the medium, e.g., the production phase medium, at a concentration of 0.1 to 100 μM, 0.5 to 50 μM, 1.0 to 25 μM, 2.0 to 15 μM, 3 to 10 μM, 1 to 50 μM, 1 to 100 μM, 20 to 50 μM, 30 to 60 μM, 40 to 70 μM, 50 to 80 μM, 70 to 100 μM, 20 to 70 μM, 30 to 80 μM, 40 to 90 μM, or 50 to 100 μM. In one embodiment, the concentration of manganese in the production phase medium is about 5.15 μM. Thus, the production phase medium can be supplemented according to a schedule to achieve an average concentration of about 5.15 μM manganese. In some embodiments, manganese may be present in the medium, or may be added to supplement the medium e.g., the production phase medium, at a concentration of about 1 μM, about 5 μM, about 10 μM, about 20 μM, about 30 μM, about 40 μM, about 50 μM, about 60 μM, about 70 μM, about 80 μM, about 90 μM, or about 100 μM.

In one embodiment, manganese is added as a supplement multiple times after the starting day to supplement the production phase culture medium by 0.1 to 10 μM, 0.2 to 1.5 μM, 0.2 to 5 μM, 0.25 to 2 μM, 0.3 to 1.2 μM, 0.3 to 0.8 μM or by about 0.5 μM or 0.56 μM with each addition. In one embodiment, manganese is added as a supplement multiple times after the starting day to supplement the production phase culture medium by about 0.2 to 1.5 μM with each addition. In one embodiment, manganese is added as a supplement multiple times after the starting day to supplement the production phase culture medium by about 0.31 to 1.2 μM with each addition. In some embodiments, a manganese supplement is added daily or every two days beginning on day four of the production phase culture. In some embodiments, the supplement is not added on the day of harvest.

In one embodiment, manganese is added as a supplement to a feed medium such that the concentration of manganese in the feed medium is at a final concentration of 0.02 mM to 0.2 mM, 0.03 mM to 0.15 mM, 0.03 mM to 0.10 mM, 0.03 mM to 0.05 mM, 0.03 mM to 0.04 mM, about 0.03 mM, about 0.04 mM, about 0.05 mM, about 0.06 mM, about 0.07 mM, about 0.08 mM, about 0.1 mM, or about 0.14 mM. In one embodiment, manganese is added as a supplement to a feed medium such that the concentration of manganese in the feed medium is at a final concentration of 0.1 to 100 μM. In one embodiment, manganese is added as a supplement to a feed medium such that the concentration of manganese in the feed medium is at a final concentration of about 39 μM. In one embodiment, the feed medium supplemented with manganese is added to the production medium (e.g., multiple times, e.g., every day or every two days) after the starting day of the production phase culture (e.g., beginning on a day between day two to day ten, between day two to day eight, between day two to day six, between day three today six, or day four of the production phase culture). In certain embodiments, the feed medium supplemented with manganese is added to the production medium beginning on day four of the production phase culture.

Uridine may be added for more than one reason. It may be added in a nutrient supplement with other nucleosides to support cell growth. Uridine also may be added as a supplement for controlling the glycosylation profile of an anti-α4β7 antibody. In some embodiments, uridine may be present in the medium, or may be added to supplement the medium, e.g., the production phase medium, at a concentration of about 0.1 to 20 mM, about 0.9 to 3.0 mM, about 1 to 20 mM, about 0.5 to 12 mM, about 1 to 8 mM, about 1.5 to 4 mM, about 0.1 to 1.5 mM, about 1 to 5 mM, about 1 to 7 mM, about 1 to 6 mM, about 1 to 5 mM, about 1 to 4 mM, about 2 to 4 mM, about 2 to 5 mM, about 2 to 3 mM, about 1 mM to 10 mM, about 10 mM to 15 mM, about 10 mM to 20 mM, about 10 mM to 30 mM, about 1 mM to 40 mM, about 1 mM to 50 mM, or about 10 mM to 30 mM. In some embodiments, uridine may be present in the medium, or may be added to supplement the medium, e.g., the production phase medium, at a concentration of about 0.9 mM, 1.0 mM, about 2 mM, about 1.5 mM, about 2.0 mM, about 2.7 mM, about 2.5 mM, about 2.7 mM, about 2.8 mM, about 5 mM, about 6 mM, about 7 mM, about 8 mM, about 9 mM, about 10 mM, about 11 mM, about 12 mM, about 13 mM, about 14 mM, about 15 mM, about 16 mM, about 17 mM, about 18 mM, about 19 mM, or about 20 mM. In some embodiments, the amounts described in the production medium account for amounts provided in the base medium or supplements and do not account for consumption, amounts metabolized or amounts produced by the cells. In some embodiments, the amounts described in the production medium are cumulative amounts as the sum of all additions by the day of harvest. In one embodiment, uridine may supplement the production phase medium by 0.1 to 20 mM, 0.5 to 12 mM, 1 to 8 mM, 1.5 to 5 mM, 1.6 to 4.8 mM or about 2.4 mM.

In one embodiment, uridine is added as a supplement multiple times after the starting day to supplement the production phase culture medium by 25 to 1000 μM, 75 to 750 μM, 55 to 620 μM, 100 to 600 μM, 150 to 450 μM, 100 to 600 μM, 170 to 630 μM, or by about 250 μM or about 300 μM with each addition. In some embodiments, these uridine supplements are added daily or every two days beginning on day four of the production phase culture and further, the uridine supplements may not be added on the day of harvest. In one embodiment, a nucleoside, e.g., uridine, containing supplement is 10 to 500 times its final concentration in the medium, 20 to 400 times, 25 to 300 times, 40 to 250 times, about 50 times, about 60 times, about 100 times or about 200 times its final concentration in the medium.

In one embodiment, uridine is added as a supplement to a feed medium such that the concentration of uridine in the feed medium is at a final concentration of about 1 to 40 mM, 15 to 25 mM, 15 to 100 mM, 20 to 90 mM, 15 to 70 mM, 15 to 50 mM, 15 to 30 mM, about 18 mM, about 19 mM, about 19.3 mM, about 20 mM, about 33 mM, about 50 mM, or about 66 mM. In one embodiment, the feed medium supplemented with uridine is added to the production medium (e.g., multiple times, e.g., every day or every two days) after the starting day of the production phase culture (e.g., beginning on a day between day two to day ten, between day two to day eight, between day two to day six, between day three to day six, or day four of the production phase culture). In certain embodiments, the feed medium supplemented with uridine is added to the production medium beginning on day four of the production phase culture. In one embodiment, a sugar, e.g., galactose, containing supplement is 10 to 500 times its final concentration in the medium, 20 to 400 times, 25 to 300 times, 30 to 250 times, 40 to 120 times, about 50 times, about 60 times, about 100 times or about 200 times its final concentration in the medium for controlling the glycosylation profile of an anti-α4β7 antibody. In some embodiments, galactose may be present in the medium, or may be added to supplement the medium, e.g., the production phase medium, at a concentration of 0.1 to 100 mM, 1 to 75 mM, 2.5 to 50 mM, 3 to 20 mM, 5 to 35 mM, about 8 to 25 mM, 0.1 to 10 mM, 0.1 to 20 mM, 0.1 to 30 mM, 1 to 10 mM, 1 to 20 mM, 1 to 30 mM, 1 to 40 mM, 1 to 50 mM, 1 to 60 mM, 1 to 70 mM, 1 to 80 mM, 1 to 90 mM, 1 to 100 mM, 20 to 40 mM, 40 to 60 mM, 60 to 80 mM, 80 to 100 mM, 20 to 50 mM, 30 to 60 mM, 40 to 70 mM, 50 to 80 mM, 70 to 100 mM, 20 to 70 mM, 30 to 80 mM, 40 to 90 mM, 50 to 100 mM, or 50 to 150 mM. In some embodiments, galactose may be present in the medium, or may be added to supplement the medium e.g., the production phase medium, at a concentration of about 0.1 mM, about 0.2 mM, about 0.3 mM, about 0.4 mM, about 0.5 mM, about 0.6 mM, about 0.7 mM, about 0.8 mM, about 0.9 mM, about 1 mM, about 2 mM-about 3 mM, about 5 mM, about 6 mM, about 7 mM, about 8 mM, about 9 mM, about 10 μM, about 12.5 mM, about 12 mM, about 12.8 mM, about 13 mM, about 15 mM, about 20 mM, about 30 mM, about 40 mM, about 50 mM, about 60 mM, about 70 mM, about 80 mM, about 90 mM, or about 100 mM. In one embodiment, galactose is added as a supplement multiple times after the starting day to supplement the production phase culture medium by 0.1 to 10 mM, 0.2 to 7.5 mM, 0.5 to 5 mM, 0.4 to 2.8 mM, 0.5 to 3.5 mM, 0.7 to 2.9 mM, 0.75 to 2.5 mM or by about 1.2 mM or 1.4 mM with each addition. In some embodiments, these galactose supplements are added daily or every two days beginning on day four of the production phase culture and further, may not be added on the day of harvest.

In one embodiment, galactose is added as a supplement to a feed medium such that the concentration of galactose in the feed medium is at a final concentration of 50 to 150 mM, 85 mM to 500 mM, 90 mM to 400 mM, 90 mM to 300 mM, 90 mM to 200 mM, 90 mM to 100 mM, about 95 mM, about 96 mM, about 97, about 100 mM, about 165 mM, about 250 mM, or about 330 mM. In one embodiment, the feed medium supplemented with galactose is added to the production medium (e.g., multiple times, e.g., every day or every two days) after the starting day of the production phase culture (e.g., beginning on a day between day two to day ten, between day two to day eight, between day two to day six, between day three to day six, or day four of the production phase culture). In certain embodiments, the feed medium supplemented with galactose is added to the production medium beginning on day four of the production phase culture.

In some embodiments, for controlling the glycosylation profile of an anti-α4β7 antibody, a production phase medium is supplemented with uridine, manganese, and galactose (UMG). In some embodiments, a UMG supplement can be added to the cell culture incrementally, for example, as part of a feed solution. For example, a feed solution containing a UMG supplement can be added daily or every two days.

In some embodiments, the supplement provides 0.1-0.7 mM uridine, 0.2-1.5 μM manganese, and 0.5-3.5 mM galactose to the production phase medium. In some embodiments, UMG can be added to the culture medium on day 0, day 1, day 2, day 3, day 4, day 5, day 6, day 7, day 8, day 9, day 10, day 11, day 12, day 13, or day 14. In some embodiments, UMG can be added daily or every other day. In some embodiments, the addition of UMG daily or every other day can begin when the cells reach production phase. Accordingly, in some embodiments, UMG can be added to the production medium daily or every other day beginning on day 4, day 5, or day 6 of the cell culture. In one embodiment, UMG can be added daily from about day 4 to about day 10. In one embodiment, UMG can be added daily from about day 4 to about day 14. In some embodiments, the production phase medium is supplemented with manganese by 1.0 to 25 μM, 2.0 to 15 μM, 3 to 10 μM or about 5 μM, e.g. by average daily additions of about 0.2-1.5 μM or 0.3 to 1.2 μM; with uridine by 1 to 8 mM, 1.5 to 5 mM, 1.6 to 4.8 mM or about 2.4 mM or 2.7 mM, e.g., by average daily additions of about 100-700 μM; and with galactose by 0.5 to 3.5 mM, 0.7 to 2.9 mM, 2.5 to 50 mM, 5 to 35 mM, about 8 to 25 mM or about 12 mM or 12.6 mM, e.g., by average daily additions of about 0.5-3.5 mM. In certain embodiments, the average daily additions begin on day four of the production phase culture. In some embodiments, the UMG supplements described herein are added to a CHO cell culture medium, for example, a culture medium provided in International Patent Publication No. WO98/08934A1, the entire contents of which are incorporated herein by reference. In some embodiments, the UMG supplements described herein are added to CD-CHO medium. In some embodiments, the UMG supplements described herein are added to CD-CHO AGT (Catalog #12490-001 (Invitrogen, Carlsbad, Calif., USA).

In one embodiment, UMG is added to the production medium such that the cumulative concentration of UMG added from supplementation to harvest is about 1-7 mM uridine, about 2-15 μM manganese, and f about 3-20 mM galactose. In some embodiments, the cumulative concentration of zinc added to the production medium from supplementation to harvest is about 5-45 μM.

In one embodiment, uridine, manganese, and galactose (UMG) is added as a supplement to a feed medium such that the concentration of uridine in the feed medium is at a final concentration of 1 to 40 mM, 15 to 100 mM, 15 to 90 mM, 15 to 70 mM, 15 to 50 mM, 15 to 30 mM, about 18 mM, about 19 mM, about 20 mM, about 21 mM, about 33 mM, about 50 mM, or about 66 mM uridine; the concentration of manganese in the feed medium is at a final concentration of about 0.0001 to 0.1 mM, 0.02 mM to 0.2 mM, 0.03 mM to 0.15 mM, 0.03 mM to 0.10 mM, 0.03 mM to 0.05 mM, 0.03 mM to 0.04 mM, about 0.03 mM, about 0.04 mM, about 0.05 mM, about 0.06 mM, about 0.07 mM, about 0.08 mM, about 0.1 mM, or about 0.14 mM manganese; and the concentration of galactose in the feed medium is at a final concentration of 85 mM to 500 mM, 90 mM to 400 mM, 90 mM to 300 mM, 90 mM to 200 mM, 50 mM to 150 mM, 90 mM to 100 mM, about 95 mM, about 96 mM, about 97 mM, about 100 mM, about 165 mM, about 250 mM, or about 330 mM galactose. In one embodiment, the feed medium supplemented with uridine, manganese, and galactose is added to the production medium (e.g., multiple times, e.g., every day or every two days) after the starting day of the production phase culture (e.g., beginning on a day between day two to day ten, between day two to day eight, between day two to day six, between day three to day six, or day four of the production phase culture). In certain embodiments, the feed medium supplemented with uridine, manganese, and galactose is added to the production medium, e.g., daily, beginning on day four of the production phase culture.

In certain embodiments, a combined supplement containing uridine, manganese and galactose (UMG) is added to the production medium (or to a feed solution that is subsequently added to a production medium) for producing a composition comprising an anti-α4β7 antibody, such as vedolizumab, where the composition has a reduced level of basic antibody isoform. In one embodiment, the level of basic isoform is about 16% or less (as determined by CEX). In one embodiment, the level of basic isoform is about 15% or less (as determined by CEX). In one embodiment, the level of basic isoform is about 14% or less (as determined by CEX). In one embodiment, the level of basic isoform is about 13% or less (as determined by CEX). In one embodiment, the level of basic isoform is about 12% or less (as determined by CEX).

The addition of UMG to production medium can also impact the level of acidic species and/or main species of antibody in the composition of anti-α4β7 antibodies, such as vedolizumab, produced by mammalian cells.

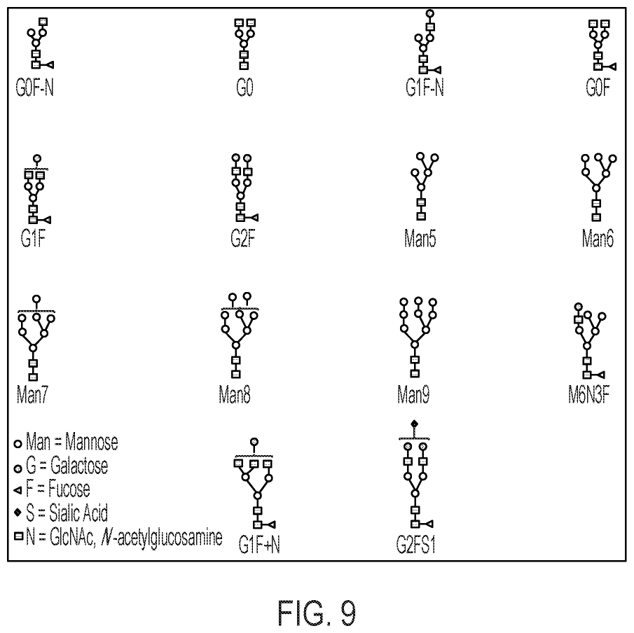

The addition of UMG to production medium can also impact the level of G0F, G1F, and/or G2F glycoforms of antibody in the composition of anti-α4β7 antibodies, such as vedolizumab, produced by mammalian cells. A depiction of the structure of N-glycans that can be present in a population of an anti-α4β7 antibody, such as vedolizumab, is provided as FIG. 9.

In certain embodiments, a combined supplement containing uridine, manganese and galactose (UMG) is added to the production medium (or to a feed solution that is subsequently added to a production medium) for producing a composition comprising an anti-α4β7 antibody, such as vedolizumab, where the composition has a decreased level of G0F glycoform. In one embodiment, the level of G0F glycoform is about 70% or less (as determined by Hydrophilic Interaction Chromatography (HILIC)). In one embodiment, the level of G0F glycoform is about 69% or less (as determined by HILIC). In one embodiment, the level of G0F glycoform is about 68% or less (as determined by HILIC). In one embodiment, the level of G0F glycoform is about 67% or less (as determined by HILIC). In one embodiment, the level of G0F glycoform is about 66% or less (as determined by HILIC). In one embodiment, the level of G0F glycoform is about 65% or less (as determined by HILIC). In one embodiment, the level of G0F glycoform is about 64% or less (as determined by HILIC). In one embodiment, the level of G0F glycoform is about 63% or less (as determined by HILIC). In one embodiment, the level of G0F glycoform is about 62% or less (as determined by HILIC). In one embodiment, the level of G0F glycoform is about 61% or less (as determined by HILIC). In one embodiment, the level of G0F glycoform is about 60% or less (as determined by HILIC). In one embodiment, the level of G0F glycoform is about 59% or less (as determined by HILIC). In one embodiment, the level of G0F glycoform is about 58% or less (as determined by HILIC). In one embodiment, the level of G0F glycoform is about 57% or less (as determined by HILIC). In one embodiment, the level of G0F glycoform is about 56% or less (as determined by HILIC). In one embodiment, the level of G0F glycoform is about 55% or less (as determined by HILIC). In one embodiment, the level of G0F glycoform is about 40-75%. In one embodiment, the level of G0F glycoform is about 45-65% (as determined by HILIC). In one embodiment, the level of G0F glycoform is about 50-60% (as determined by HILIC).

In certain embodiments, a combined supplement containing uridine, manganese and galactose (UMG) is added to the production medium (or to a feed solution that is subsequently added to a production medium) for producing a composition comprising an anti-α4β7 antibody, such as vedolizumab, where the composition has a decreased amount of G0F glycoform in comparison to a control mammalian host cell expressing the anti-α4β7 antibody that is cultured in absence of the supplement. In one embodiment, the composition comprises at least about a 20%-40% (e.g., 20%-40%, 20%-30%, 20%-25%) decrease in the G0F glycoform of the humanized anti-α4β7 antibody in comparison to a control mammalian host cell expressing the humanized anti-α4β7 antibody that is cultured in the absence of the supplement. In one embodiment, the composition comprises at least about a 20% decrease in the G0F glycoform of the humanized anti-α4β7 antibody in comparison to a control mammalian host cell expressing the humanized anti-α4β7 antibody that is cultured in the absence of the supplement. In one embodiment, the composition comprises at least about a 25% decrease in the G0F glycoform of the humanized anti-α4β7 antibody in comparison to a control mammalian host cell expressing the humanized anti-α4β7 antibody that is cultured in the absence of the supplement. Comparative controls are performed under under substantially similar conditions other than the parameter specified as being different, e.g., the absence of a supplement.

In certain embodiments, a combined supplement containing uridine, manganese and galactose (UMG) is added to the production medium (or to a feed solution that is subsequently added to a production medium) for producing a composition comprising an anti-α4β7 antibody, such as vedolizumab, where the composition has an increased level of G1F glycoform. In one embodiment, the level of G1F glycoform is about 20% or more (as determined by HILIC). In one embodiment, the level of G1F glycoform is about 21% or more (as determined by HILIC). In one embodiment, the level of G1F glycoform is about 22% or more (as determined by HILIC). In one embodiment, the level of G1F glycoform is about 23% or more (as determined by HILIC). In one embodiment, the level of G1F glycoform is about 24% or more (as determined by HILIC). In one embodiment, the level of G1F glycoform is about 25% or more (as determined by HILIC). In one embodiment, the level of G1F glycoform is about 26% or more (as determined by HILIC). In one embodiment, the level of G1F glycoform is about 27% or more (as determined by HILIC). In one embodiment, the level of G1F glycoform is about 28% or more (as determined by HILIC). In one embodiment, the level of G1F glycoform is about 29% or more (as determined by HILIC). In one embodiment, the level of G1F glycoform is about 30% or more (as determined by HILIC). In one embodiment, the level of G1F glycoform is about 31% or more (as determined by HILIC). In one embodiment, the level of G1F glycoform is about 32% or more (as determined by HILIC). In one embodiment, the level of G1F glycoform is about 33% or more (as determined by HILIC). In one embodiment, the level of G1F glycoform is about 20-45%. In one embodiment, the level of G1F glycoform is about 25-45% (as determined by HILIC). In one embodiment, the level of G1F glycoform is about 30-40% (as determined by HILIC).

In certain embodiments, a combined supplement containing uridine, manganese and galactose (UMG) is added to the production medium (or to a feed solution that is subsequently added to a production medium) for producing a composition comprising an anti-α4β7 antibody, such as vedolizumab, where the composition has an increased amount of G1F glycoform in comparison to a control cell culture comprising a mammalian host cell expressing the anti-α4β7 antibody that is cultured in absence of the supplement. In one embodiment, the composition comprises at least about a 2-fold to 3.5-fold (e.g., 2- to 3.5-fold, 2- to 3.3-fold, 2- to 3-fold) increase in the G1F glycoform of the humanized anti-α4β7 antibody in comparison to a control cell culture comprising a mammalian host cell expressing the humanized anti-α4β7 antibody that is cultured in the absence of the supplement. In one embodiment, the composition comprises at least about a 2-fold increase in the G1F glycoform of the humanized anti-α4β7 antibody in comparison to a control cell culture comprising a mammalian host cell expressing the humanized anti-α4β7 antibody that is cultured in the absence of the supplement. In one embodiment, the composition comprises at least about a 3-fold increase in the G1F glycoform of the humanized anti-α4β7 antibody in comparison to a control cell culture comprising a mammalian host cell expressing the humanized anti-α4β7 antibody that is cultured in the absence of the supplement. The control cell culture is cultured under substantially the same conditions with the exception of the specified parameter, e.g., the supplement.

In certain embodiments, a combined supplement containing uridine, manganese and galactose (UMG) is added to the production medium (or to a feed solution that is subsequently added to a production medium) for producing a composition comprising an anti-04f37 antibody, such as vedolizumab, where the composition has an increased level of G2F glycoform. In one embodiment, the level of G2F glycoform is about 2% or more (as determined by HILIC). In one embodiment, the level of G2F glycoform is about 2.5% or more (as determined by HILIC). In one embodiment, the level of G2F glycoform is about 3% or more (as determined by HILIC). In one embodiment, the level of G2F glycoform is about 3.5% or more (as determined by HILIC). In one embodiment, the level of G2F glycoform is about 4% or more (as determined by HILIC). In one embodiment, the level of G2F glycoform is about 4.5% or more (as determined by HILIC). In one embodiment, the level of G2F glycoform is about 5% or more (as determined by HILIC). In one embodiment, the level of G2F glycoform is about 5.5% or more (as determined by HILIC). In one embodiment, the level of G2F glycoform is about 6% or more (as determined by HILIC). In one embodiment, the level of G2F glycoform is about 6.5% or more (as determined by HILIC). In one embodiment, the level of G2F glycoform is about 7% or more (as determined by HILIC). In one embodiment, the level of G2F glycoform is less than or equal to 10%. In one embodiment, the level of G2F glycoform is about 2-4% (as determined by HILIC). In one embodiment, the level of G2F glycoform is about 3-5% (as determined by HILIC). In one embodiment, the level of G2F glycoform is about 2-7% (as determined by HILIC).

In certain embodiments, a combined supplement containing uridine, manganese and galactose (UMG) is added to the production medium (or to a feed solution that is subsequently added to a production medium) for producing a composition comprising an anti-α4β7 antibody, such as vedolizumab, where the composition has an increased level of G2F glycoform. In one embodiment, the level of G2F glycoform is about 2% or more (as determined by HILIC). In one embodiment, the level of G2F glycoform is about 3% or more (as determined by HILIC). In one embodiment, the level of G2F glycoform is about 4% or more (as determined by HILIC).

In certain embodiments, a combined supplement containing uridine, manganese and galactose (UMG) is added to the production medium (or to a feed solution that is subsequently added to a production medium) for producing a composition comprising an anti-α4β7 antibody, such as vedolizumab, where the composition has an increased amount of G2F glycoform in comparison to a control mammalian host cell expressing the anti-α4β7 antibody that is cultured in absence of the supplement. In one embodiment, the composition comprises at least about a 2-fold to 5-fold (e.g., 2- to 5-fold, 2- to 4-fold, 3- to 4-fold) increase in the G2F glycoform of the humanized anti-α4β7 antibody in comparison to a control cell culture comprising a mammalian host cell expressing the humanized anti-α4β7 antibody that is cultured in the absence of the supplement. In one embodiment, the composition comprises at least about a 3-fold increase in the G2F glycoform of the humanized anti-α4β7 antibody in comparison to a control cell culture comprising a mammalian host cell expressing the humanized anti-α4β7 antibody that is cultured in the absence of the supplement. In one embodiment, the composition comprises at least about a 4-fold increase in the G2F glycoform of the humanized anti-α4β7 antibody in comparison to a control cell culture comprising a mammalian host cell expressing the humanized anti-α4β7 antibody that is cultured in the absence of the supplement. The control cell culture is cultured under substantially the same conditions with the exception of the specified parameter, e.g., the supplement.

In certain embodiments, a combined supplement containing uridine, manganese and galactose (UMG) is added to the production medium (or to a feed solution that is subsequently added to a production medium) for producing a composition comprising an anti-α4β7 antibody, such as vedolizumab, where the composition has an increased amount of G1F and G2F glycoforms in comparison to a control cell culture comprising a mammalian host cell expressing the anti-α4β7 antibody that is cultured in absence of the supplement. In one embodiment, the composition comprises at least about a 2-fold to 5-fold (e.g., 2- to 5-fold, 2- to 4-fold, 3- to 4-fold) increase in the G1F glycoform and at least about a 2-fold to 5-fold (e.g., 2- to 5-fold, 2- to 4-fold, 3- to 4-fold) increase in the G2F glycoform of the humanized anti-α4β7 antibody in comparison to a control cell culture comprising a mammalian host cell expressing the humanized anti-α4β7 antibody that is cultured in the absence of the supplement. In one embodiment, the composition comprises at least about a 3-fold increase in the G1F and G2F glycoforms of the humanized anti-α4β7 antibody in comparison to a control cell culture comprising a mammalian host cell expressing the humanized anti-α4β7 antibody that is cultured in the absence of the supplement. In one embodiment, the composition comprises at least about a 2-fold increase in the amount of G1F glycoform and a 4-fold increase in the G2F glycoform of the humanized anti-α4β7 antibody in comparison to a control cell culture comprising a mammalian host cell expressing the humanized anti-α4β7 antibody that is cultured in the absence of the supplement. The control cell culture is cultured under substantially the same conditions with the exception of the specified parameter, e.g., the supplement.

In certain embodiments, a combined supplement containing uridine, manganese and galactose (UMG) is added to the production medium (or to a feed solution that is subsequently added to a production medium) for producing a composition comprising an anti-α4β7 antibody, such as vedolizumab, where the composition has 88% or more, 90% or more, 91% or more, 92% or more, 93% or more, 94% or more, or 95% or more total asialo-, agalacto, core fucosylated biantennary glycan (G0F), asialo-, monogalacto, core fucosylated biantennary glycan (G1F), and/or asialo-, digalacto, core fucosylated biantennary glycan (G2F) glycosylation variants. In some embodiments, the compositions and methods described herein can produce a population of humanized anti-α4β7 antibodies having 91-96%, 92-95%, 91-92%, 91-92.5%, 91-93%, or 91-95% total asialo-, agalacto, core fucosylated biantennary glycan (G0F), asialo-, monogalacto, core fucosylated biantennary glycan (G1F), and/or asialo-, digalacto, core fucosylated biantennary glycan (G2F) glycosylation variants. In some embodiments, the compositions and methods described herein can produce a population of humanized anti-α4β7 antibodies having 92% to 98%, 92% to 97%, 92% to 96%, or 92% to 95% total asialo-, agalacto, core fucosylated biantennary glycan (G0F), asialo-, monogalacto, core fucosylated biantennary glycan (G1F), and/or asialo-, digalacto, core fucosylated biantennary glycan (G2F) glycosylation variants,

A medium supplement may be provided in water, base culture medium or in a buffer, such as ascorbate, citrate, carbonate, (4-(2-hydroxyethyl)-1-piperazineethanesulfonic acid (HEPES), histidine, glutamate, acetate, succinate, gluconate, histidine, phosphate, maleate, cacodylate, 2-[N-morpholino]ethanesulfonic acid (MES), bis(2-hydroxyethyl)iminotris[hydroxymethyl]methane (B is -Tris), N-[2-acetamido]-2-iminodiacetic acid (ADA), glycylglycine and other organic acid or zwitterionic buffers. In some embodiments, a medium supplement has a pH of 5.5 to 7.0, 6.0 to 7.5 or 5.9 to 6.1. In other embodiments, a medium supplement has a pH of 1.5 to 5.5, 1.8 to 3.0, 3.2 to 4.5 or 1.9 to 2.1. In other embodiments, a medium supplement has a pH of 7.5 to 9.0.

In some embodiments, zinc and UMG are used for supplementing the feed solution added daily beginning on day four of the production phase culture.

In certain embodiments, a metal, e.g., a zinc- or manganese-containing supplement is a buffer at a low pH, such as a citrate or acetate buffer. Citrate also may function to chelate the metal ion to limit toxicity of the metal ion supplement. In one embodiment, a metal containing supplement comprises zinc and manganese, e.g., zinc and manganese in citrate buffer at 100 to 140 mM or 115 to 125 mM. In one embodiment, a buffer for a metal containing supplement comprises 118 to 122 mM citric acid, pH 1.9 to 2.1. Accordingly, in some embodiments, provided herein is a cell culture obtainable by culturing a GS-CHO host cell which expresses an anti-α4β7 antibody, or antigen binding portion thereof, in a production medium supplemented by 50-150 μM zinc and 10-50 mM manganese solution in a 115 to 125 mM citrate buffer pH 1.9 to 2.1. In some embodiments, provided herein is a cell culture obtainable by culturing a GS-CHO host cell which expresses an anti-α4β7 antibody, or antigen binding portion thereof, in a production medium supplemented to a concentration of 10-100 μM zinc and 0.1-100 μM manganese, by the addition of zinc and manganese in a 115 to 125 mM citrate buffer feed supplement at pH 1.9 to 2.1. In some embodiments the citrate buffered metal supplement is added to the daily feed supplement, e.g., beginning at day 4 of the production culture.

In some embodiments, provided herein is a cell culture comprising a host cell (or a population of host cells) which expresses an anti-α4β7 antibody, or antigen binding portion thereof, and a production medium comprising or supplemented with a metal ion, a nucleoside, a sugar, and/or a metal cofactor. In other embodiments, provided herein is a cell culture obtainable by culturing a host cell which expresses an anti-α4β7 antibody, or antigen binding portion thereof, in a production medium comprising or supplemented with a metal ion, a nucleoside, a sugar, and/or a metal cofactor.

In some embodiments, provided herein is a cell culture comprising a host cell (or a population of host cells) which expresses an anti-α4β7 antibody, or antigen binding portion thereof, and a production medium comprising or supplemented with a sugar, a nucleoside and/or a metal cofactor. In other embodiments, provided herein is a cell culture obtainable by culturing a host cell which expresses an anti-α4β7 antibody, or antigen binding portion thereof, in a production medium comprising or supplemented with a sugar, a nucleoside and/or a metal cofactor.

In some embodiments, provided herein is a cell culture comprising a host cell (or a population of host cells) which expresses an anti-α4β7 antibody, or antigen binding portion thereof, and a production medium comprising or supplemented with uridine, manganese, and galactose (UMG). In other embodiments, provided herein is a cell culture obtainable by culturing a host cell which expresses an anti-α4β7 antibody, or antigen binding portion thereof, in a production medium comprising or supplemented with uridine, manganese, and galactose (UMG).

The foregoing cell culture can incorporate any of the embodiments described herein. For example, in some embodiments, the host cell is a CHO cell, e.g., a GS-CHO cell, or a DHFR⁻ CHO cell. In some embodiments, the host cell expresses an antibody, or antigen binding portion thereof that comprises a heavy chain variable region of SEQ ID NO:1, and a light chain variable region of SEQ ID NO:5. In some embodiments, the host cell expresses an antibody, or antigen binding portion thereof that comprises a heavy chain variable region comprising a CDR1 described in SEQ ID NO:2, a CDR2 described in SEQ ID NO:3, and a CDR3 described in SEQ ID NO:4, and a light chain variable region comprising a CDR1 described in SEQ ID NO:6, a CDR2 described in SEQ ID NO:7 and CDR3 described in SEQ ID NO:8. In some embodiments, the host cell expresses vedolizumab, or an antigen binding portion thereof. In some embodiments, the host cell comprises a nucleic acid set forth in SEQ ID NO:9 (encoding the light chain variable region of an anti-α4β7 antibody), and a nucleic acid set forth in SEQ ID NO:10 (encoding the light chain variable region of an anti-α4β7 antibody). In some embodiments, the host cell comprises a nucleic acid set forth in SEQ ID NO:11 (encoding the light chain of vedolizumab) and a nucleic acid set forth as SEQ ID NO:12 (encoding the heavy chain of vedolizumab).

In some embodiments, the cell culture contains uridine at a concentration of 0.1 to 20 mM. For example, in some embodiments, the cell culture contains 0.1 to 20 mM, 1 to 20 mM, 0.5 to 12 mM, 1 to 8 mM, 1.5 to 4 mM, 0.1 to 1.5 mM, 0.1 to 5 mM, 5 mM to 10 mM, 10 mM to 15 mM, 15 mM to 20 mM, 0.1 mM to 10 mM, 10 mM to 20 mM, 1 to 7 mM, 7 to 14 mM, or 14 to 20 mM. In other embodiments, the cell culture contains uridine at a concentration of 10-50 mM, 20-60 mM, 30-70 mM, 40-80 mM, 50-90 mM, 60-100 mM, or 0.1 to 100 mM. In some embodiments, the cell culture contains uridine at a concentration of about 10 mM, about 15 mM, about 16 mM, about 17 mM, about 18 mM, about 19 mM, about 20 mM, about 21 mM, about 22 mM, about 25 mM, about 27 mM, about 30 mM, about 33 mM, about 35 mM, about 40 mM, about 45 mM, about 50 mM, about 55 mM, about 60 mM, about 66 mM, or about 70 mM.

In some embodiments, the cell culture contains manganese at a concentration of 0.1 to 100 04. For example, in some embodiments, the cell culture contains manganese at a concentration of 0.1 to 100 μM, 0.5 to 50 μM, 1.0 to 25 μM, 2.0 to 15 μM , 3 to 10 μM, 0.1 to 10 μM, 0.1 to 20 μM, 0.1 to 30 μM, 1 to 10 μM, 1 to 20 μM, 1 to 30 μM, 1 to 40 μM, 1 to 50 μM, 1 to 60 μM, 1 to 70 μM, 1 to 80 μM, 1 to 90 μM, 1 to 100 μM, 20 to 40 μM, 40 to 60 μM, 60 to 80 μM, 80 to 100 μM, 20 to 50 μM, 30 to 60 μM, 40 to 70 μM, 50 to 80 μM, 70 to 100 μM, 20 to 70 μM, 30 to 80 μM, 40 to 90 μM, or 50 to 100 μM. In other embodiments, the cell culture contains manganese at a concentration of about 0.1 μM, about 0.2 μM, about 0.3 μM, about 0.4 μM, about 0.5 μM, about 0.6 μM, about 0.7 μM, about 0.8 μM, about 0.9 μM, about 1 μM, about 2 μM, about 3 μM, about 5 μM, about 10 μM, about 20 μM, about 30 μM, about 40 μM, about 50 μM, about 60 μM, about 70 μM, about 80 μM, about 90 μM, or about 100 μM.

In some embodiments, the cell culture contains galactose at a concentration of 0.1 to 100 mM. For example, in some embodiments, the cell culture contains galactose at a concentration of 1 to 75 mM, 2.5 to 50 mM, 5 to 35 mM, about 8 to 25 mM, 0.1 to 10 mM, 0.1 to 20 mM, 0.1 to 30 mM, 1 to 10 mM, 1 to 20 mM, 1 to 30 mM, 1 to 40 mM, 1 to 50 mM, 1 to 60 mM, 1 to 70 mM, 1 to 80 mM, 1 to 90 mM, 1 to 100 mM, 20 to 40 mM, 40 to 60 mM, 60 to 80 mM, 80 to 100 mM, 20 to 50 mM, 30 to 60 mM, 40 to 70 mM, 50 to 80 mM, 70 to 100 mM, 20 to 70 mM, 30 to 80 mM, 40 to 90 mM, or 50 to 100 mM. In some embodiments, the cell culture contains galactose at a concentration of about 0.1 mM, about 0.2 mM, about 0.3 mM, about 0.4 mM, about 0.5 mM, about 0.6 mM, about 0.7 mM, about 0.8 mM, about 0.9 mM, about 1 mM, about 2 mM,about 3 mM, about 5 mM, about 6 mM, about 7 mM, about 8 mM, about 9 mM, about 10 μM, about 12.5 mM, about 12 mM, about 15 mM, about 20 mM, about 30 mM, about 40 mM, about 50 mM, about 60 mM, about 70 mM, about 80 mM, about 90 mM, or about 100 mM.

In some embodiments, provided herein is a cell culture obtainable by culturing a host cell (or a population of host cells) which expresses an anti-α4β7 antibody, or antigen binding portion thereof, in a production medium comprising or supplemented with uridine, manganese, and galactose. For example, in some embodiments, the cell culture can comprise 0.1 to 20 mM uridine (and ranges therein), 0.1 to 100 μM manganese (and ranges therein), and 0.1 to 100 mM galactose (and ranges therein). In some embodiments, the cell culture can additionally comprise zinc, as described herein. In some embodiments, the cell culture can additionally comprise lysine and/or arginine, as described herein. In some embodiments, the cell culture can additionally comprise zinc, lysine, and arginine. In some embodiments, provided herein is a cell culture comprising a host cell (or a population of host cells) which expresses an anti-α4β7 antibody, or antigen binding portion thereof, (e.g., vedolizumab) and a production medium to which a cumulative concentration of about 1 to about 7 mM uridine, about 2 to about 15 μM manganese, about 3 to about 20 mM galactose, and/or about 0.005 to about 0.045 mM zinc has been added during the production phase, e.g., day 4 to harvest.

In some embodiments, provided herein is a cell culture obtainable by culturing a host cell (or a population of host cells) which expresses an anti-α4β7 antibody, or antigen binding portion thereof, in a production medium comprising or supplemented with uridine, manganese, galactose, and zinc. For example, in some embodiments, the cell culture can comprise 0.1 to 20 mM uridine (and ranges therein), 0.1 to 100 μM manganese (and ranges therein), 0.1 to 100 mM galactose (and ranges therein), and 10 to 100 μM zinc (and ranges therein).

In some embodiments, provided herein is a cell culture obtainable by culturing a host cell (or a population of host cells) which expresses an anti-α4β7 antibody, or antigen binding portion thereof, in a production medium comprising or supplemented with uridine, manganese, galactose, zinc, lysine and/or arginine. For example, in some embodiments, the cell culture can comprise 0.1 to 20 mM uridine (and ranges therein), 0.1 to 100 μM manganese (and ranges therein), 0.1 to 100 mM galactose (and ranges therein), 10 to 100 μM zinc (and ranges therein), 5.0 to 8.8 g/L lysine (and ranges therein) and/or 3.0 to 12.0 g/L arginine (and ranges therein).

In some embodiments, the cells of the cell culture express an anti-α4β7 antibody, or antigen binding portion thereof having a reduced level of basic isoforms (as determined by CEX), relative to an equivalent cell culture comprising medium lacking uridine, manganese, and/or galactose, or medium that is not supplemented with uridine, manganese, and/or galactose. In some embodiments, the expressed antibody comprises about 16% or less basic isoforms (as determined by CEX). In some embodiments, the expressed antibody comprises about 15% or less basic isoforms (as determined by CEX). In some embodiments, the expressed antibody comprises about 14% or less basic isoforms (as determined by CEX). In some embodiments, the expressed antibody comprises about 13% or less basic isoforms (as determined by CEX). In some embodiments, the expressed antibody comprises about 12% or less basic isoforms (as determined by CEX). In some embodiments, the expressed antibody comprises about 11% or less basic isoforms (as determined by CEX).

In some embodiments, the cells of the cell culture express an anti-α4β7 antibody, or antigen binding portion thereof having a reduced level of G0F glycoforms (as determined by HILIC), relative to an equivalent cell culture comprising medium lacking uridine, manganese, and/or galactose, or medium that is not supplemented with uridine, manganese, and/or galactose. In some embodiments, the cells of the cell culture express an anti-α4β7 antibody having a G0F content of 70% or less, 65% or less, 60% or less or 55% or less (as determined by HILIC). In some embodiments, the cells of the cell culture express an anti-α4β7 antibody having a G0F content of 85% or less, 80% or less, 75% or less, 70% or less, 69% or less, 68% or less, 67% or less, 66% or less, 65% or less, 64% or less, 63% or less, 62% or less, 61% or less, 60% or less, 59% or less, 58% or less, 57% or less, 56% or less, or 55% or less (as determined by HILIC). In some embodiments, the cells of the cell culture express an anti-α4β7 antibody having a G0F content of 45-65%. In some embodiments, the cells of the cell culture express an anti-α4β7 antibody having a G0F content of 50-60%. In some embodiments, the cells of the cell culture express an anti-α4β7 antibody having a G0F content of 45-85%. In some embodiments, the cells of the cell culture express an anti-α4β7 antibody having a G0F content of 45-82%. In some embodiments, the G0F content of an anti-α4β7 antibody produced by a cell culture comprising medium containing or supplemented with uridine, manganese, and/or galactose as described herein is reduced by at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, or at least 40%, relative to the G0F content of an equivalent anti-α4β7 antibody produced by an equivalent cell culture comprising medium lacking uridine, manganese, and/or galactose, or medium that is not supplemented with uridine, manganese, and/or galactose.

In some embodiments, the cells of the cell culture express an anti-α4β7 antibody, or antigen binding portion thereof having an increased level of G1F glycoforms (as determined by HILIC), relative to an equivalent cell culture comprising medium lacking uridine, manganese, and/or galactose, or medium that is not supplemented with uridine, manganese, and/or galactose. In some embodiments, the cells of the cell culture express an anti-α4β7 antibody having a G1F content of 10% or more, 15% or more, 20% or more, 21% or more, 22% or more, 23% or more, 24% or more, 25% or more, 26% or more, 27% or more, 28% or more, 29% or more, 30% or more, 31% or more, 32% or more, or 33% or more (as determined by HILIC). In some embodiments, the cells of the cell culture express an anti-α4β7 antibody having a G1F content of 25-45%. In some embodiments, the cells of the cell culture express an anti-α4β7 antibody having a G1F content of 30-40%. In some embodiments, the cells of the cell culture express an anti-α4β7 antibody having a G1F content of 10-45%. In some embodiments, the G1F content of an anti-α4β7 antibody produced by a cell culture comprising medium containing or supplemented with uridine, manganese, and/or galactose as described herein is increased by at least 2-fold, by at least 2.25-fold, by at least 2.5-fold, by at least 2.75-fold, by at least 3-fold, by at least 3.25-fold, or by at least 3.5-fold, relative to the G1F content of an equivalent anti-α4β7 antibody produced by an equivalent cell culture comprising medium lacking uridine, manganese, and/or galactose, or medium that is not supplemented with uridine, manganese, and/or galactose.

In some embodiments, the cells of the cell culture express an anti-α4β7 antibody, or antigen binding portion thereof having an increased level of G2F glycoforms (as determined by HILIC), relative to an equivalent cell culture comprising medium lacking uridine, manganese, and/or galactose, or medium that is not supplemented with uridine, manganese, and/or galactose. In some embodiments, the cells of the cell culture express an anti-α4β7 antibody having a G2F content of 0.5% or more, 1% or more, 1.5% or more, 2% or more, 2.5% or more, 3% or more, 3.5% or more, 4% or more, 4.5% or more, 5% or more, 5.5% or more, 6% or more, 6.5% or more, 7% or more, or 8% or more (as determined by HILIC). In some embodiments, the cells of the cell culture express an anti- α4f37 antibody having a G2F content of 2-4%. In some embodiments, the cells of the cell culture express an anti-α4β7 antibody having a G2F content of 3-5%. In some embodiments, the cells of the cell culture express an anti-α4β7 antibody having a G2F content of 2-7%. In some embodiments, the cells of the cell culture express an anti-α4β7 antibody having a G2F content of 0.5-7.5%. In some embodiments, the G2F content of an anti-α4β7 antibody produced by a cell culture comprising medium containing or supplemented with uridine, manganese, and/or galactose as described herein is increased by at least 2-fold, by at least 2.25-fold, by at least 2.5-fold, by at least 2.75-fold, by at least 3-fold, by at least 3.25-fold, by at least 3.5-fold, by at least 3,75-fold, by at least 4-fold, by at least 4.25-fold, by at least 4.5-fold, by at least 4.75-fold, or by at least 5-fold, relative to the G1F content of an equivalent anti-α4β7 antibody produced by an equivalent cell culture comprising medium lacking uridine, manganese, and/or galactose, or medium that is not supplemented with uridine, manganese, and/or galactose.

A cell culture provided herein can, in some embodiments, produce a population of humanized anti-α4β7 antibodies where the population has 88% or more, 90% or more, 91% or more, 92% or more, 93% or more, 94% or more, or 95% or more total asialo-, agalacto, core fucosylated biantennary glycan (G0F), asialo-, monogalacto, core fucosylated biantennary glycan (G1F), and/or asialo-, digalacto, core fucosylated biantennary glycan (G2F) glycosylation variants (as determined by HILIC). In some embodiments, the cell culture can produce a population of humanized anti-α4β7 antibodies having 91-96%, 92-95%, 91-92%, 91-92.5%, 91-93%, or 91-95% total asialo-, agalacto, core fucosylated biantennary glycan (G0F), asialo-, monogalacto, core fucosylated biantennary glycan (G1F), and/or asialo-, digalacto, core fucosylated biantennary glycan (G2F) glycosylation variants (as determined by HILIC). In some embodiments, the cell culture can produce a population of humanized anti-α4β7 antibodies having 92% to 98%, 92% to 97%, 92% to 96%, or 92% to 95% total asialo-, agalacto, core fucosylated biantennary glycan (G0F), asialo-, monogalacto, core fucosylated biantennary glycan (G1F), and/or asialo-, digalacto, core fucosylated biantennary glycan (G2F) glycosylation variants.

In some embodiments, provided herein is a method of producing a monoclonal antibody, comprising (i) cultivating a cell culture comprising a host cell which expresses an anti-α4β7 antibody, or antigen binding portion thereof, and a production medium comprising or supplemented with uridine, manganese, and/or galactose for a period of time sufficient for the host cell to express the anti-α4β7 antibody, or antigen binding portion thereof, and (ii) recovering the anti-α4β7 antibody, or antigen binding portion thereof, from the cell culture. In some embodiments, a population of anti-α4β7 antibodies, or antigen binding portions thereof, recovered from the cell culture comprises a reduced level of basic isoforms (as determined by CEX), relative to a population of anti-α4β7 antibodies, or antigen binding portions thereof, recovered from an equivalent cell culture comprising medium lacking uridine, manganese, and/or galactose, or medium that is not supplemented with uridine, manganese, and/or galactose. In some embodiments, a population of anti-α4β7 antibodies, or antigen binding portions thereof, recovered from the cell culture comprises a reduced level of G0F), relative to a population of anti-α4β7 antibodies, or antigen binding portions thereof, recovered from an equivalent cell culture comprising medium lacking uridine, manganese, and/or galactose, or medium that is not supplemented with uridine, manganese, and/or galactose. In some embodiment, the production medium further comprises or is further supplemented with zinc. In some embodiments, the production medium further comprises or is further supplemented with lysine and/or arginine. In some embodiments, the cell culture is cultivated for 5-20 days. In some embodiments, the cell culture is cultivated for 10-16 days. In some embodiments, the cell culture is cultivated for 13-15 days. In some embodiments, the cell culture is cultivated for 5, 6, 7, 8, 9, 10, 11, 12, 13, 14, 15, 16, 17, 18, 19 or 20 days. Also provided herein is an anti-α4β7 antibody that is obtained by, or obtainable by, the foregoing methods.

In one embodiment, provided herein is a composition comprising vedolizumab having 88% or more, 90% or more, 91% or more, 92% or more, 93% or more, 94% or more, or 95% or more total asialo-, agalacto, core fucosylated biantennary glycan (G0F), asialo-, monogalacto, core fucosylated biantennary glycan (G1F), and/or asialo-, digalacto, core fucosylated biantennary glycan (G2F) glycosylation variants. In one embodiment, provided herein is a composition comprising vedolizumab having 91-96%, 92-95%, 91- 92%, 91-92.5%, 91-93%, or 91-95% total asialo-, agalacto, core fucosylated biantennary glycan (G0F), asialo-, monogalacto, core fucosylated biantennary glycan (G1F), and/or asialo-, digalacto, core fucosylated biantennary glycan (G2F) glycosylation variants. In some embodiments, the foregoing compositions are obtainable by culturing a GS-CHO cell recombinantly expressing vedolizumab in a production medium supplemented with uridine, manganese, and galactose. In some embodiments, the foregoing compositions are obtainable by culturing a GS-CHO cell recombinantly expressing vedolizumab in a production medium supplemented with uridine, manganese, galactose and zinc. In some embodiments, the foregoing compositions are obtainable by culturing a GS-CHO cell recombinantly expressing vedolizumab in a production medium supplemented with uridine, manganese, galactose, zinc, arginine, and/or lysine.

In one embodiment, provided herein is a composition comprising vedolizumab having 85% or less, 80% or less, 75% or less, 70% or less, 69% or less, 68% or less, 67% or less, 66% or less, 65% or less, 64% or less, 63% or less, 62% or less, 61% or less, 60% or less, 59% or less, 58% or less, 57% or less, 56% or less, or 55% or less asialo-, agalacto, core fucosylated biantennary glycan (G0F) (as determined by HILIC). In one embodiment, provided herein is a composition comprising vedolizumab having 45-65%, or 50-60% asialo-, agalacto, core fucosylated biantennary glycan (G0F) (as determined by HILIC). In some embodiments, the foregoing compositions are obtainable by culturing a GS-CHO cell recombinantly expressing vedolizumab in a production medium supplemented with uridine, manganese, and galactose. In some embodiments, the foregoing compositions are obtainable by culturing a GS-CHO cell recombinantly expressing vedolizumab in a production medium supplemented with uridine, manganese, galactose and zinc. In some embodiments, the foregoing compositions are obtainable by culturing a GS-CHO cell recombinantly expressing vedolizumab in a production medium supplemented with uridine, manganese, galactose, zinc, arginine, and/or lysine.

In one embodiment, provided herein is a composition comprising vedolizumab having 10% or more, 15% or more, 20% or more, 21% or more, 22% or more, 23% or more, 24% or more, 25% or more, 26% or more, 27% or more, 28% or more, 29% or more, 30% or more, 31% or more, 32% or more, or 33% or more asialo-, monogalacto, core fucosylated biantennary glycan (G1F) (as determined by HILIC). In one embodiment, provided herein is a composition comprising vedolizumab having 25-45%, or 30-40% asialo-, monogalacto, core fucosylated biantennary glycan (G1F) (as determined by HILIC). In some embodiments, the foregoing compositions are obtainable by culturing a GS-CHO cell recombinantly expressing vedolizumab in a production medium supplemented with uridine, manganese, and galactose. In some embodiments, the foregoing compositions are obtainable by culturing a GS-CHO cell recombinantly expressing vedolizumab in a production medium supplemented with uridine, manganese, galactose and zinc. In some embodiments, the foregoing compositions are obtainable by culturing a GS-CHO cell recombinantly expressing vedolizumab in a production medium supplemented with uridine, manganese, galactose, zinc, arginine, and/or lysine.

In one embodiment, provided herein is a composition comprising vedolizumab having 0.5% or more, 1% or more, 1.5% or more, 2% or more, 2.5% or more, 3% or more, 3.5% or more, 4% or more, 4.5% or more, 5% or more, 5.5% or more, 6% or more, 6.5% or more, 7% or more, or 8% or more asialo-, digalacto, core fucosylated biantennary glycan (G2F) (as determined by HILIC). In one embodiment, provided herein is a composition comprising vedolizumab having 2-4%, 3-5%, or 2-7% asialo-, digalacto, core fucosylated biantennary glycan (G2F) (as determined by HILIC). In some embodiments, the foregoing compositions are obtainable by culturing a GS-CHO cell recombinantly expressing vedolizumab in a production medium supplemented with uridine, manganese, and galactose. In some embodiments, the foregoing compositions are obtainable by culturing a GS-CHO cell recombinantly expressing vedolizumab in a production medium supplemented with uridine, manganese, galactose and zinc. In some embodiments, the foregoing compositions are obtainable by culturing a GS-CHO cell recombinantly expressing vedolizumab in a production medium supplemented with uridine, manganese, galactose, zinc, arginine, and/or lysine.

In some embodiments, a method for producing an anti-α4β7 antibody in a CHO cell culture comprises providing to the culture a medium supplement comprising a metal ion and a metal cofactor and another medium supplement comprising a nucleoside and a sugar. In some embodiments, a method for producing an anti-α4β7 antibody in a CHO cell culture comprises providing to the culture a medium supplement comprising a metal ion and a medium supplement comprising a nucleoside, a sugar and a metal cofactor. In some embodiments, a method for producing an anti-α4β7 antibody in a CHO cell culture comprises providing to the culture a medium supplement comprising a metal ion, a nucleoside, a sugar and a metal cofactor. In some embodiments, a method for producing an anti-α4β7 antibody in a CHO cell culture comprises providing to the culture a medium supplement comprising a metal ion, a nucleoside and a metal cofactor. Exemplary medium supplements and their methods of use provided in the

Examples are considered embodiments of the invention.

C. Supplementation with Lysine and/or Arginine

In some embodiments of the foregoing aspects, the cell culture medium, e.g., production phase medium, can be further supplemented with lysine and/or arginine.

Accordingly, in some aspects, the methods and compositions provided herein can employ a cell culture medium, e.g., production phase medium, that is supplemented with, zinc, lysine, and/or arginine. In other aspects, the methods and compositions provided herein can employ a cell culture medium, e.g., production phase medium, that is supplemented with uridine, manganese, galactose, lysine, and/or arginine. In other aspects, the methods and compositions provided herein can employ a cell culture medium, e.g., production phase medium, that is supplemented with uridine, manganese, galactose, zinc, lysine, and/or arginine.

In one embodiment, the production medium comprises 5.0 to 8.8 g/L lysine and 3.0 to 12.0 g/L arginine. In one embodiment, the production medium comprises 4.5 to 5.5 g/L lysine. In one embodiment, the production medium comprises 5.5 to 8.8 g/L lysine. In one embodiment, the production medium comprises 5.4 to 7.4 g/L arginine. In one embodiment, the production medium comprises 7.4 to 12 g/L arginine.

Medium can be supplemented with uridine, manganese, galactose, and zinc as described above. For example, in some embodiments, the cell culture medium, e.g., production phase medium, is supplemented with 0.1-20 mM uridine, 0.1-100 μM manganese, 0.1-100 mM galactose, and 1-100 μM zinc, and is further supplemented with 5.0-8.8 g/L lysine and/or 3.0 to 12.0 g/L arginine.

III. Upstream Production Methods

The present invention concerns the large-scale recombinant production of an antibody, such as anti-α4β7 antibodies in mammalian host cells under conditions and/or with supplements identified herein that result in an anti-α4β7 antibody, such as vedolizumab, titer greater than 3 g/L. High levels of recombinant antibody expression in mammalian cell culture systems is a known challenge in the art.

The overall process includes inoculation of cell culture medium with mammalian cells genetically modified to express the anti-α4β7 antibody, a growth phase, a production phase, and finally a harvesting stage whereby the recombinant antibody is collected. In between the various stages may, in certain embodiments, be transition stages.

Thus, as a first step, the nucleic acid (e.g., cDNA) encoding the desired recombinant anti-α4β7 antibody may be inserted into a replicable vector for expression. Various vectors are publicly available and known to those in the art. The vector components generally include, but are not limited to, one or more of the following: a signal sequence, an origin of replication, one or more marker genes, an enhancer element, a promoter, and a transcription termination sequence, each of which is described below. Optional signal sequences, origins of replication, marker genes, enhancer elements and transcription terminator sequences that may be employed are known in the art and described in further detail in PCT Publication WO 97/25428 or U.S. Pat. No. 7,053,202.

Expression vectors usually contain a promoter that is recognized by the host organism and is operably linked to the protein-encoding nucleic acid sequence. Promoters are untranslated sequences located upstream (5′) to the start codon of a structural gene (generally within about 100 to 1000 bp) that control the transcription and translation of a particular nucleic acid sequence to which they are operably linked. Such promoters typically fall into two classes, inducible and constitutive. Inducible promoters are promoters that initiate increased levels of transcription from DNA under their control in response to some change in culture conditions, e.g., the presence or absence of a nutrient or a change in temperature. At this time a large number of promoters recognized by a variety of potential host cells are well known. These promoters are operably linked to DNA encoding the desired protein by removing the promoter from the source DNA by restriction enzyme digestion and inserting the isolated promoter sequence into the vector.

Expression vectors that provide for the transient expression in mammalian cells may be employed. In general, transient expression involves the use of an expression vector that is able to replicate efficiently in a host cell, such that the host cell accumulates many copies of the expression vector and, in turn, synthesizes high levels of a desired polypeptide encoded by the expression vector (Sambrook et al., supra). Transient expression systems, comprising a suitable expression vector and a host cell, allow for the convenient positive identification of polypeptides encoded by cloned DNAs, as well as for the rapid screening of such polypeptides for desired biological or physiological properties. Mammalian host cells are transfected and preferably transformed with expression vectors and cultured in nutrient media modified as appropriate for inducing promoters, selecting transformants, or amplifying the genes encoding the desired sequences. Such cells are then allowed to grow and eventually, having undergone several rounds of replication, are transferred to a larger container for subsequent growth and eventual production of the polypeptide of interest.

Mammalian cells, such as CHO cells, may be cultured in small scale cultures, e.g., up to 5 L, such as for example, in 5 ml, 25 ml, 50 ml, 100 ml, 250 ml, 1 L, 3 L or 5 L containers. Alternatively, the cultures can be mid-size scale containers, such as, for example 10 L, 20 L, 100 L or 200 L containers. Alternatively, cultures may be large scale cultures in vessels greater than 200 L, such as 500 L, 1000 L, 2000 L 3000 L, 5000 L 10,000 L, and 15,000 L vessels. Large scale cell cultures, such as for manufacturing of a therapeutic antibody, are typically maintained for days, or even weeks, while the cells produce the desired protein(s).

For the purposes of this invention, cell culture medium is a medium suitable for growth of animal cells, such as mammalian cells, in in vitro cell culture. Examples of types of cell culture media include expansion cell culture media and production cell culture media.

Cell culture media formulations are well known in the art. Typically, cell culture media are comprised of buffers, salts, carbohydrates, amino acids, vitamins and trace essential elements. The cell culture medium may or may not contain serum, peptone, protein hydrolysates and/or proteins. Various tissue culture media, including serum-free and defined culture media, are commercially available, for example, any one or a combination of the following cell culture media can be used: RPMI-1640 Medium, RPMI-1641 Medium, Dulbecco's Modified Eagle's Medium (DMEM), Minimum Essential Medium Eagle, F-12K Medium, Ham's F12 Medium, Iscove's Modified Dulbecco's Medium, McCoy's 5A Medium, Leibovitz's L-15 Medium, and serum-free media such as EX-CELL.TM. 300 Series (JRH Biosciences, Lenexa, Kans.), among others. Cell culture media may be supplemented with additional or increased concentrations of components such as amino acids, salts, sugars, vitamins, hormones, growth factors, buffers, antibiotics, lipids, trace elements and the like, depending on the requirements of the cells to be cultured and/or the desired cell culture parameters. CHO cell media are known in the art, e.g., CD-CHO (Invitrogen), CD-CHO-AGTTM medium (ThermoFisher Scientific),

HYCELLTM CHO medium (GE Healthcare Life Sciences) or CHOMACS CD medium (Militenyi Biotech). In some embodiments, a commercially available medium as described above, may be used as a starting medium for a production phase culture for producing an anti-α4β7 antibody, such as vedolizumab, e.g., in GS-CHO cells. In one preferred embodiment, the antibody is produced in GS-CHO cells grown in CD-CHO medium, wherein the CD-CHO medium is supplemented as described herein.

Prior to the production phase, mammalian cells are cultured first in a growth phase under environmental conditions that maximize cell proliferation and viability. Following the growth phase, the production phase is initiated, whereby cell culture conditions that maximize polypeptide production are used. The growth and production phases may be preceded by, or separated by, one or more transition phases. For example, in one embodiment, the production phase of the cell culture process is preceded by a transition phase of the cell culture in which parameters for the production phase of the cell culture are engaged. In the growth phase, mammalian cells are grown under conditions and for a period of time that is maximized for growth. Culture conditions, such as temperature, pH, dissolved oxygen (DO₂), and the like, are those used with the particular host and will be apparent to the ordinarily-skilled artisan. Generally, the pH is adjusted to a level between about 6.5 and 7.5 using either an acid (e.g., CO₂) or a base (e.g., Na₂CO₃ or NaOH). A suitable temperature range for culturing mammalian cells such as CHO cells is between about 30 to 40 degrees Celsius and preferably ranging from 36 to 38 degrees Celsius.

In a commercial process for production of a protein by mammalian cells, there are commonly multiple, for example, at least about 2, 3, 4, 5, 6, 7, 8, 9, or 10 growth phases that occur in different, e.g., successively larger, culture vessels preceding a final production phase.

When the cells grow to sufficient numbers, they are transferred to large-scale production containers, e.g., bioreactors, to begin the production phase whereby the mammalian host cells are cultured under conditions that promote the production of the polypeptide of interest, i.e., an antibody. The skilled artisan may choose to use one or more of cell culture media described herein that have been developed to recombinant polypeptide production in a particular cultured host cell. Alternatively, the methods and compositions according to the current invention may be used in combination with commercially available cell culture media. Typically the growth phase occurs at a higher temperature than a production phase.

For example, a growth phase may occur at a first temperature from about 35 degrees Celsius to about 38 degrees Celsius, and a production phase may occur at a second temperature from about 30 degrees Celsius to about 34 degrees Celsius. As described in the examples, however, one of the improvements identified herein is that maintaining a substantially similar temperature between the growth phase and the production phase of mammalian cells in a cell culture for the production of an anti-α4β7 antibody, e.g., vedolizumab, provides for increased antibody titer from the cell culture. Indeed, by maintaining a similar temperature between the two phases, antibody titer of vedolizumab was greater than 1 g/L, e.g., about 5 to 7 g/L.

Thus, in one embodiment, the invention features a method of producing a humanized anti-α4β7 antibody in mammalian host cells, where the mammalian host cells are cultured in a cell culture medium in an expansion phase, and subsequently cultured in a cell culture medium in a production phase where both the expansion and the production phases are performed at about the same average temperature, e.g., an average temperature of both phases from 36 to 38 degrees Celsius. In one embodiment, the average temperature of both the expansion and the production phase is from 36.5 to 37.5 degrees Celsius, e.g., about 37 degrees Celsius.

Alternatively, the invention features a method of producing a humanized anti-α4β7 antibody in mammalian host cells, where the mammalian host cells are cultured in a cell culture medium in an expansion phase, and subsequently cultured in a cell culture medium in a production phase, where both the expansion and the production phases are performed at about the same temperature range, e.g., from 36 to 38 degrees Celsius, e.g., any temperature ranging from 36.5 to 37.5 degrees Celsius.

The length of the production phase may vary depending on the cells and the antibody being expressed. In certain embodiments, the production phase is about 14 days or less. In certain embodiments, the production phase is about 15 days or less. In certain embodiments, the production phase is about 16 days or less. Alternatively, the production phase is 8 days, 9 days, 10 days, 11 days, 12 days, 13 days, 14 days, 10 to 16 days, 11 to 15 days, 13 to 17 days, or 12 to 14 days. Included within these numbers are partial days, e.g.,13.5 days.

In one embodiment, the pH of the cell culture medium ranges from 6.0 to 8.0; 6.5 to 7.5; 6.7 to 7.0, 6.7 to 6.9, 6.95-7.05, or 7.1 to 7.2. Numbers intermediate to these pH values, e.g., 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, and 8.0, as well as all other numbers recited herein, are also intended to be part of this invention. Ranges of values using a combination of any of the above recited values as upper and/or lower limits are intended to be included in the scope of the invention. In some embodiments, the pH of a culture may shift from one pH to another, such as to a lower pH than at inoculation. For example, the pH may shift from a pH range of 6.9 to 7.1, 6.95 to 7.05 or pH 7.00 ±0.1, ±0.05 or ±0.02 to a pH range of 6.7 to 7.0, 6.75 to 6.85 or pH 6.8 ±0.1 or ±0.02. The timing of the shift may be after 2, 3, 4 or 5 days in culture. In some embodiments, the pH shift is at day four or day five of the production phase culture.

Accordingly, in one embodiment, provided herein is a method of producing a humanized anti-α4β7 antibody in mammalian host cells genetically engineered to express the antibody, where the mammalian host cells are cultured in a production medium at a first pH, and are subsequently shifted to a second pH, wherein the second pH is lower than the first pH. For example, in some embodiments, the second pH may be shifted 0.1 to 0.5 pH units lower than the first pH during the production phase of the host cell culture. In one embodiment, the starting pH can be in the range of pH 6.8 to pH 7.2. After the pH shift occurs, the adjusted pH can be reduced by 0.1 to 0.5 pH units, e.g., 0.1, 0.2, 0.3, 0.4, or 0.5 pH units. Accordingly, in some embodiments, the second pH can be in the range of about 6.7-6.95.

As described in the examples below, a pH shift during the production phase can, for example, reduce the amount of basic isoform of the antibody, reduce the amount of acidic isoform of the antibody, and/or increase the amount of major isoform of the antibody.

In certain embodiments, the pH of the cell culture medium during the production phase is maintained within a pH range of 6.5 to 7.0.

In certain embodiments, the pH of the cell culture medium during the production phase is maintained within a pH range of 6.7 to 7.0.

In certain embodiments, the pH of the cell culture medium during the production phase is about 6.85.

During the production phase time the culture can be supplemented with a concentrated feed medium containing components, such as nutrients and amino acids, which are consumed during the course of the production phase of the cell culture.

Concentrated feed medium may be based on just about any cell culture media formulation. Such a concentrated feed medium can contain most, or a subset, of the components of the cell culture medium at, for example, about 5×, 6×, 7×, 8×, 9×, 10×, 12×, 14×, 16×, 20×, 25 to 40×, 30×, 50×, 100×, 40 to 120×, 200×, 400×, 600×, 800×, or even about 1000×, of their normal amount. Concentrated feed media are often used in fed batch culture processes.

In one embodiment, the production phase is a fed batch culture. Fed batch culture is a widely-practiced culture method for large scale production of proteins from mammalian cells. See e.g. Chu and Robinson (2001), Current Opin. Biotechnol. 12: 180-87. Antibody production can be demanding for cells and the base or starting medium cannot sustain high density of cells and high levels of antibody production. Without fresh nutrients, such as amino acids or energy sources, the yield may suffer or the cells may die. For example, a culture that consumes its supply of amino acids, such as tyrosine, will stop producing antibodies. A fed batch culture of mammalian cells is one in which the culture is fed, either continuously or periodically, with a concentrated feed medium containing nutrients. Feeding can occur on a predetermined schedule of, for example, every day, once every two days, once every three days, etc. In one embodiment, one or more additional nutrients, e.g., selected from the group consisting of glucose, zinc, manganese, uridine, and galactose, is added to a cell culture medium, e.g., in a medium supplement, beginning on or about Day 4 of the production phase. The feed solution is added on a schedule that is daily, every other day, every two days, and combinations thereof. In some embodiments, tyrosine is added in a bolus twice during the production phase, such as on day 4 and day 11. In other embodiments, tyrosine is added daily, e.g., in a feed supplement, to a production phase culture. In some embodiments, glucose is added to a production phase culture. In some embodiments, glucose consumption is monitored, e.g., by measuring glucose or its metabolites, such as lactic acid. In some embodiments, a feed supplement comprising glucose is added so glucose levels are controlled at a level of 1 to 10 g/L, 2 to 7 g/L, 2.5 to 6 g/L or about 7 g/L.

In certain embodiments, a fed batch method is used in the expansion phase of the mammalian cell culture process to supplement the growing cells.

In a particular embodiment the cell culture of the present invention is performed in a large scale bioreactor and a fed-batch culture procedure is employed. In one embodiment of a fed-batch culture, the mammalian host cells and culture medium are supplied to a culturing vessel initially and additional culture nutrients are fed, continuously or in discrete increments, to the culture during culturing, with or without periodic cell and/or product harvest before termination of culture. The fed-batch culture can include, for example, a semi-continuous fed-batch culture, wherein periodically whole culture (including cells and medium) is removed and replaced by fresh medium fed-batch culture is distinguished from simple-batch culture in which all components for cell culturing (including the cells and all culture nutrients) are supplied to the culturing vessel at the start of the culturing process.

The methods described herein can be used to achieve a cell culture having a titer of the humanized anti-α4β7 antibody of greater than 1 g/L. In one embodiment, the methods described herein are used to achieve a titer of humanized anti-α4β7 antibody of about 2 to about 6 g/L, about 3 to about 5 g/L, about 5 to about 9 g/L or about 4.5 to about 7 g/L.

The methods disclosed herein can be used to achieve antibody compositions having certain glycosylation patterns. In one embodiment, the methods described herein provide a population of humanized anti-α4β7 antibodies where the population has 88% or more, 90% or more, or 91% or more, total asialo-, agalacto, core fucosylated biantennary glycan (G0F), asialo-, monogalacto, core fucosylated biantennary glycan (G1F), and/or asialo-, digalacto, core fucosylated biantennary glycan (G2F) glycosylation variants.

The methods disclosed herein can also be used to achieve an antibody composition having a certain amount of major isoform of the antibody. In one embodiment, the methods disclosed herein provide a composition (e.g., a clarified harvest comprising vedolizumab) having a major antibody isoform amount greater than or equal to 61% as determined by Cation Exchange Chromatography (CEX). In another embodiment, the methods disclosed herein provide a composition (e.g., a clarified harvest comprising vedolizumab) having a major antibody isoform amount greater than or equal to 62% as determined by CEX. In one embodiment, the methods disclosed herein provide a composition (e.g., a clarified harvest comprising vedolizumab) having a major antibody isoform amount greater than or equal to 63% as determined by CEX. In one embodiment, the methods disclosed herein provide a composition (e.g., a clarified harvest comprising vedolizumab) having a major antibody isoform amount greater than or equal to 64% as determined by CEX. In one embodiment, the methods disclosed herein provide a composition (e.g., a clarified harvest comprising vedolizumab) having a major antibody isoform amount greater than or equal to 65% as determined by CEX.

### IV. Downstream Production Methods

Compositions comprising an anti-α4β7 antibody or antigen binding portion thereof, e.g., vedolizumab, of the invention can be produced by the upstream cell culture methods and compositions provided herein. These upstream process technologies can optionally be coupled with downstream production methods for isolating, purifying, and/or formulating the antibody, or antigen binding portion thereof. Following the production phase, the recombinant antibody can be harvested. Typically, the mammalian cells are engineered to secrete the protein of interest into the cell culture media, so the first step in the purification process is to separate the cells from the media. The harvested media can be further clarified, e.g., by filtration. The media, e.g., clarified harvest can then be subjected to several additional purification steps that remove any cellular debris, unwanted proteins, salts, minerals or other undesirable elements. The recombinant antibody can be purified from contaminant soluble proteins and polypeptides, with the following procedures being exemplary of suitable purification procedures, that can include one or more of the following: affinity chromatography, e.g. using a resin that binds an Fc region of an antibody, such as Protein A; fractionation on an ion-exchange column or resin such as cation exchange chromatography (CEX), e.g., SP-Sepharose™ or CM-Sepharose™ hydroxyapatite; anion exchange chromatography (AEX); hydrophobic interaction chromatography (HIC); mixed mode chromatography; ethanol precipitation; chromatofocusing; ammonium sulfate precipitation; gel filtration using, for example, Sephadex G-75™; ultrafiltration and/or diafiltration, or combinations of the foregoing. Examples of purification methods are described in Liu et al., mAbs, 2:480-499 (2010). At the end of the purification process, the recombinant protein is highly pure and is suitable for human therapeutic use, e.g., in pharmaceutical antibody formulations described below. Following purification, the highly pure recombinant protein may be ultrafilered/diafiltered (UF/DF) into a pharmaceutical formulation suitable for human administration.

Following diafiltration and ultrafiltration, the antibody formulation may remain as a liquid or be lyophilized into a dry antibody formulation. In one aspect, the dry, lyophilized antibody formulation is provided in a single dose vial comprising 150 mg, 180 mg, 240 mg, 300 mg, 360 mg, 450 mg or 600 mg of anti-α4β7 antibody and can be reconstituted with a liquid, such as sterile water, for administration. In another aspect, the anti-α4β7 antibody, e.g., vedolizumab, is in a stable liquid pharmaceutical composition stored in a container, e.g., a vial, a syringe or cartridge, at about 2-8° C. until it is administered to a subject in need thereof. In some embodiments, the reconstituted lyophilized formulation or the stable liquid pharmaceutical composition of anti-α4β7 antibody comprises about 0% to 5.0%, 0% to 2%, ≤2%, ≤1%, ≤0.6% or ≤0.5% aggregates.

Accordingly, in some embodiments, provided herein is a reconstituted lyophilized antibody formulation or a stable liquid pharmaceutical composition comprising a humanized anti-α4β7 antibody, or antigen binding portion thereof. In some embodiments, the reconstituted lyophilized formulation or the stable liquid pharmaceutical composition of anti-α4β7 antibody comprises about 11% to 16%, 12% to 15%, ≤14%, ≤13%, ≤12%, or ≤11% basic isoform species. In some embodiments, the reconstituted lyophilized formulation or the stable liquid pharmaceutical composition of anti-α4β7 antibody comprises 65% to 75%, 66% to 74%, 67% to 73%, at least 65%, at least 66%, at least 67%, at least 68%, at least 69%, or at least 70% major isoform. In some embodiments, the reconstituted lyophilized formulation or the stable liquid pharmaceutical composition of anti-α4β7 antibody comprises a total asialo-, agalacto, core fucosylated biantennary glycan (G0F), asialo-, monogalacto, core fucosylated biantennary glycan (G1F), and/or asialo-, digalacto, core fucosylated biantennary glycan (G2F) glycosylation variant (G0F+G1F+G2F) content of 92% to 98%, 92% to 97%, 92% to 96%, 92% to 95%, at least 92%, at least 93%, at least 94%, or at least 95%. In some embodiments, the reconstituted lyophilized formulation or the stable liquid pharmaceutical composition of anti-α4β7 antibody comprises a G0F content of 45% to 65%, 50% to 65%, 55% to 65%, 45% to 60%, 50% to 60%, 55% to 60%, 45% to 55%, 47% to 61%, 47% to 63%, 65% or less, 64% or less, 63% or less, 62% or less, 61% or less, 60% or less, 57% or less, 55% or less, 53% or less, 52% or less or 50% or less. In some embodiments, the reconstituted lyophilized formulation or the stable liquid pharmaceutical composition of anti-α4β7 antibody comprises a G1F content of 25% to 45%, 26% to 42%, 27% to 40%, 30% to 40%, 30% to 45%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, or at least 43%. In some embodiments, the reconstituted lyophilized formulation or the stable liquid pharmaceutical composition of anti-α4β7 antibody comprises a G2F content of 2% to 8%, 2.5% to 7.5%, 3% to 7%, 3.5% to 6.5%, at least 2%, at least 2.5%, at least 3%, at least 3.5%, at least 4%, at least 4.5%, at least 5%, or at least 5.5%, at least 6%, at least 6.5%, or at least 7%.

In some embodiments, the reconstituted lyophilized formulation or the stable liquid pharmaceutical composition of anti-α4β7 antibody can comprise one or more excipients, including but not limited to an amino acid (e.g., arginine, histidine, and/or histidine monohydrochloride), a sugar (e.g., sucrose), a surfactant (e.g., polysorbate 80), and/or a buffer (e.g., citrate, phosphate, etc.). In one embodiment, the reconstituted lyophilized formulation or the stable liquid pharmaceutical composition of anti-α4β7 antibody comprises L-arginine, L-histidine, L-histidine monohydrochloride, sucrose, and/or polysorbate 80. In another embodiment, the reconstituted lyophilized formulation or the stable liquid pharmaceutical composition of anti-α4β7 antibody comprises citrate, arginine, histidine, and/or polysorbate 80.

The syringe or cartridge may be a 1 mL or 2 mL container (for example for a 160 mg/mL dose) or more than 2 ml, e.g., for a higher dose (at least 320 mg or 400 mg or higher). The syringe or cartridge may contain at least about 20 mg, at least about 50 mg, at least about 70 mg, at least about 80 mg, at least about 100 mg, at least about 108 mg, at least about 120 mg, at least about 155 mg, at least about 180 mg, at least about 200 mg, at least about 240 mg, at least about 300 mg, at least about 360 mg, at least about 400 mg, or at least about 500 mg of anti-α4β7 antibody. In some embodiments, the container, e.g., syringe or cartridge may be manufactured to deliver about 20 to 120 mg, about 40 mg to 70 mg, about 45 to 65 mg, about 50 to 57 mg or about 54 mg of anti-α4β7 antibody, e.g., vedolizumab. In other embodiments, the syringe or cartridge may be manufactured to deliver about 90 to 120 mg, about 95 to 115 mg, about 100 to 112 mg or about 108 mg of anti-α4β7 antibody, e.g., vedolizumab. In other embodiments, the syringe or cartridge may be manufactured to deliver about 140 to 250 mg, about 150 to 200 mg, about 160 to 170 mg, about 160 to 250 mg, about 175 mg to 210 mg, about 220 to 260 mg, or about 160 mg, about 165 mg, about 180 mg or about 200 mg of anti-α4β7 antibody, e.g., vedolizumab.

Administration of a formulation can be by parenteral injection such as intravenous, subcutaneous or intramuscular. An intravenous injection can be by infusion, such as by further dilution with sterile isotonic saline, buffer, e.g., phosphate-buffered saline or

Ringer's (lactated or dextrose) solution. In some embodiments, the anti-α4β7 antibody is administered by subcutaneous injection, e.g., a dose of about 54 mg, 108 mg or about 165 mg or about 216 mg, at about every two, three or four weeks after the start of therapy or after the third subsequent dose.

### V. Analytical Methods

Various parameters of an antibody, or antigen binding portion thereof, reported herein can be measured using standard analytical methods and techniques, such as those described below.

In various embodiments set forth herein, cation exchange chromatography (CEX) can be used to determine the relative amounts of the major isoform, basic isoform(s), and acidic isoform(s) present in a population of an antibody or antigen binding portion thereof, e.g., vedolizumab. The CEX method fractionates antibody species according to overall surface charge. After dilution to low ionic strength using mobile phase, the test sample can be injected onto a CEX column, such as for example a Dionex Pro-Pac™ WCX-10 column (Thermo Fisher Scientific, Waltham, Mass. (USA)), equilibrated in a suitable buffer, e.g., 10 mM sodium phosphate, pH 6.6. The antibody can be eluted using a sodium chloride gradient in the same buffer. Protein elution can be monitored at 280 nm, and peaks are assigned to acidic, basic, or major isoforms categories. Acidic peaks elute from the column with a shorter retention time than the major isoform peak, and basic peaks elute from the column with a longer retention time than the major isoform peak. The percent major isoform, the sum of percent acidic species, and the sum of percent basic species are reported. The major isoform retention time of the sample is compared with that of a reference standard to determine the conformance.

In one embodiment, a CEX assay method comprises diluting a test sample to low ionic strength, injecting onto a CEX column which is equilibrated in 10 mM sodium phosphate, pH 6.6, eluting the column with a NaCl gradient in this buffer, monitoring the peaks at 280 nm and assigning peaks as acidic, main or basic, wherein the acidic peaks elute first with the shortest retention times, the main peak elutes second and the basic peaks elute with the longest retention times, and the peak areas are quantified and their amounts are calculated as the percent of all the peak area.

In various embodiments set forth herein, hydrophilic interaction phase separation (HILIC) can be used to determine the glycoform profile of an antibody or antigen binding portion thereof, e.g., vedolizumab. The HILIC method fractionates free fluorescently- labeled carbohydrates. Intact glycans can be released from a sample of the antibody or antigen binding portion thereof by digestion with N-glycosidase F. Released glycans can then be immediately labeled with a fluorescent tag, such as InstantAB fluorescent tag, using standard techniques, such as those used in the GlykoPrep Rapid Glycoprotein Sample Preparation System from Prozyme (Hayward, Calif, (USA)). The labeled glycans can be fractionated using ultra performance liquid chromatography. In some embodiments, labeled glycans are fractionated using an ACQUITY UPLC BEH Amide Column (Waters Corporation, Milford, Mass. (USA)) and an acetonitrile/ammonium formate gradient system. Labeled glycans can be detected by fluorescence emission at 344 nm using an excitation wavelength of 278 nm. Thus, HILIC as used in connection with the invention is a HILIC method which fractionates free fluorescently-labeled glycoforms, wherein preferably, intact glycoforms are released from a sample of the antibody or antigen binding portion thereof by digestion with N-glycosidase F, the released glycoforms are then immediately labeled with a fluorescent tag, preferably InstantAB fluorescent tag, using standard labeling techniques, preferably those used in the GlykoPrep Rapid Glycoprotein Sample Preparation System from Prozyme (Hayward, Calif. (USA)), wherein the labeled glycoforms are fractionated using ultra performance liquid chromatography, preferably using an ACQUITY UPLC BEH Amide Column (Waters Corporation, Milford, Mass. (USA)) and an acetonitrile/ammonium formate gradient system, and wherein the labeled glycoforms are detected by fluorescence emission at 344 nm using an excitation wavelength of 278 nm. An assay control can be performed by confirming appropriate resolution of commercially available standards, e.g., InstantAB-labeled glucose homopolymer ladder (Agilent Technologies, Inc., Santa Clara, Calif. (USA)). The quantitation is based on the relative area percent of detected sugars. The percent peak area of the G0F (asialo-, agalactosylated biantennary glycan, core fucosylated); G1F (asialo-, monogalactosylated biantennary glycan, core fucosylated); and G2F (asialo-, digalactosylated biantennary glycan, core fucosylated) species are reported.

In various embodiments set forth herein, size exclusion chromatography (SEC) can be used to determine the relative level of monomers, high molecular weight (HMW) aggregates, and low molecular weight (LMW) degradation products present in a population of an antibody or antigen binding portion thereof, e.g., vedolizumab. The SEC method provides size-based separation of antibody monomer from HMW species and LMW degradation products. Test samples and reference standards can be analyzed using commercially available SEC columns, using an appropriate buffer. For example, in some preferred embodiments, SEC analysis can be performed using a G3000 SWx1 column (Tosoh Bioscience, King of Prussia, Pa. (USA)), or preferably two G3000 SWx1 columns connected in tandem, and an isocratic phosphate-sodium chloride buffer system, pH 6.8. Elution of protein species is monitored at 280 nm. The main peak (monomer) and the total peak area are assessed to determine purity. In one embodiment, the SEC analysis comprises injecting a sample onto two G3000 SWxl columns connected in tandem, and run in an isocratic phosphate-sodium chloride buffer system, pH 6.8, wherein the elution of protein species is monitored at 280 nm and the main peak (monomer) and the total peak area are measured. The purity (%) of the sample (calculated as % monomer), the % HMW aggregate, and/or the % LMW degradation product are reported.

Residual CHO host cell protein (HCP) impurities present in an antibody preparation can be measured if desired by enzyme-linked immunosorbent assay (ELISA), using standard techniques. Many ELISA kits designed for this purpose are commercially available, such as the CHO HCP ELISA Kit 3G from Cygnus Technologies (Southport, NC (USA)). Host cell proteins in a test sample can be captured using an immobilized polyclonal anti-CHO HCP antibody. Captured proteins can then be detected using a suitable detection agent, for example, a horseradish peroxidase-labeled version of the same antibody. In this exemplary embodiment, the amount of captured peroxidase, which is directly proportional to the concentration of CHO HCP, can be measured colorimetrically at 450 nm using the peroxidase substrate 3,3′,5,5′-tetramethylbenzidine (TMB). Accordingly, the CHO HCP assay comprises using a polyclonal anti-CHO HCP antibody to capture HCP, which is detected after binding a horseradish peroxidase-labeled version of the polyclonal anti-CHO HCP antibody which converts the peroxidase substrate 3,3′,5,5′-tetramethylbenzidine (TMB) to a substance that is quantified colorimetrically at 450 nm. The HCP concentration can determined by comparison to a CHO HCP standard curve, such as that included in the test kit, and is reported as a percentage of the total level of protein in the antibody preparation.

The following example exemplifies improved methods and compositions for producing antibodies in mammalian cell culture. The following example is offered for illustrative purposes only, and is not intended to limit the scope of the present invention in any way. Commercially available reagents referred to in the example were used according to manufacturer's instructions unless otherwise indicated.

### EXAMPLES

Vedolizumab previously was produced in a dihydrofolate reductase deficient (DHFR) Chinese hamster ovary (CHO) cell line (Urlaub and Chasin (1980) Proc. Natl. Acad. Sci. USA, 77:4216-4220, US Patent Application Publication No. 20070122404). While the selected clone was stable in its expression of vedolizumab, the production levels were less than 2 g/L. Given high demand for material, investigators sought to develop a higher producing cell line.

After testing various selection systems on thousands of clones, and evaluating some clones in bioreactors, a glutamine synthase deficient (GS-) Chinese Hamster Ovary (GS-CHO) cell line was chosen. In one example, the GS CHO system yielded 6.7 g/L antibody. Additional studies indicated that culture conditions and media supplements could influence certain quality attributes. The below examples describe experiments for improving the quality of vedolizumab produced in GS-CHO cells.

### Example 1

### Cell Culture Production Impact on Product Quality Attributes

To improve product quality attributes, a Plackett-Burman method was used to create a screening design to assess the impact of five process parameter modification factors in eight bioreactors runs. The cells were thawed and a standard scale-up strategy was used with 3-day passaging to move from shake flasks into a 3L production bioreactor with 1.75L working volume. A bolus feeding strategy with two feeds (unless otherwise specified) was used for a 15-day bioreactor production.
Design: The following five different factors were chosen for this screening study: 1) temperature shift (to 33° C.); 2) change in feeding strategy (2 g/L vs 6 g/L glucose); 3) pH change (6.85 vs 7.05); 4) Uridine, Manganese Chloride and Galactose (UMG) addition to feed solution; and 5) Sigma Gal+/ExCell® Glycosylation Adjust addition. These five factors were tested on eight different bioreactor runs based on a Plackett-Burman screening design, described in Table 1.

TABLE 1
Plackett-Burman design for improving
galactosylation and acidic variants

| | Factors | | | | |
|---|---|---|---|---|---|
| | | | | UMG | |
| | Temperature | Feeding | | addition | Sigma |
| Vessel No. | shift | strategy | Low pH | to feed | Gal+ |
| A1 V09 | Yes | Yes | Yes | No | Yes |
| B1 V10 | No | No | Yes | No | No |
| C1 V11 | No | Yes | No | Yes | No |
| D1 V12 | No | No | Yes | Yes | Yes |
| E1 V13 | Yes | No | No | No | No |
| F1 V14 | Yes | Yes | Yes | Yes | No |
| G1 V15 | Yes | No | No | Yes | Yes |
| H1 V16 | No | Yes | No | No | Yes |

Plackett-Burman screening design was made through JMP software. This screening design aided in identifying factors that contributed to achieving product quality targets. Each factor consisted of two levels, as explained in Table 2 below.

TABLE 2
Investigative factors

| Factors | Levels | Corresponding condition |
|---|---|---|
| Temperature shift | Yes | On Day 7 to 33° C. |
| | No | 37° C. (No shift) |
| Feeding strategy | Yes | 2 g/L + projected consumption for glucose |
| | No | 6 g/L + projected consumption for glucose |
| pH | Yes | 6.85 ± 0.15 |
| | No | 7.05 ± 0.15 |
| UMG | Yes | 100X* |
| | No | Not added |
| Sigma Gal+ | Yes | 0.3% v/v |
| | No | Not added |

*100X UMG~100 mM Uridine, 0.2 mM Manganese Chloride and 500 mM Galactose in feed solution added to production medium
GS-CHO cell line was used for this experiment.

Feeding: A production bioreactor culture was inoculated with 3×10⁵ viable cells/mL, and during day 4, a feed medium was added to the cultures based on the cells' growth rate and glucose consumption rate per study design. Dosing amount of feed was set to cap at 7 g/L of glucose concentration. A shift in temperature was initiated on day 7 from 37 degrees Celsius to 33 degrees Celsius or 35 degrees Celsius according to the study design. All production bioreactor cultures were harvested either on day 18 or when target cells viability of less than or equal to 50%, whichever came first.

Product Quality: The results of the conditions tested in Table 1 were analyzed in

JMP software in order to explore conditions (using a prediction profiler) that would improve product quality attributes.

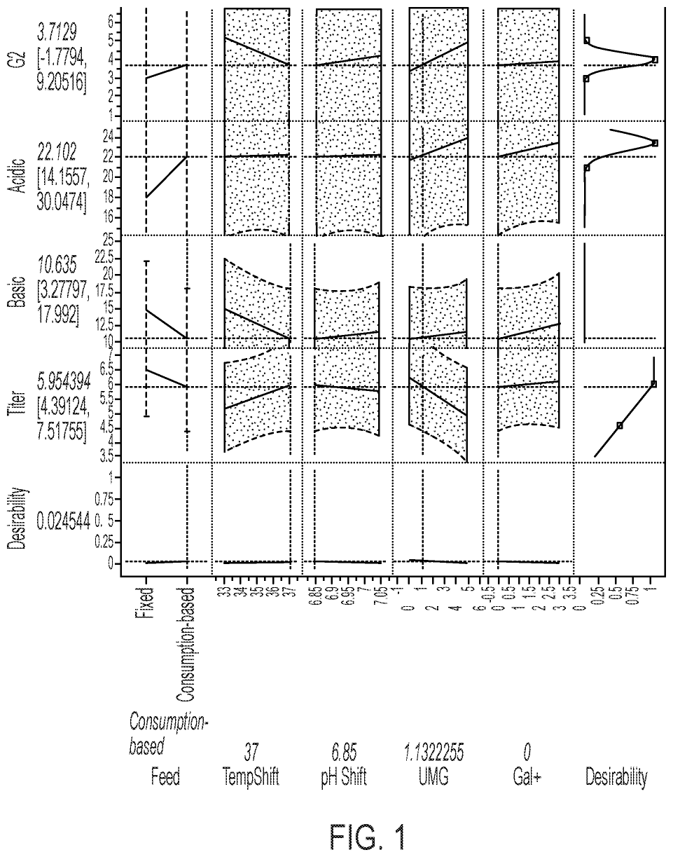

The prediction profile results are described in FIG. 1. Generally, conditions that achieve an increase in antibody titer, a decrease in basic and acidic species of vedolizumab, and an increase in G2F isoforms of vedolizumab are generally desirable.

As shown in FIG. 1, the model predicted that operating at Feed delivery based on glucose consumption rate, 37 degrees Celsius, shifting to pH 6.85, and UMG addition to the feed solution are optimal. In contrast, the results in FIG. 1 suggest that Gal+addition was not necessary as it had little impact on G2 isoforms, acidic or basic species of vedolizumab, or antibody titer. Further, the temperature shift to 33 degrees Celsius from 37 degrees Celsius had a negative impact on titer, suggesting maintaining cell production at 37 degrees was advantageous, whereas a pH of less than 7.05 (e.g., 6.85 to less than 7) improved titer while maintaining lower levels of G2 isoform.

The prediction profiler further showed advantages in using a UMG combination for achieving the carbohydrate target, as well as higher titer levels and lower levels of acidic species of the antibody. A glucose consumption-based feeding strategy and lower pH (6.85) as compared to pH 7 showed benefit in achieving lower basic species proportion.

### Example 2

### Effect of UMG Supplementation and pH on Product Quality Attributes

The objective of this experiment was to test the effect of pH and UMG levels in the feed solution on product quality attributes. This experiment is a follow-up from Example 1. Design: GS-CHO cells were used for this experiment. The experiment was designed to accommodate a full factorial of pH, surveyed at 6.85 or 7.05, and UMG as a feed supplement (during production) surveyed at 33×, 50× and 66× concentrations. An additional condition with a pH shift on day 4 was added (V10). On Day 1 of the experiment, the titrant pump of V01 grossly overpumped titrant due to a loose pH probe connection, and the reactor had to be taken down. Since V10 had similar media and cells, the run template of V10 was quickly replaced to reflect that of V01. No temperature-shift was employed because no benefit was predicted, as described in Example 1.

The cells were fed using the consumption-based feeding method, where the current growth rate and consumption rate is extrapolated to predict forward the glucose requirement.

The experiment assessed the effects of UMG supplementation in the feed medium (surveyed at 33× (33 mM uridine, 0.066 mM manganese, and 165 mM galactose), 50× (50 mM uridine, 0.1 mM manganese, and 250 mM galactose) and 66× (66 mM uridine, 0.132 mM manganese, and 330 mM galactose) concentrations in the feed medium) and pH (surveyed at 7.05 and 6.85) on product quality attributes, including the amount of antibody titer, acidic species, basic species, major species, G0F species, G1F species, G2F species, and glycan summation. Results were compared to those cultures grown in CD-CHO production media without UMG supplementation. The experimental design is described in Table 3.

TABLE 3
Experimental design

| Vessel | pH | Other | UMG addition | Issues |
|---|---|---|---|---|
| V01 | 6.85 | N/A | 15 mL 50X UMG | Eliminated due to |
| | | | | overpumping of |
| | | | | titrant |
| V02 | 6.85 | N/A | 15 mL 33X UMG | |
| V03 | 6.85 | N/A | 15 mL 66X UMG | |
| V04 | 6.65 | N/A | 15 mL 50X UMG | |
| V05 | 7.05 | N/A | 15 mL 50X UMG | |
| V06 | 7.05 | N/A | 15 mL 33X UMG | |
| V09 | 7.05 | N/A | 15 mL 66X UMG | |
| V10 | 7.05 | On day 4, pH | 15 mL 50X UMG | Repurposed to |
| | | shift to 6.85 | | represent V01 |
| | | and temperature | | condition |
| | | shift to 38 | | |

UMG 100x amounts described above in Table 2.

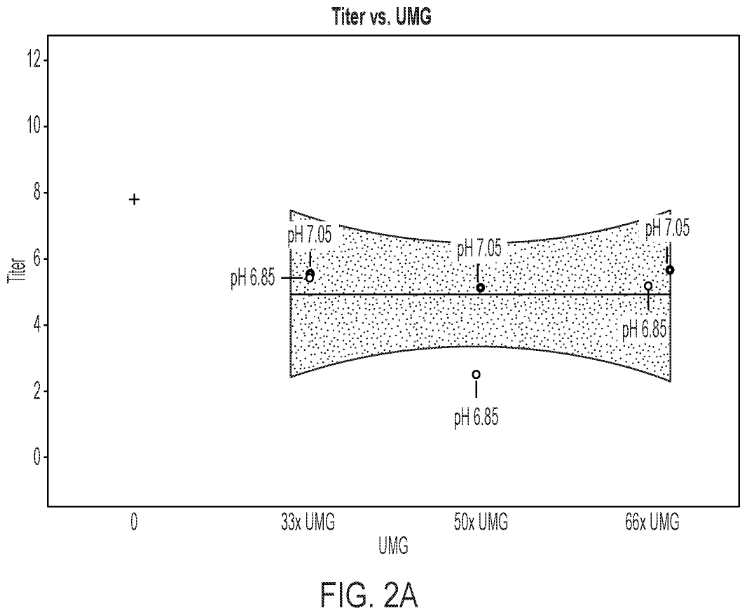

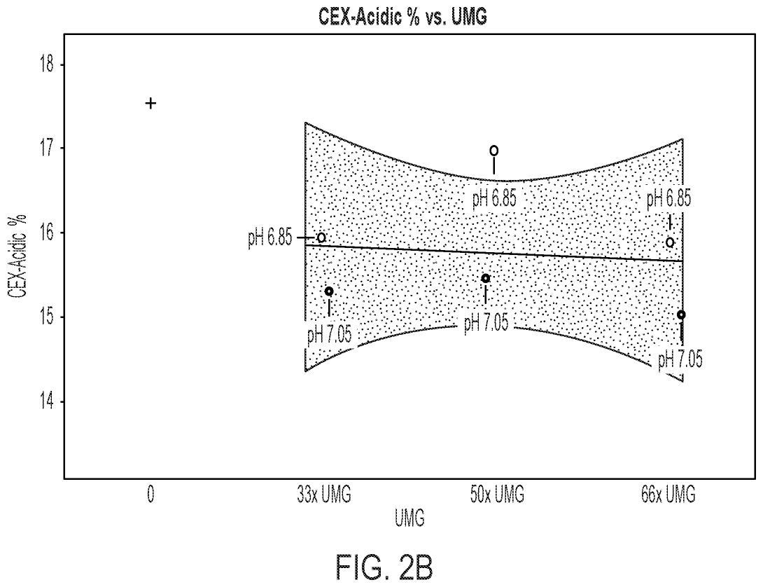

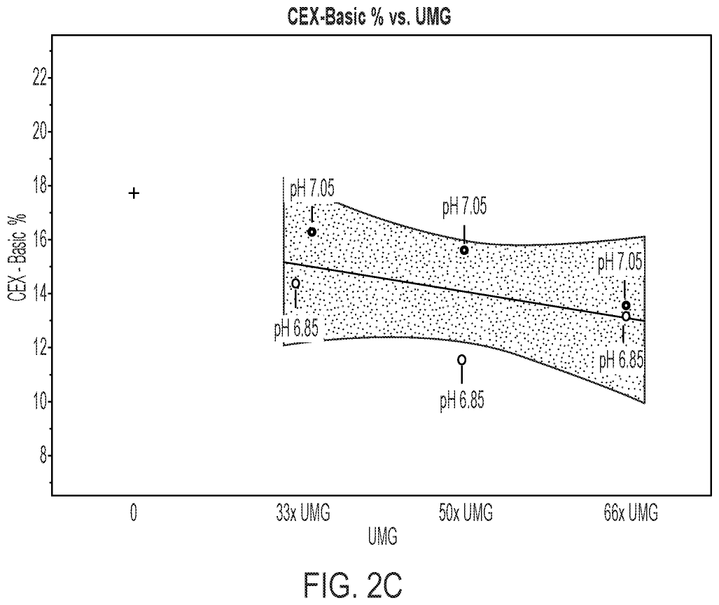

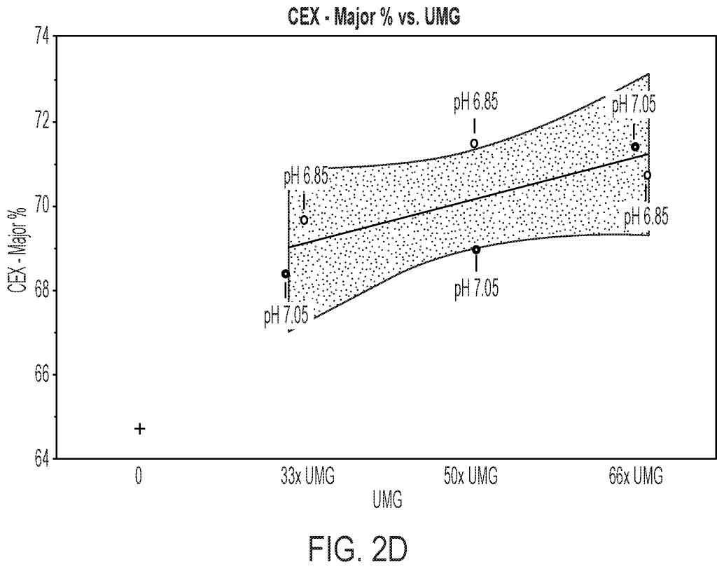

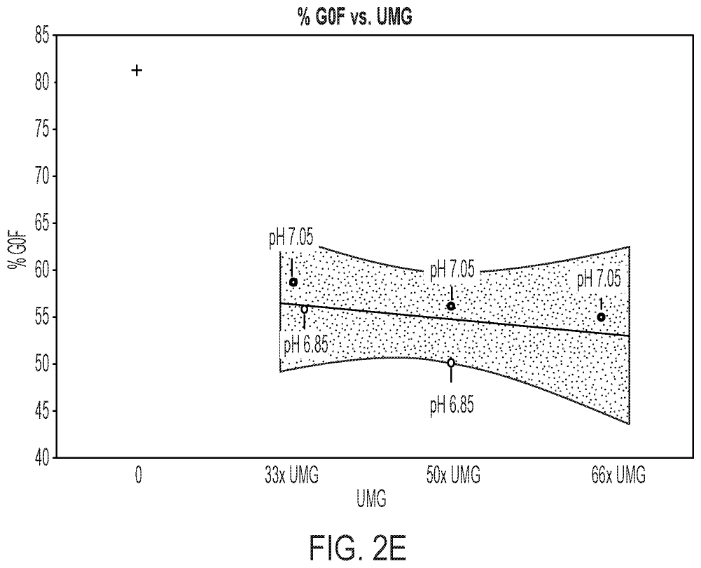

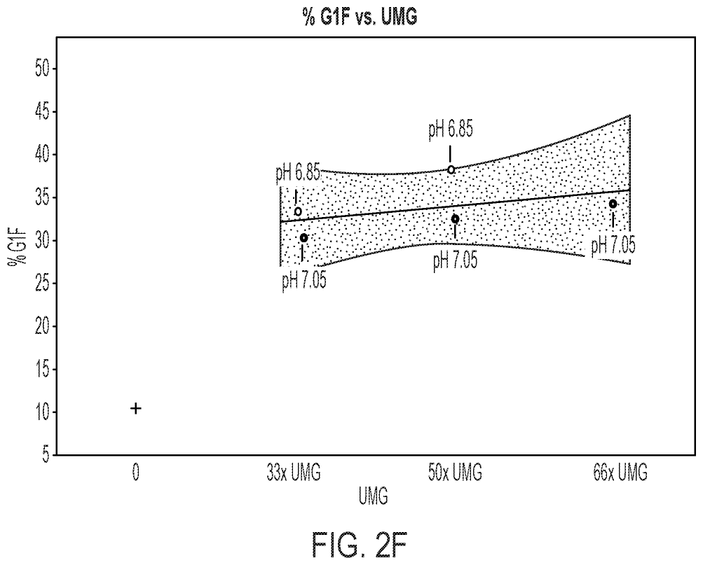

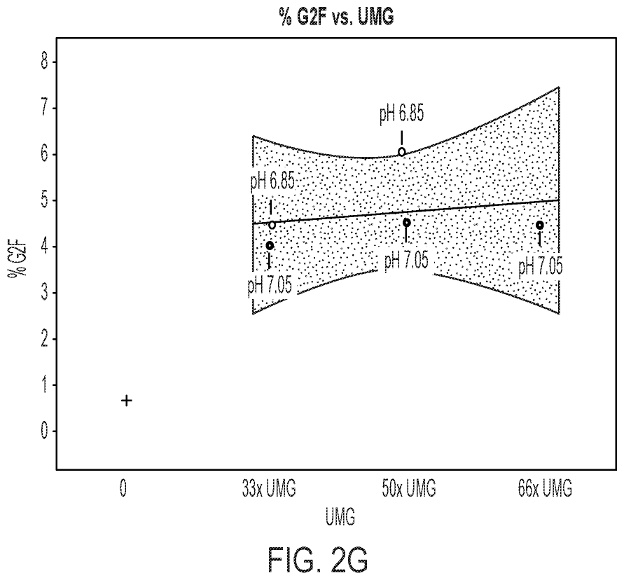

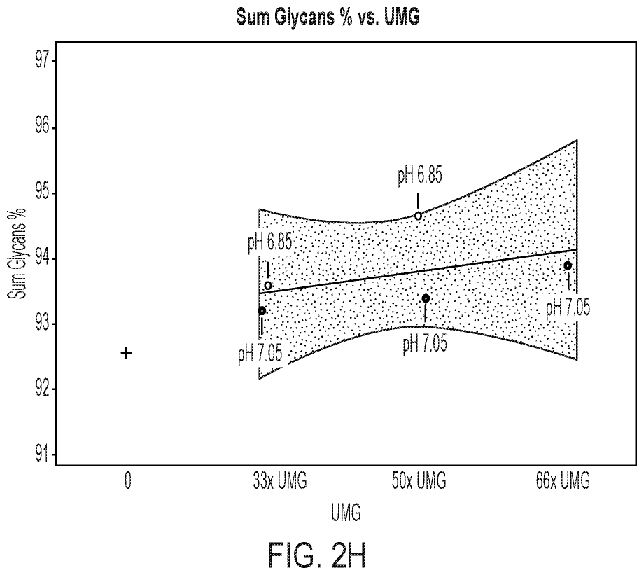

Results: Using the results from the experiments, prediction profiles were generated to further study the impact of the various conditions on cell culture and the product quality attributes of vedolizumab. The prediction profile results are described in FIGS. 2A-2H (antibody titer vs. UMG (FIG. 2A), acidic species % (CEX) vs. UMG (FIG. 2B), basic species % (CEX) vs. UMG (FIG. 2C), percentage of major species (CEX) vs. UMG (FIG. 2D), percentage of G0F species vs. UMG (FIG. 2E), percentage of G1F species (FIG. 2F), percentage of G2F species vs. UMG (FIG. 2G), and glycan sum vs. UMG (FIG. 2H)). In each of FIGS. 2A to 2H, vessels without UMG supplementation are shown by the dot to the far left of the shaded area (shaded area represents UMG supplementation). Further, the two pH amounts (pH 7.05 and pH 6.85) tested are shown in FIGS. 2A-2H.

As described in FIGS. 2D, 2F, 2G, and 2G, cell cultures with increasing UMG supplementation displayed a higher percentage of major species, G1F species, G2F species, and sum of glycans, respectively, relative to cultures without UMG supplementation. Further, cultures with UMG supplementation exhibited lower titer (FIG. 2A), lower acidic species (FIG. 2B), lower basic species (FIG. 2C), and lower G0F species (FIG. 2E) relative to cultures without UMG supplementation.

Across the different UMG concentrations tested, the concentration of UMG appeared to have minimal impact on titer and acidic species, a low impact on the percentage of basic species (i.e., basic species decreased slightly at higher UMG concentrations), and a higher impact on the percentage of major species (i.e., major species increased with higher UMG concentrations). There was no major change in carbohydrate profiles in response to the various UMG concentrations.

Finally, in regard to pH, operating at a low pH (6.85) appeared to perform better than operating at higher pH (pH 7.05), as described in FIGS. 2A-2H.

In a separate experiment, GS-CHO cells recombinantly expressing vedolizumab were cultured at 3000L scale in CD-CHO production medium, supplemented with a feed medium comprising uridine (20.91 mM mM concentration), manganese (0.039 mM concentration), galactose (96.69 mM concentration), and zinc (0.117 mM concentration). Feed was added daily to the culture beginning on day 4. Amounts of UMG added in the daily addition were as follows: 0.17 to 0.63 mM uridine, 0.31 to 1.2 μMganese, and 0.77 to 2.9 mM galactose. By the day of harvest, the average cumulative supplemental concentration of uridine in the production media after supplementing daily during days 4 to 13 of the culture was about 2.76 mM; the average cumulative supplemental concentration of the manganese was about 0.00515 mM; and the average cumulative supplemental concentration of the galactose was about 12.8 mM. Zinc was also added daily from days 4 to 13 as a feed supplement at a concentration of about 0.117 mM, where the average cumulative supplemental concentration in the production media by day 14 was about 0.0154 mM. Average used in this example is in reference to the average of the lots tested.

Antibody was harvested following 14 days in culture, and the level of fucosylated glycans was determined using HILIC following purification. Results are presented in Table 4.

TABLE 4
Representative Glycan Levels Following Cell Culture Supplementation
with Uridine, Manganese, Galactose, and Zinc

| Lot # | G0F + G1F + G2F | G1F | G0F | G2F |
|---|---|---|---|---|
| 1 | 93.08 | 35.47 | 52.01 | 5.60 |
| 2 | 93.05 | 35.59 | 51.89 | 5.57 |
| 3 | 92.94 | 36.23 | 50.57 | 6.14 |
| 4 | 93.17 | 35.29 | 52.35 | 5.53 |
| 5 | 92.74 | 35.43 | 51.70 | 5.61 |
| 6 | 92.86 | 36.26 | 50.45 | 6.15 |

### Example 3

### Effect of Lysine and Arginine on product quality attributes

The objective of this experiment was to test the effect of lysine and arginine levels in the feed medium on vedolizumab quality attributes, specifically titer and the percentage of basic species.

Design: The experiment was designed to assess the effects of lysine and arginine concentration on antibody titer when produced in GS-CHO cells, as well as the level of basic species (C-terminal lysine levels), as determined by CEX. Testing was performed in a manner similar to Examples 1 and 2.

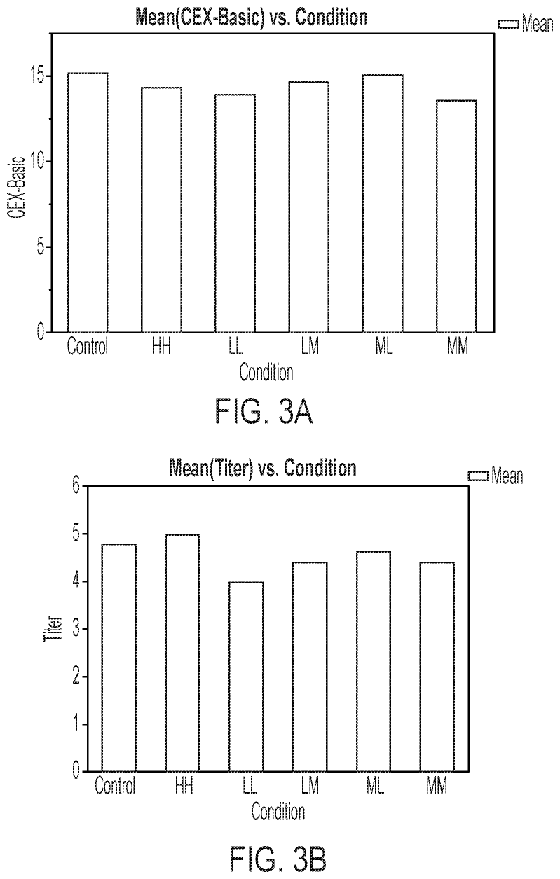

Results: Results comparing the effects of different arginine and lysine concentrations on the percentage of basic species are shown in FIG. 3A, and results showing impact on antibody titer are described in FIG. 3B. The labels on the X-axis of both FIGS. 3A and 3B correspond to high (H), medium (M), or low (L) concentrations of lysine and arginine, as outlined in Table 5. For example “LM” refers to a low level of lysine (see Table 5) and a medium level of arginine (see Table 5). These results indicate that low levels of lysine and arginine led to a minimal reduction in basic species, but that low levels of these amino acids negatively impacted antibody titer (5-4g, -20%) relative to the control.

TABLE 5
Summary of arginine and lysine concentrations

| Amino | Control | Low | Medium | High |
|---|---|---|---|---|
| Lysine (g/L) | 8.80 | 5.0 | 6.0 | 7.1 |
| Arginine (g/L) | 12.0 | 3.0 | 6.4 | 9.9 |

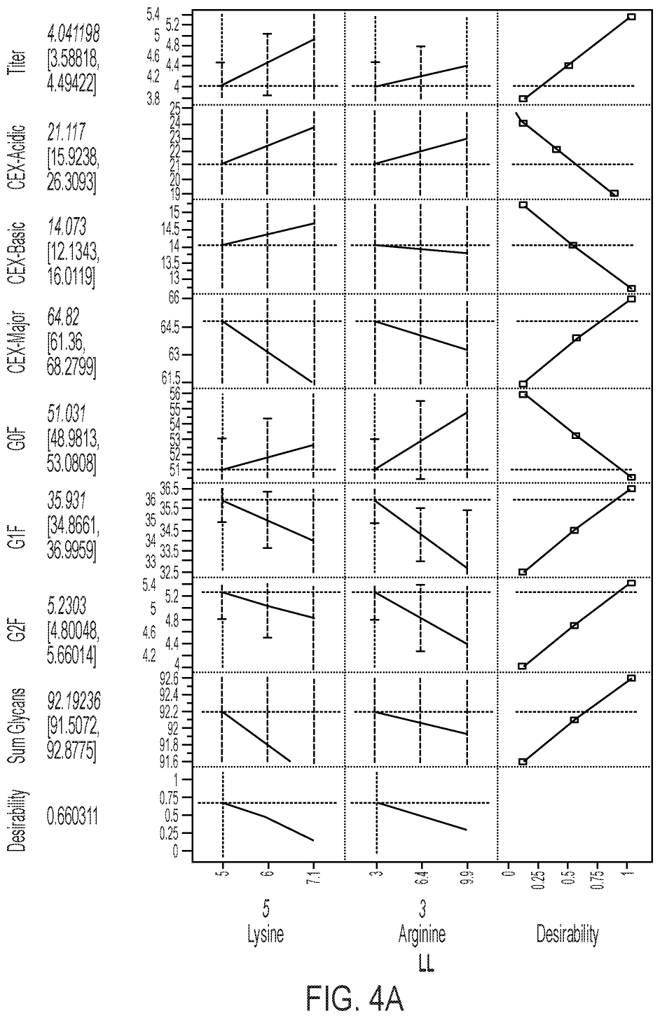

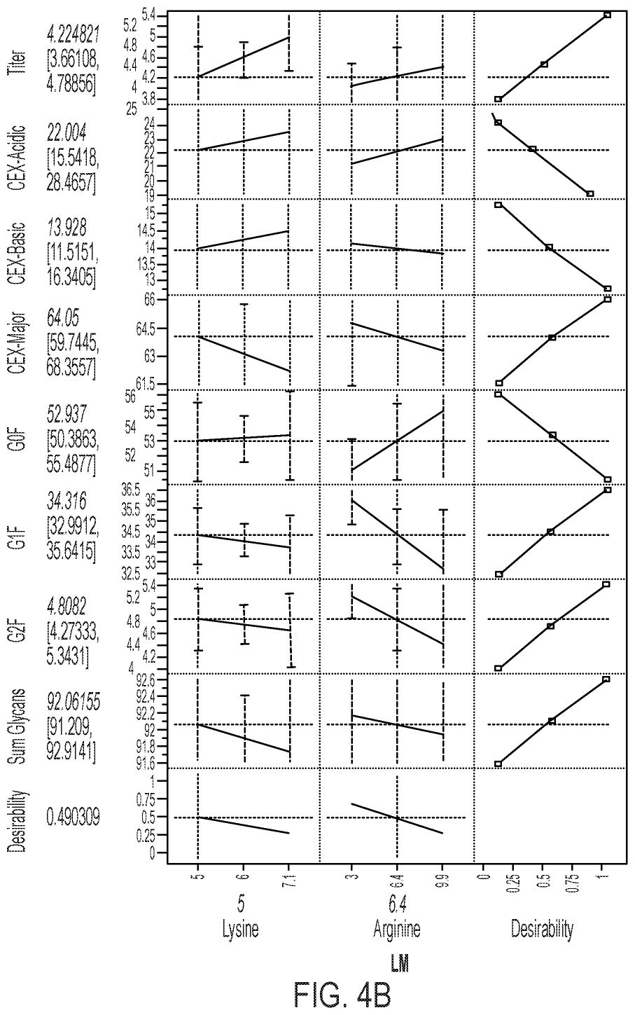

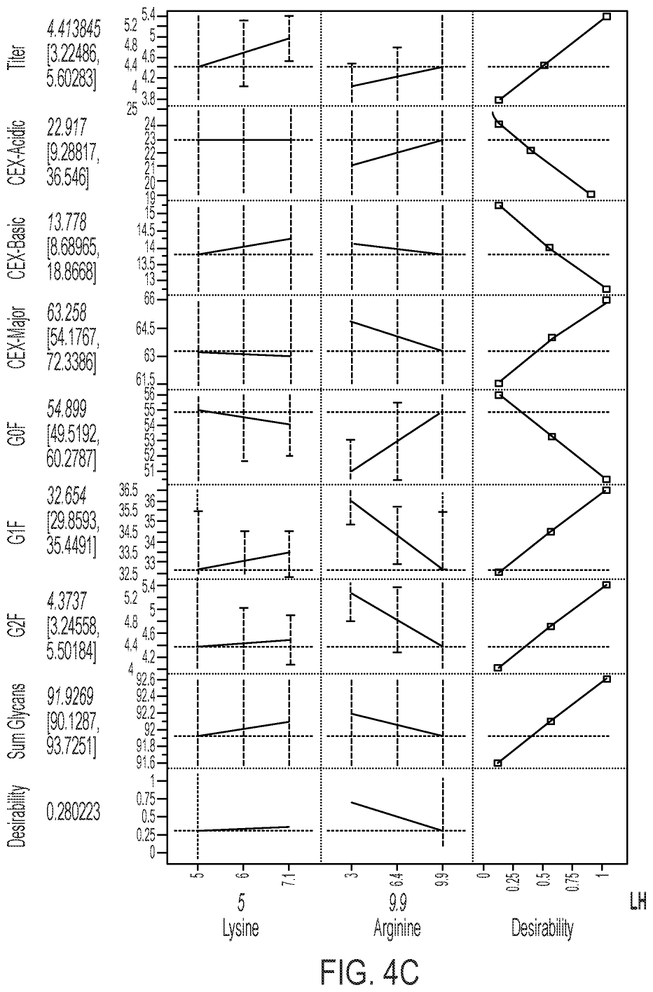

Predictive analysis was performed based on the arg/lys experiments. JMP analysis predicted that the optimal condition for reducing basic species is at low lysine and low arginine levels, (LL) as described in FIG. 4A. FIG. 4B and 4C show prediction profiles for “LM” (low lysine and medium arginine) “LH” (low lysine and high arginine) combinations. Because of the titer production impact, however, low lysine (5 g/L) and medium arginine (6.5 g/L) levels were used in Example 4.

### Example 4

### Effect of Zinc on Product Quality Attributes

The objective of this experiment was to test effect of zinc levels on vedolizumab quality attributes during cell culture, more specifically during the production phase of the culturing system.

Three levels of zinc were tested, as described below in Table 6. Culture ranged from harvest day 14 to 18. The zinc supplement was evaluated in combination of 22x UMG with reduced lysine and arginine (“LM” as described in Example 3). The concentrations of lysine and arginine in the feed were 5g/L and 6.4 g/L, respectively.

TABLE 6
Zinc concentration in feed medium during production phase

| | 0 Zinc* | 2 Zinc | 4 Zinc |
|---|---|---|---|
| [Zinc] uM | 14.3 | 28.6 | 57.2 |

*0 Zn means no additional Zn supplementation added to feed medium

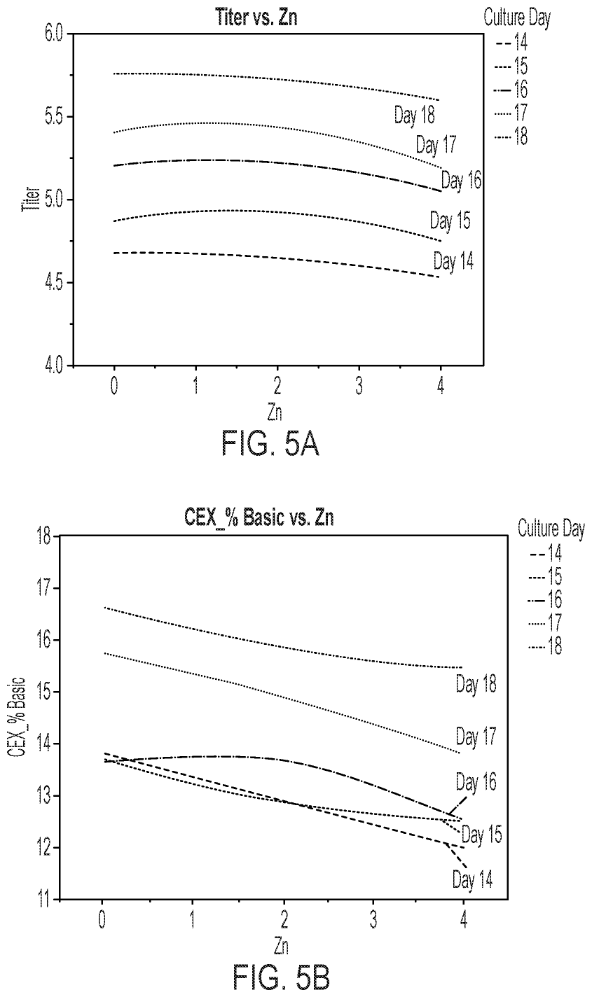

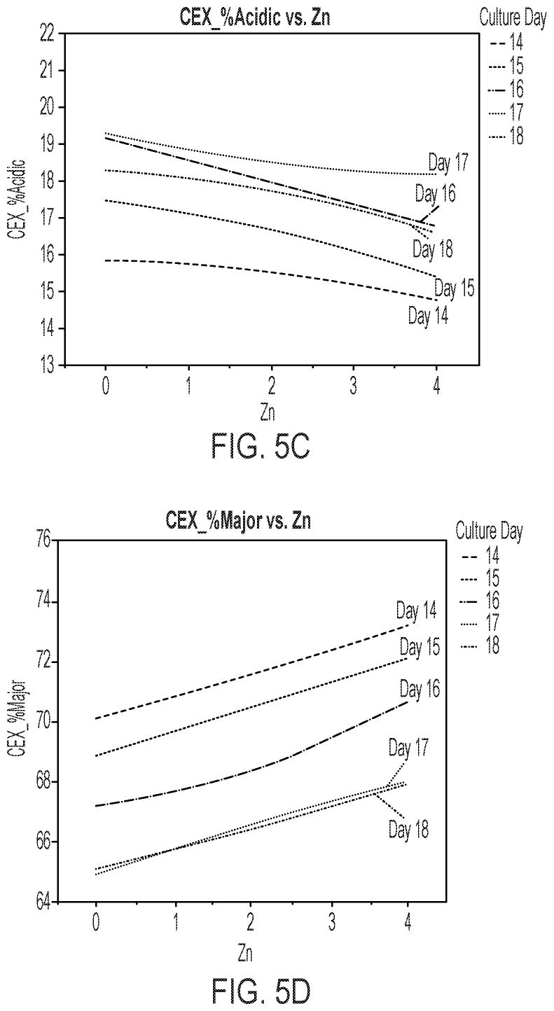

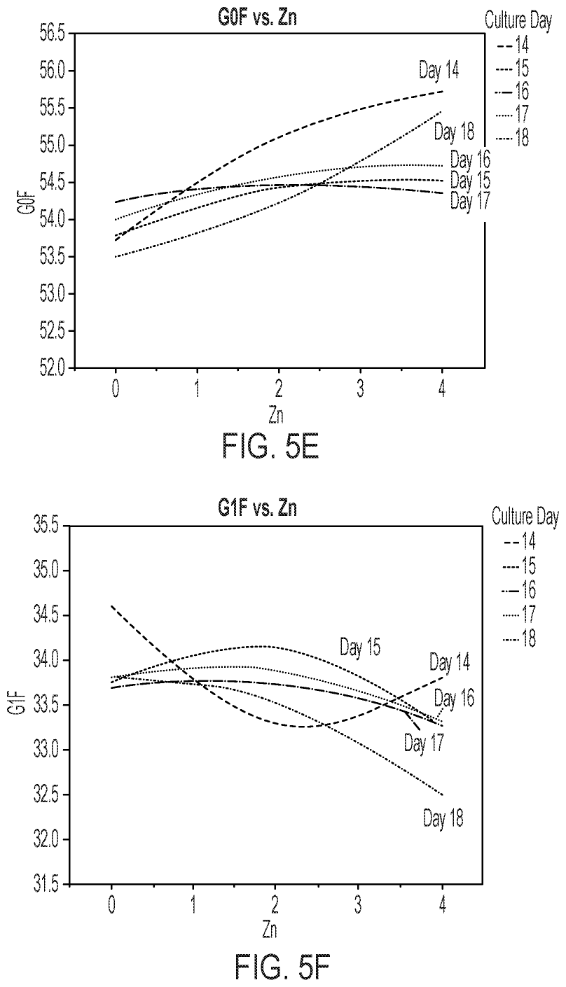

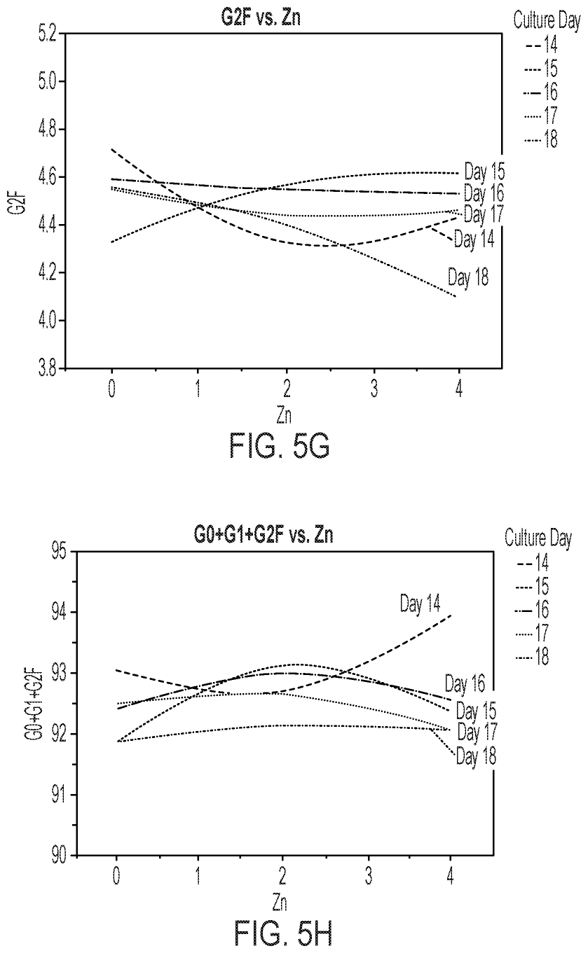

The tested zinc concentrations had no substantive impact on antibody titer, as described in FIG. 5A. Also described in Figure A is the impact on the number of production culture days, where prolonged culture days showed benefit on titer production. FIGS. 5B to 5G provide examining the impact of zinc vs. percentage of basic species (FIG. 5B), percentage of acidic species (FIG. 5C), percentage of major species (FIG. 5D), percentage of G0F species (FIG. 5E), percentage of G1F species (FIG. 5F), percentage of G2F species (FIG. 5G), and glycan species summation (FIG. 5H) after 14, 15, 16, 17, and 18 culture days.

Decreasing trends were observed in basic and acidic profiles with increasing levels of zinc, as described in FIGS. 5B and 5C. It was further observed that similar basic profiles were achieved up to culture day 16. The highest level of main (major) species was obtained at harvest day 14 with 57.2 uM zinc (4Zn), as described in FIG. 5D.

Zinc levels and culture day showed a minimal influence on carbohydrate profiles. Overall, the data in FIGS. 5A to 5H suggests ending culturing and harvesting no later than day 16.

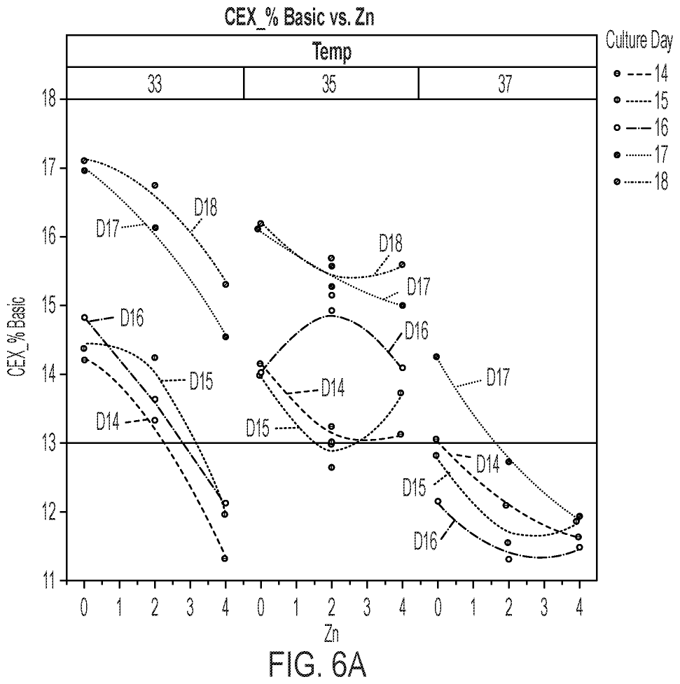

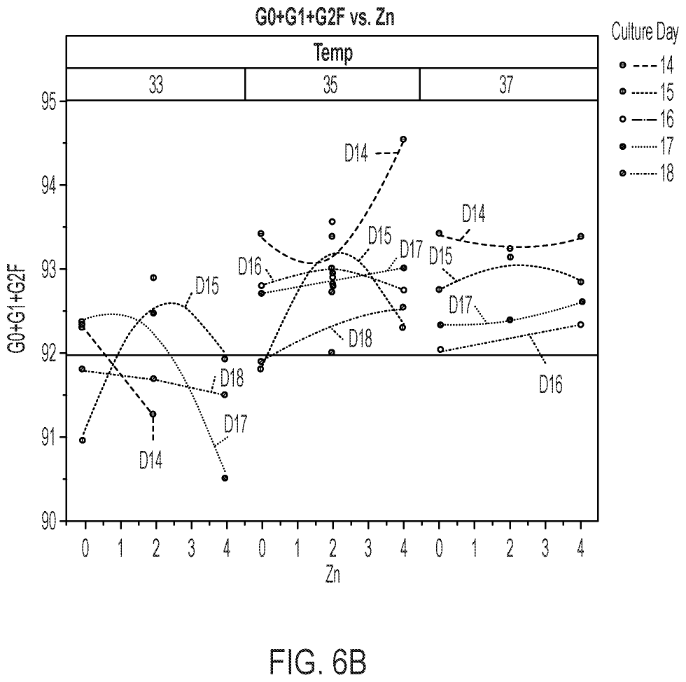

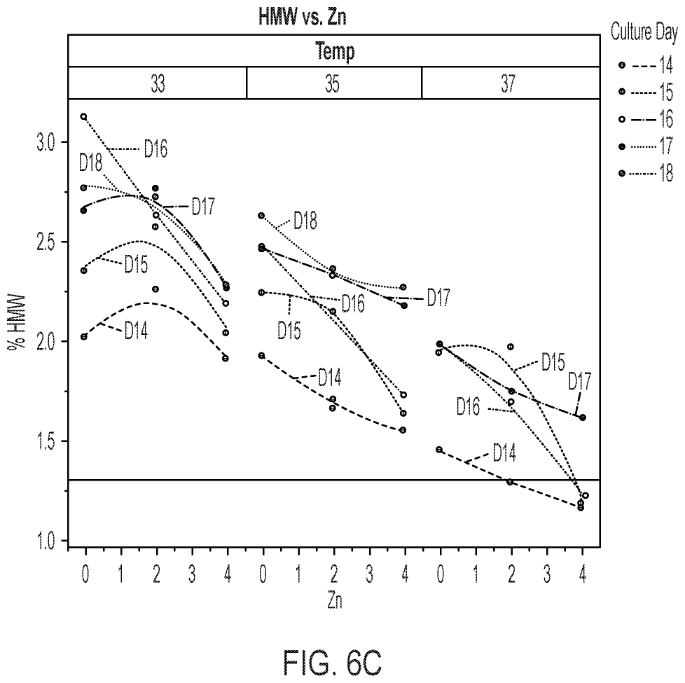

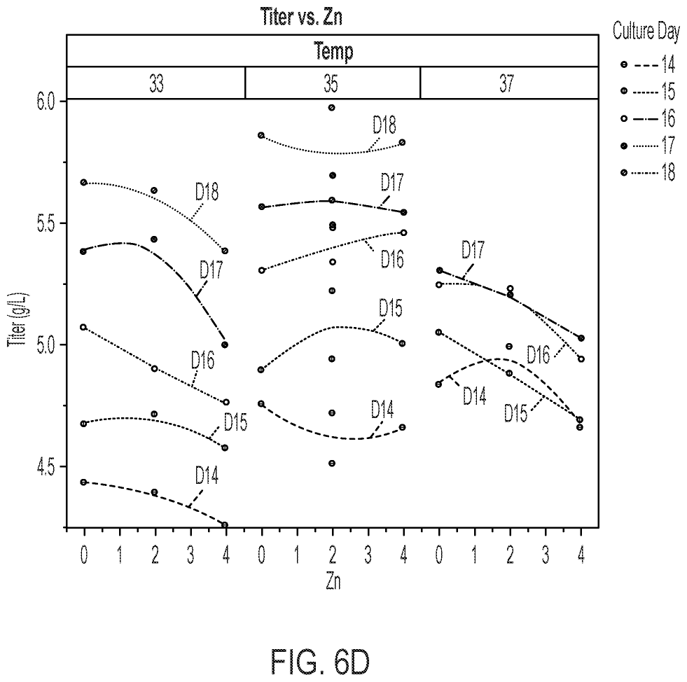

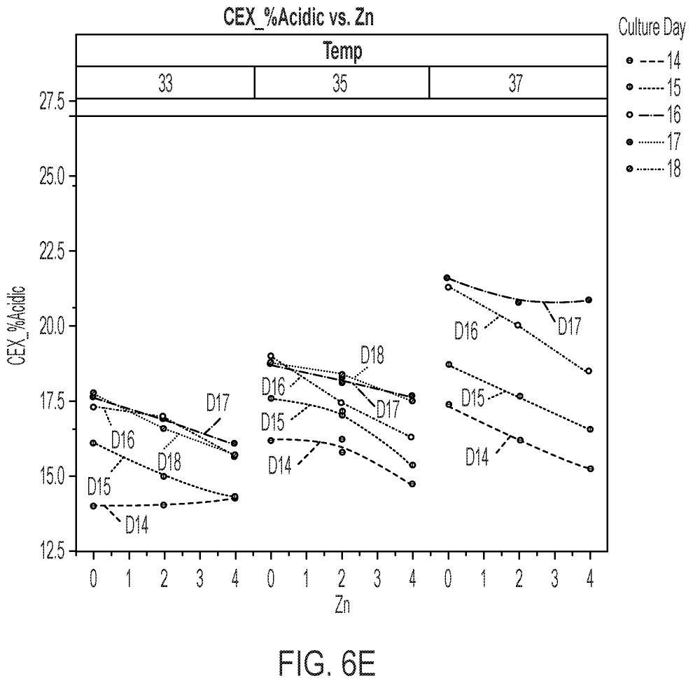

Temperature was also tested in combination with various zinc concentrations to determine whether there was an impact on various product qualities of vedolizumab. 33, 35, and 37 degree Celsius temperatures were tested in combination with 0 to 4 levels of zinc (see Table 6 above). Overall, 37 degrees was most effective at maintaining the desired vedolizumab product qualities. For example, FIG. 6A describes the % basic isoform of the antibody under various zinc conditions and temperatures. The level of percent basic species following supplementation with 57.2 uM zinc (4Zn) at 33° C. and 37° C. fell below the specified upper limit of basic species (isoform) (indicated by the black line, i.e., 13% basic antibody isoform (CEX)) and therefore met the specification requirement. In contrast, as shown in FIG. 6B, the glycan summation following supplementation with 57.2 uM zinc (4Zn) at 37° C. but not 33° C. fell above the lower acceptance criteria (indicated by black line). Further, it was observed that there was an increase in protein aggregation (HMW species; acceptance criteria indicated as about 1.4% or less) at lower temperatures, as described in FIG. 6C. The data in FIG. 6D suggests that titer production was benefited from prolonging culture days, and that 37° C. in generally generated more vedolizumab titer in comparison to 33° C. and 35° C. on day 14. The data in FIG. 6E suggests that acidic species of the antibody increased as the temperature increased and as culture elongation. The solid black line in FIG. 6E represents the upper acceptance limit of acidic species.

Overall, the data in FIGS. 6A to 6E suggested that it is best to stop fermentation by day 16 for production of vedolizumab in GS-CHO cells, and maintain an approximate 37 degree production temperature. Further 4 Zn provides a benefit as a supplement.

### Example 5

### Effect of Days in Culture on Product Quality Atributes

objective of this experiment was to test the effect of days in culture on vedolizumab quality attributes.

The percentage of acidic species of the antibody, percentage of basic species of the antibody, percentage of major species of the antibody, and titer of vedolizumab were assessed after 12, 13, 14, 15, 16, or 17 culture days in GS-CHO cells following two separate runs.

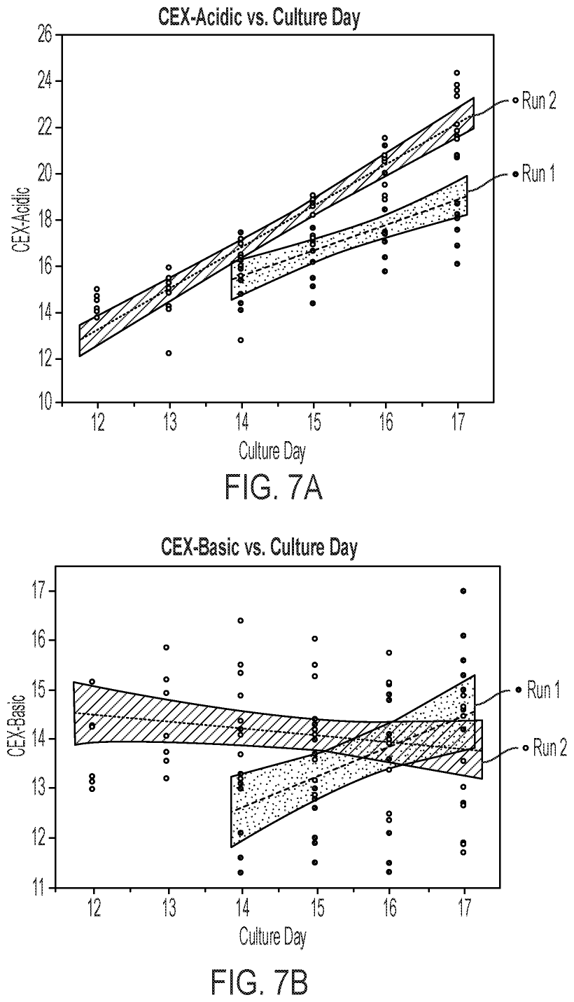

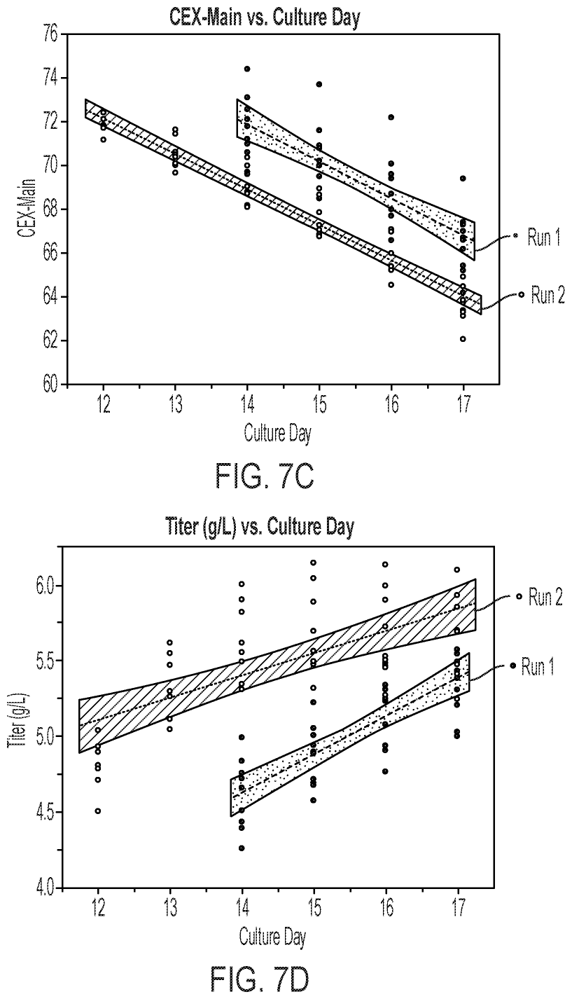

As shown in FIG. 7D, an increase in titer was observed as the number of culture days was extended. However, this titer increase was accompanied by an increase in acidic species and decrease in main species, as described in FIGS. 7A and 7B, respectively. The basic species did not appear to be impacted during Run 1 but a trend towards increasing basic species was observed during Run 2. The data indicate that it is generally best to stop fermentation and harvest by day 16. The lowest levels of acidic species were obtained when the antibody was harvested by day 15.

### Example 6

### Effect of pH on Product Quality Attributes

The objective of this experiment was to test the effect of pH during production phase cell culture on vedolizumab quality attributes. Specifically, introduction of a pH shift in the production phase culture medium was evaluated for impact on vedolizumab quality attributes.

The initial pH of the production phase culture medium was evaluated across a range of pH 6.8 to pH 7.2. During the pH shift, the pH of the culture medium was lowered to a final pH, which was evaluated across a range of pH 6.6 to pH 7.0. pH shift initiation time was studied from 86 hours to 108 hours, with a pH shift completion time (i.e., time to reach final pH) from 88 hours to 144 hours. The intervening 2-36 hour interval accounts for the pH ramp time.

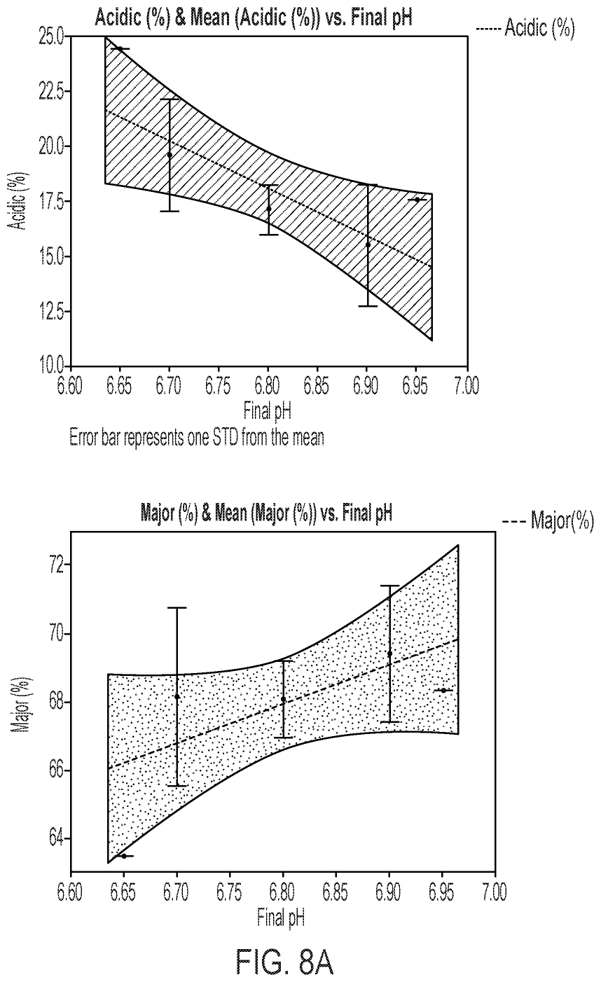

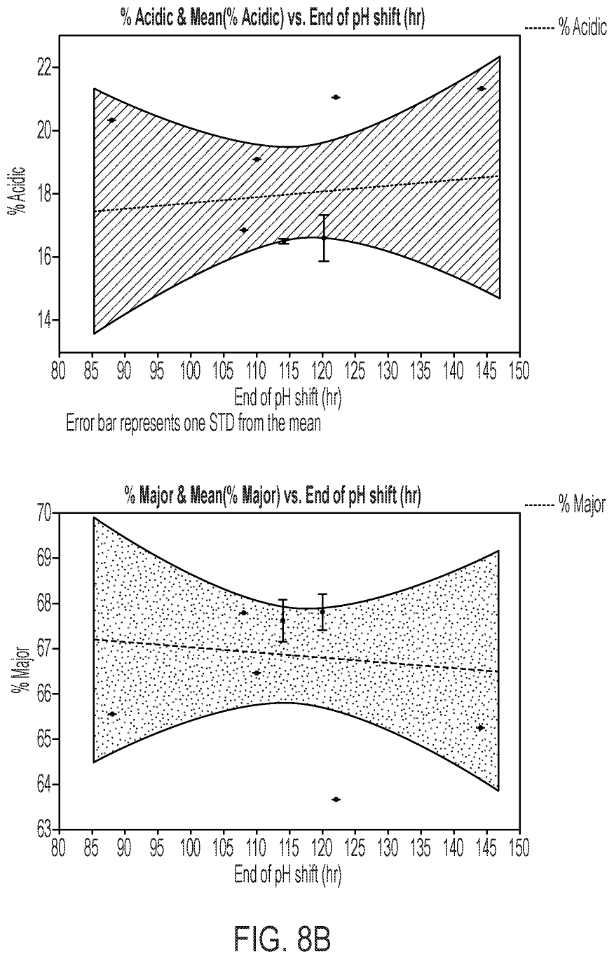

The highest % major antibody isoform (determined by CEX), and the lowest % acidic antibody isoform (determined by CEX) were observed at a final pH at or above pH 6.7 (FIG. 8A), and a pH shift completion time at or below 122 hours (FIG. 8B). This data suggests that a pH shift during production phase cell culture can reduce the level of acidic antibody isoform species in a vedolizumab preparation.

### Example 7

### Determination of Product Quality Attributes

The following analytical assays and methods were used in the foregoing examples to determine the product quality attributes of vedolizumab.

Cation exchange chromatography (CEX) fractionates vedolizumab antibody species (major isoform, basic species, and acidic species) according to overall surface charge. After dilution to low ionic strength using mobile phase, the test sample is injected onto a Dionex Pro-Pac™ WCX-10 column (Thermo Fisher Scientific, Waltham, Mass. (USA)) equilibrated in 10 mM sodium phosphate, pH 6.6, and eluted using a sodium chloride gradient in the same buffer. Protein elution is monitored at 280 nm and peaks are assigned to acidic, basic, or major isoforms categories. The percent major isoform, the sum of percent acidic species, and the sum of percent basic species are reported. The major isoform retention time of the sample is compared with that of the reference standard to determine the conformance.

The carbohydrate profile of vedolizumab is generated by the fractionation of free fluorescently-labeled carbohydrates by hydrophilic interaction phase separation (HILIC). Intact glycans are released from protein samples by digestion with N-glycosidase F, then immediately labeled with InstantAB fluorescent tag (Agilent Technologies, Inc., Santa Clara, Calif. (USA)) using the GlykoPrep Rapid Glycoprotein Sample Preparation System from Prozyme (Hayward, CA (USA)). The labeled glycans are fractionated using an ACQUITY UPLC BEH Amide Column (Waters Corporation, Milford, Mass. (USA)) and an acetonitrile/ammonium formate gradient system. Detection is achieved by a fluorescence emission at 344 nm using an excitation wavelength of 278 nm. An assay control was performed by determining the appropriate resolution of commercially available InstantAB-labeled glucose homopolymer ladder (Agilent Technologies, Inc., Santa Clara, Calif. (USA)). The quantitation is based on the relative area percent of detected sugars. The percent peak area of the G0F (asialo-, agalactosylated biantennary glycan, core fucosylated); G1F (asialo-, monogalactosylated biantennary glycan, core fucosylated); and G2F (asialo-, digalactosylated biantennary glycan, core fucosylated) species are reported.

Size-exclusion chromatography (SEC) is used to determine the purity of vedolizumab. Reference standard and test samples (75 ⱼ..t.g) are analyzed using two G3000 SWx1 columns (Tosoh Bioscience, King of Prussia, Pa. (USA)) connected in tandem and an isocratic phosphate-sodium chloride buffer system, pH 6.8. The method provides separation of antibody monomer from high molecular weight (HMW) species as well as low molecular weight (LMW) degradation products. Elution of protein species is monitored at 280 nm. The main peak (monomer) and the total peak area are assessed to determine purity. The purity (%) of the sample (calculated as % monomer) and the % aggregate are reported.

### Equivalents

Those skilled in the art will recognize, or be able to ascertain using no more than routine experimentation, many equivalents to the specific embodiments of the invention described herein. Such equivalents are intended to be encompassed by the following claims. The contents of all references, patents and published patent applications cited throughout this application are incorporated herein by reference.

SEQUENCE TABLE

| SEQ ID | | |
|---|---|---|
| NO: | DESCRIPTION | SEQUENCE |
| 1 | Heavy chain (HC) | QVQLVQSGAEVKKPGASVKVSCKGSGYTFTSYWMH |
| | variable region (amino | WVRQAPGQRLEWIGEIDPSESNTNYNQKFKGRVTLT |
| | acid) | VDISASTAYMELSSLRSEDTAVYYCARGGYDGWDY |
| | | AIDYWGQGTLVTVSS |
| 2 | HC CDR1 (amino | SYWMH |
| | acid) | |
| 3 | HC CDR2 (amino | EIDPSESNTNYNQKFKG |
| | acid) | |
| 4 | HC CDR3 (amino | GGYDGWDYAIDY |
| | acid) | |
| 5 | Light chain (LC) | DVVMTQSPLSLPVTPGEPASISCRSSQSLAKSYGNTYL |
| | variable region (amino | SWYLQKPGQSPQLLIYGISNRFSGVPDRFSGSGSGTDF |
| | acid) | TLKISRVEAEDVGVYYCLQGTHQPYTFGQGTKVEIK |
| 6 | LC CDR1 (amino | RSSQSLAKSYGNTYLS |
| | acid) | |
| 7 | LC CDR2 (amino | GISNRFS |
| | acid) | |
| 8 | LC CDR3 (amino | LQGTHQPYT |
| | acid) | |
| 9 | Light chain variable | GATGTAGTGATGACTCAAAGTCCACTCTCCCTGCCT |
| | region (nucleic acid) | GTCACCCCTGGAGAACCAGCTTCTATCTCTTGCAG |
| | | GTCTAGTCAGAGTCTTGCAAAGAGTTATGGGAACA |
| | | CCTATTTGTCTTGGTACCTGCAGAAGCCTGGCCAGT |
| | | CTCCACAGCTCCTCATCTATGGGATTTCCAACAGAT |
| | | TTTCTGGGGTGCCAGACAGGTTCAGTGGCAGTGGT |
| | | TCAGGGACAGATTTCACACTCAAGATCTCGCGAGT |
| | | AGAGGCTGAGGACGTGGGAGTGTATTACTGCTTAC |
| | | AAGGTACACATCAGCCGTACACGTTCGGACAGGGG |
| | | ACCAAGGTGGAGATCAAG |
| 10 | Heavy chain variable | CAGGTGCAATTGGTGCAGTCTGGGGCTGAGGTTAA |
| | region (nucleic acid) | GAAGCCTGGGGCTTCAGTGAAGGTGTCCTGCAAGG |
| | | GTTCTGGCTACACCTTCACCAGCTACTGGATGCATT |
| | | GGGTGAGGCAGGCGCCTGGCCAACGTCTAGAGTGG |
| | | ATCGGAGAGATTGATCCTTCTGAGAGTAATACTAA |
| | | CTACAATCAAAAATTCAAGGGACGCGTCACATTGA |
| | | CTGTAGACATTTCCGCTAGCACAGCCTACATGGAG |
| | | CTCTCCAGCCTGAGATCTGAGGACACTGCGGTCTA |
| | | CTATTGTGCAAGAGGGGGTTACGACGGATGGGACT |
| | | ATGCTATTGACTACTGGGGTCAAGGCACCCTGGTC |
| | | ACCGTCAGCTCA |
| 11 | Light chain (nucleic | GATGTAGTGATGACTCAAAGTCCACTCTCCCTGCCT |
| | acid) | GTCACCCCTGGAGAACCAGCTTCTATCTCTTGCAG |
| | | GTCTAGTCAGAGTCTTGCAAAGAGTTATGGGAACA |
| | | CCTATTTGTCTTGGTACCTGCAGAAGCCTGGCCAGT |
| | | CTCCACAGCTCCTCATCTATGGGATTTCCAACAGAT |
| | | TTTCTGGGGTGCCAGACAGGTTCAGTGGCAGTGGT |
| | | TCAGGGACAGATTTCACACTCAAGATCTCGCGAGT |
| | | AGAGGCTGAGGACGTGGGAGTGTATTACTGCTTAC |
| | | AAGGTACACATCAGCCGTACACGTTCGGACAGGGG |
| | | ACCAAGGTGGAGATCAAGCGTACGGTGGCTGCACC |
| | | ATCTGTCTTCATCTTCCCGCCATCTGATGAGCAGTT |
| | | GAAATCTGGAACTGCCTCTGTTGTGTGCCTGCTGA |
| | | ATAACTTCTATCCCAGAGAGGCCAAAGTACAGTGG |
| | | AAGGTGGATAACGCCCTCCAATCGGGTAACTCCCA |
| | | GGAGAGTGTCACAGAGCAGGACAGCAAGGACAGC |
| | | ACCTACAGCCTCAGCAGCACCCTGACCCTGAGCAA |
| | | AGCAGACTACGAGAAACACAAAGTCTACGCCTGCG |
| | | AAGTCACCCATCAGGGCCTGAGCTCGCCCGTCACA |
| | | AAGAGCTTCAACAGGGGAGAGTGT |
| 12 | Heavy chain (nucleic | CAGGTGCAATTGGTGCAGTCTGGGGCTGAGGTTAA |
| | acid) | GAAGCCTGGGGCTTCAGTGAAGGTGTCCTGCAAGG |
| | | GTTCTGGCTACACCTTCACCAGCTACTGGATGCATT |
| | | GGGTGAGGCAGGCGCCTGGCCAACGTCTAGAGTGG |
| | | ATCGGAGAGATTGATCCTTCTGAGAGTAATACTAA |
| | | CTACAATCAAAAATTCAAGGGACGCGTCACATTGA |
| | | CTGTAGACATTTCCGCTAGCACAGCCTACATGGAG |
| | | CTCTCCAGCCTGAGATCTGAGGACACTGCGGTCTA |
| | | CTATTGTGCAAGAGGGGGTTACGACGGATGGGACT |
| | | ATGCTATTGACTACTGGGGTCAAGGCACCCTGGTC |
| | | ACCGTCAGCTCAGCCTCCACCAAGGGCCCATCGGT |
| | | CTTCCCCCTGGCACCCTCCTCCAAGAGCACCTCTGG |
| | | GGGCACAGCGGCCCTGGGCTGCCTGGTCAAGGACT |
| | | ACTTCCCCGAACCGGTGACGGTGTCGTGGAACTCA |
| | | GGCGCCCTGACCAGCGGCGTGCACACCTTCCCGGC |
| | | TGTCCTACAGTCCTCAGGACTCTACTCCCTCAGCAG |
| | | CGTGGTGACCGTGCCCTCCAGCAGCTTGGGCACCC |
| | | AGACCTACATCTGCAACGTGAATCACAAGCCCAGC |
| | | AACACCAAGGTGGACAAGAAAGTTGAGCCCAAAT |
| | | CTTGTGACAAAACTCACACATGCCCACCGTGCCCA |
| | | GCACCTGAACTCGCGGGGGCACCGTCAGTCTTCCT |
| | | CTTCCCCCCAAAACCCAAGGACACCCTCATGATCT |
| | | CCCGGACCCCTGAGGTCACATGCGTGGTGGTGGAC |
| | | GTGAGCCACGAAGACCCTGAGGTCAAGTTCAACTG |
| | | GTACGTGGACGGCGTGGAGGTGCATAATGCCAAGA |
| | | CAAAGCCGCGGGAGGAGCAGTACAACAGCACGTA |
| | | CCGTGTGGTCAGCGTCCTCACCGTCCTGCACCAGG |
| | | ACTGGCTGAATGGCAAGGAGTACAAGTGCAAGGTC |
| | | TCCAACAAAGCCCTCCCAGCCCCCATCGAGAAAAC |
| | | CATCTCCAAAGCCAAAGGGCAGCCCCGAGAACCAC |
| | | AGGTGTACACCCTGCCCCCATCCCGGGATGAGCTG |
| | | ACCAAGAACCAGGTCAGCCTGACCTGCCTGGTCAA |
| | | AGGCTTCTATCCCAGCGACATCGCCGTGGAGTGGG |
| | | AGAGCAATGGGCAGCCGGAGAACAACTACAAGAC |
| | | CACGCCTCCCGTGCTGGACTCCGACGGCTCCTTCTT |
| | | CCTCTACAGCAAGCTCACCGTGGACAAGAGCAGGT |
| | | GGCAGCAGGGGAACGTCTTCTCATGCTCCGTGATG |
| | | CATGAGGCTCTGCACAACCACTACACGCAGAAGAG |
| | | CCTCTCCCTGTCTCCGGGTAAA |
| 13 | Heavy chain amino | QVQLVQSGAEVKKPGASVKVSCKGSGYTFTSYWMH |
| | acid sequence | WVRQAPGQRLEWIGEIDPSESNTNYNQKFKGRVTLT |
| | | VDISASTAYMELSSLRSEDTAVYYCARGGYDGWDY |
| | | AIDYWGQGTLVTVSSASTKGPSVFPLAPSSKSTSGGT |
| | | AALGCLVKDYFPEPVTVSWNSGALTSGVHTFPAVLQ |
| | | SSGLYSLSSVVTVPSSSLGTQTYICNVNHKPSNTKVD |
| | | KKVEPKSCDKTHTCPPCPAPELAGAPSVFLFPPKPKD |
| | | TLMISRTPEVTCVVVDVSHEDPEVKFNWYVDGVEVH |
| | | NAKTKPREEQYNSTYRVVSVLTVLHQDWLNGKEYK |
| | | CKVSNKALPAPIEKTISKAKGQPREPQVYTLPPSRDEL |
| | | TKNQVSLTCLVKGFYPSDIAVEWESNGQPENNYKTT |
| | | PPVLDSDGSFFLYSKLTVDKSRWQQGNVFSCSVMHE |
| | | ALHNHYTQKSLSLSPGK |
| 14 | Light chain amino | DVVMTQSPLSLPVTPGEPASISCRSSQSLAKSYGNTYL |
| | acid sequence | SWYLQKPGQSPQLLIYGISNRFSGVPDRFSGSGSGTDF |
| | | TLKISRVEAEDVGVYYCLQGTHQPYTFGQGTKVEIKR |
| | | TVAAPSVFIFPPSDEQLKSGTASVVCLLNNFYPREAK |
| | | VQWKVDNALQSGNSQESVTEQDSKDSTYSLSSTLTL |
| | | SKADYEKHKVYACEVTHQGLSSPVTKSFNRGEC |

1. A method of producing a composition comprising a humanized anti-α4137 antibody, said method comprising
culturing a mammalian host cell in a production medium, and
adding a supplement comprising uridine, manganese, and galactose to the production medium, thereby producing a composition comprising the humanized anti-α4β7 antibody,
wherein the mammalian host cell is genetically engineered to express a humanized anti-α4β7 antibody which is an IgG1 antibody, comprises a heavy chain variable region comprising a CDR3 domain as set forth in SEQ ID NO: 4, a CDR2 domain as set forth in SEQ ID NO: 3, and a CDR1 domain as set forth in SEQ ID NO: 2; and comprises a light chain variable region comprising a CDR3 domain as set forth in SEQ ID NO: 8, a CDR2 domain as set forth in SEQ ID NO: 7, and a CDR1 domain as set forth in SEQ ID NO: 6.

2. The method of claim 1, wherein:
the composition comprises a decreased amount of basic isoform (as determined by Cation Exchange Chromatography (CEX)) of the humanized anti-α4β7 antibody in comparison to a control mammalian host cell expressing the humanized anti-α4β7 antibody that is cultured under substantially similar conditions but in the absence of the supplement; or
the composition comprises about 16% or less, about 14% or less, about 13% or less basic isoform (as determined by CEX) of the humanized anti-α4β7 antibody.

3-6. (canceled)

7. The method of claim 1, wherein the cumulative concentration of uridine added in the production medium between supplementation and harvest is about 1 to about 7 mM; wherein the cumulative concentration of manganese in the production medium between supplementation and harvest is about 0.002 to about 0.015 mM; and/or wherein the cumulative concentration of galactose in the production medium between supplementation and harvest is about 3 to about 20 mM.

8-15. (canceled)

16. The method of claim 2, wherein the feed medium further comprises zinc.

17. (canceled)

18. The method of claim 2, wherein the method further decreases the percentage of acidic species of the humanized anti-α4β7 antibody relative to the percentage of acidic species produced in a control mammalian host cell expressing the humanized anti-α4β7 antibody that is cultured under substantially similar conditions but in the absence of the supplement.

19-20. (canceled)

21. A method of producing a composition comprising a humanized anti-α4β7 antibody, said method comprising culturing a mammalian host cell in a production medium comprising zinc, thereby producing a composition comprising the humanized anti-α4β7 antibody,
wherein the mammalian host cell is genetically engineered to express a humanized anti-α4β7 antibody which is an IgG1 antibody, comprises a heavy chain variable region comprising a CDR3 domain as set forth in SEQ ID NO: 4, a CDR2 domain as set forth in SEQ ID NO: 3, and a CDR1 domain as set forth in SEQ ID NO: 2; and comprises a light chain variable region comprising a CDR3 domain as set forth in SEQ ID NO: 8, a CDR2 domain as set forth in SEQ ID NO: 7, and a CDR1 domain as set forth in SEQ ID NO: 6.

22. The method of claim 21, wherein:
the composition comprises a decreased amount of basic isoform of the humanized anti-α4β7 antibody in comparison to a control mammalian host cell expressing the humanized anti-α4β7 antibody that is cultured under the same conditions in the absence of zinc or
the composition comprises about 16% or less, about 14% or less, about 13% or less basic isoform of the humanized anti-α4β7 antibody.

23-25. (canceled)

26. The method of claim 21 any one of claims 21, wherein the concentration of zinc in the production medium is 2 μM to 60 μM.

27-28. (canceled)

29. The method of claim 1, wherein the production medium comprises 5.0 to 8.8 g/L lysine and 3.0 to 12.0 g/L arginine.

30-35. (canceled)

36. A method of producing a composition comprising a humanized anti-α4β7 antibody, said method comprising
culturing a mammalian host cell in a growth medium in an expansion phase, wherein the mammalian host cell is genetically engineered to express a humanized anti-α4β7 antibody, and
culturing the mammalian host cell in a production medium in a production phase, such that a composition comprising the humanized anti-α4β7 antibody is produced,
wherein the mammalian host cell is cultured at a temperature that is approximately the same in both the expansion phase and the production phase, and
wherein the humanized anti-α4β7 antibody is an IgG1 antibody; comprises a heavy chain variable region comprising a CDR3 domain as set forth in SEQ ID NO: 4, a CDR2 domain as set forth in SEQ ID NO: 3, and a CDR1 domain as set forth in SEQ ID NO: 2; and comprises a light chain variable region comprising a CDR3 domain as set forth in SEQ ID NO: 8, a CDR2 domain as set forth in SEQ ID NO: 7, and a CDR1 domain as set forth in SEQ ID NO: 6.

37. The method of claim 36, wherein the method is a method of producing a composition comprising high levels of monomer (as determined by SEC) of the humanized anti-α4β7 antibody, relative to a control culture cultured under substantially similar conditions but with a different temperature between the expansion and the production phases.

38. The method of claim 36, wherein:
the temperature is from 36 to 38 degrees Celsius:,
the average temperature is from 36.5 to 37.5 degrees Celsius; or
the temperature is an average temperature of about 37 degrees Celsius.

39-45. (canceled)

46. The method of claim 1, wherein the production medium has a glucose level that is maintained at about 7 g/L or less a during the production phase.

47-55. (canceled)

56. The method of claim 1, wherein the humanized anti-α4β7 antibody comprises a heavy chain variable domain comprising an amino acid sequence as set forth in SEQ ID NO: 1, and comprises a light chain variable domain comprising an amino acid sequence as set forth in SEQ ID NO: 5.

57. The method of claim 1, wherein the humanized anti-α4β7 antibody is vedolizumab.

58. The method of claim 1, wherein the method comprises harvesting and purification of the antibody.

59. (canceled)

60. The method of claim 58, wherein the method further comprises preparing a pharmaceutical formulation of the purified antibody which is suitable for human therapeutic use.

61-64. (canceled)

65. A composition comprising humanized anti-α4β7 antibodies wherein the composition is produced using or is obtainable by the method of claim 1.

66. (canceled)

67. The composition of claim 65, comprising a population of humanized anti-α4β7 antibodies having (i) 90% or more, or (ii) 92-95%, total asialo-, agalacto, core fucosylated biantennary glycan (G0F), asialo-, monogalacto, core fucosylated biantennary glycan (G1F), and/or asialo-, digalacto, core fucosylated biantennary glycan (G2F) glycosylation variants.

68. The method of claim 1, wherein the method is a method of producing a composition having a decreased amount of a G0F glycoform (as determined by Hydrophilic Interaction Chromatography (HILIC) of the humanized anti-α4β7 antibody, in comparison to a control mammalian host cell expressing the humanized anti-α4β7 antibody that is cultured under substantially similar conditions but in the absence of the supplement.

69-70. (canceled)

71. The method of claim 1, wherein the method is a method of producing a composition having about 65% or less, about 60% or less, or about 55% or less G0F glycoform (as determined by HILIC) of the humanized anti-α4β7 antibody.

72-84. (canceled)

85. The method of claim 68, wherein the method further increases the percentage of main isoform species of the humanized anti-α4β7 antibody relative to the percentage of main isoform species produced in a control mammalian host cell expressing the humanized anti-α4β7 antibody that is cultured under substantially similar conditions in the absence of a feed medium comprising uridine, manganese, and galactose added to the production medium.

86-88. (canceled)

89. The method of claim 21, wherein the method comprises harvesting and purification of the antibody.

90. (canceled)

91. The method of claim 89, wherein the method further comprises preparing a pharmaceutical formulation of the purified antibody which is suitable for human therapeutic use.

92-95. (canceled)

96. A composition comprising a humanized anti-α4β7 antibody, wherein the composition is obtainable by the method of claim 21.

97. A cell culture comprising a host cell genetically engineered to express a humanized anti-α4β7 antibody, and a production medium supplemented with uridine, manganese, and galactose (UMG), wherein the humanized anti-α4β7 antibody is an IgG1 antibody and comprises a heavy chain variable region comprising the amino acid sequence set forth in SEQ ID NO:1, and a light chain variable region comprising the amino acid sequence set forth in SEQ ID NO: 5.

98. The cell culture of claim 97, wherein the production medium comprises supplemented uridine at a concentration of about 1 to about 7 mM, supplemented manganese at a concentration of about 0.002 to about 0.015 mM, and supplemented galactose at a concentration of about 3 to about 20 mM on the day of harvest.

99. The cell culture of claim 97, wherein the production medium further comprises zinc.

100-101. (canceled)

102. A cell culture comprising a host cell genetically engineered to express a humanized anti-α4β7 antibody, and a production medium supplemented with zinc, wherein the humanized anti-α4β7 antibody is an IgG1 antibody and comprises a heavy chain variable region comprising the amino acid sequence set forth in SEQ ID NO:1, and a light chain variable region comprising the amino acid sequence set forth in SEQ ID NO: 5.

103. The cell culture of claim 102, wherein the production medium comprises supplemented zinc at a concentration of about 0.005 mM to 0.045 mM on the day of harvest.

104. The cell culture of claim 102, wherein the production medium further comprises uridine, manganese, and galactose (UMG).

105-106. (canceled)

107. The cell culture of claim 97, wherein the expressed humanized anti-α4β7 antibody has an isoform distribution comprising:
a. 16% or less, 15% or less, 14% or less, 13% or less, or 12% or less basic isoform; and/or
b. at least 65%, at least 68%, at least 70%, at least 72%, or at least 75% major isoform.

108. (canceled)

109. The cell culture of claim 97, wherein the expressed humanized anti-α4β7 antibody has a total fucosylated N-glycan content (G0F+G1F+G2F) of at least 88%, at least 90%, at least 91%, at least 92%, at least 93%, at least 94%, or at least 95%.

110-111. (canceled)

112. The cell culture of claim 97, wherein the cell culture further comprises arginine and/or lysine.

113-114. (canceled)

115. A humanized anti-α4β7 antibody produced by the cell culture of claim 97.

116. A method of producing a composition comprising a humanized anti-α4β7 antibody, said method comprising
culturing a mammalian host cell genetically engineered to express the humanized anti-α4β7 antibody in a first production medium having a first pH; and
culturing the mammalian host cell in a second production medium having a second pH; wherein the second pH is lower than the first pH, and
wherein the humanized anti-α4β7 antibody is an IgG1 antibody; comprises a heavy chain variable region comprising a CDR3 domain as set forth in SEQ ID NO: 4, a CDR2 domain as set forth in SEQ ID NO: 3, and a CDR1 domain as set forth in SEQ ID NO: 2; and comprises a light chain variable region comprising a CDR3 domain as set forth in SEQ ID NO: 8, a CDR2 domain as set forth in SEQ ID NO: 7, and a CDR1 domain as set forth in SEQ ID NO: 6.

117. (canceled)

118. The method of claim 116, wherein the first pH is in the range of pH 6.8-7.2, and wherein the second pH is in the range of pH 6.7-6.95.

119-121. (canceled)

122. The method of claim 116, further comprising harvesting the anti-α4β7 antibody from the second production medium.

123. (canceled)

124. The method of claim 116, wherein the composition has an increased level of the anti-α4β7 antibody major isoform, relative to a control composition in which the mammalian host cell is cultured at the first pH without a pH shift.

125. A composition comprising a humanized anti-α4β7 antibody, wherein the composition is produced using or is obtainable by the method of claim 36.

126. A composition comprising a humanized anti-α4β7 antibody, wherein the composition is produced using or is obtainable by the method of claim 116.


SEQUENCE LISTING (14 sequences)

SEQ ID NO: 1 - protein, 121 aa, Artificial Sequence [sequence omitted]

SEQ ID NO: 2 - protein, 5 aa, Artificial Sequence
  Description of Artificial Sequence Synthetic peptide
SYWMH

SEQ ID NO: 3 - protein, 17 aa, Artificial Sequence
  Description of Artificial Sequence Synthetic peptide
EIDPSESNTNYNQKFKG

SEQ ID NO: 4 - protein, 12 aa, Artificial Sequence
  Description of Artificial Sequence Synthetic peptide
GGYDGWDYAIDY

SEQ ID NO: 5 - protein, 112 aa, Artificial Sequence [sequence omitted]

SEQ ID NO: 6 - protein, 16 aa, Artificial Sequence
  Description of Artificial Sequence Synthetic peptide
RSSQSLAKSYGNTYLS

SEQ ID NO: 7 - protein, 7 aa, Artificial Sequence
  Description of Artificial Sequence Synthetic peptide
GISNRFS

SEQ ID NO: 8 - protein, 9 aa, Artificial Sequence
  Description of Artificial Sequence Synthetic peptide
LQGTHQPYT

SEQ ID NO: 9 - DNA, 336 bp, Artificial Sequence
  Description of Artificial Sequence Synthetic polynucleotide
gatgtagtgatgactcaaagtccactctccctgcctgtcacccctggagaaccagcttctatctcttgcaggtctagtcagagtcttgcaaagagttatgggaacacctatttgtcttggtacctgcagaagcctggccagtctccacagctcctcatctatgggatttccaacagattttctggggtgccagacaggttcagtggcagtggttcagggacagatttcacactcaagatctcgcgagtagaggctgaggacgtgggagtgtattactgcttacaaggtacacatcagccgtacacgttcggacaggggaccaaggtggagatcaag

SEQ ID NO: 10 - DNA, 363 bp, Artificial Sequence
  Description of Artificial Sequence Synthetic polynucleotide
caggtgcaattggtgcagtctggggctgaggttaagaagcctggggcttcagtgaaggtgtcctgcaagggttctggctacaccttcaccagctactggatgcattgggtgaggcaggcgcctggccaacgtctagagtggatcggagagattgatccttctgagagtaatactaactacaatcaaaaattcaagggacgcgtcacattgactgtagacatttccgctagcacagcctacatggagctctccagcctgagatctgaggacactgcggtctactattgtgcaagagggggttacgacggatgggactatgctattgactactggggtcaaggcaccctggtcaccgtcagctca

SEQ ID NO: 11 - DNA, 657 bp, Artificial Sequence
  Description of Artificial Sequence Synthetic polynucleotide
gatgtagtgatgactcaaagtccactctccctgcctgtcacccctggagaaccagcttctatctcttgcaggtctagtcagagtcttgcaaagagttatgggaacacctatttgtcttggtacctgcagaagcctggccagtctccacagctcctcatctatgggatttccaacagattttctggggtgccagacaggttcagtggcagtggttcagggacagatttcacactcaagatctcgcgagtagaggctgaggacgtgggagtgtattactgcttacaaggtacacatcagccgtacacgttcggacaggggaccaaggtggagatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgaccctgagcaaagcagactacgagaaacacaaagtctacgcctgcgaagtcacccatcagggcctgagctcgcccgtcacaaagagcttcaacaggggagagtgt

SEQ ID NO: 12 - DNA, 1353 bp, Artificial Sequence
  Description of Artificial Sequence Synthetic polynucleotide
caggtgcaattggtgcagtctggggctgaggttaagaagcctggggcttcagtgaaggtgtcctgcaagggttctggctacaccttcaccagctactggatgcattgggtgaggcaggcgcctggccaacgtctagagtggatcggagagattgatccttctgagagtaatactaactacaatcaaaaattcaagggacgcgtcacattgactgtagacatttccgctagcacagcctacatggagctctccagcctgagatctgaggacactgcggtctactattgtgcaagagggggttacgacggatgggactatgctattgactactggggtcaaggcaccctggtcaccgtcagctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcgcgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 13 - protein, 451 aa, Artificial Sequence
  Description of Artificial Sequence Synthetic polypeptide
QVQLVQSGAEVKKPGASVKVSCKGSGYTFTSYWMHWVRQAPGQRLEWIGEIDPSESNTNYNQKFKGRVTLTVDISASTAYMELSSLRSEDTAVYYCARGGYDGWDYAIDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 219 aa, Artificial Sequence
  Description of Artificial Sequence Synthetic polypeptide
DVVMTQSPLSLPVTPGEPASISCRSSQSLAKSYGNTYLSWYLQKPGQSPQLLIYGISNRFSGVPDRFSGSGSGTDFTLKISRVEAEDVGVYYCLQGTHQPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC